(19) United States
(12) Patent Application Publication
(10) Pub. No.: US 2024/0374709 A1
(43) Pub. Date: Nov. 14, 2024

(54) VACCINE COMPOSITIONS

(71) Applicant: EMERGENT PRODUCT DEVELOPMENT GAITHERSBURG INC., Gaithersburg, MD (US)

(72) Inventors: James Paul LATA, Silver Spring, MD (US); Kelly Lyn WARFIELD, North Potomac, MD (US); Michael Joseph LACY, Damascus, MD (US); Jee Loon LOOK, Boyds, MD (US); Christian Fernando RUIZ, Derwood, MD (US)

(21) Appl. No.: 18/690,650

(22) PCT Filed: Sep. 15, 2022
(86) PCT No.: PCT/US2022/076494

§ 371 Date: Mar. 8, 2024

Related U.S. Application Data
(60) Provisional application No. 63/244,931, filed on Sep. 16, 2021.

(51) Int. Cl. A61K 39/145; A61K 39/00; A61K 39/39; A61K 47/02; A61K 47/18; A61K 47/26; A61P 37/04

(52) U.S. Cl. CPC A61K 39/145; A61K 39/39; A61K 47/02; A61K 47/18; A61K 47/26; A61P 37/04; A61K 2039/55505; A61K 2039/55561; A61K 2039/70

(57) ABSTRACT

The present invention relates to a vaccine composition comprising an influenza Type A hemagglutinin stabilized stem nanoparticle (HA-ss-np); an aluminum hydroxide; a synthetic oligodeoxynucleotide adjuvant containing at least one CpG motif (CpG ODN); and a phosphate salt, wherein the HA-ss-np is not substantially adsorbed to the aluminum hydroxide, and wherein at least a portion of the CpG ODN is adsorbed to the aluminum hydroxide in the composition. The present disclosure also provides a method of inducing an immunological response against an influenza virus in a subject in need thereof, comprising administering an immunologically effective amount of the vaccine composition described herein. The present disclosure further provides a method of inducing an immunological response against an influenza virus in a subject in need thereof, comprising administering a dose of about 20 μg to about 300 μg of an HA-ss-np in a vaccine composition, wherein the vaccine composition further comprises an aluminum hydroxide; CpG ODN; and a phosphate salt, and wherein the HA-ss-np is not substantially adsorbed to the aluminum hydroxide, and wherein at least a portion of the CpG ODN is adsorbed to the aluminum hydroxide. Also provided herein is a method of producing a vaccine composition, comprising combining HA-ss-np with an adjuvant mixture, wherein the adjuvant mixture comprises a diluent solution comprising a phosphate salt; aluminum hydroxide; and CpG ODN, wherein the adjuvant mixture comprises CpG ODN-adsorbed aluminum hydroxide, and wherein the HA-ss-np is not substantially adsorbed to the aluminum hydroxide.

### SEQUENCE LISTING

The instant application contains a Sequence Listing which has been submitted electronically in XML format and is hereby incorporated by reference in its entirety. Said Sequence Listing XML, created on Sep. 14, 2022, is named 0195-0011WO1_ST26.xml and is 18,158 bytes in size.

### FIELD OF THE INVENTION

The present disclosure relates to vaccine compositions comprising an influenza Type A hemagglutinin stabilized stem nanoparticle (HA-ss-np); aluminum hydroxide; a synthetic oligodeoxynucleotide containing at least one CpG motif (CpG ODN); and a phosphate salt, wherein the HA-ss-np is not substantially adsorbed to the aluminum hydroxide, and wherein at least a portion of the CpG ODN is adsorbed to the aluminum hydroxide in the composition. The present disclosure also provides a method of inducing an immunological response against an influenza virus comprising administering a vaccine composition. Also provided is a method of producing a vaccine composition.

### BACKGROUND

Influenza viruses are one of the most ubiquitous viruses, affecting both humans and livestock. Influenza results in significant economic burden, morbidity and even mortality.

Influenza viruses cause epidemics almost every winter, with infection rates for type A or B virus as high as 40% over a six-week period. Influenza infection results in various disease states, from a sub-clinical infection through mild upper respiratory infection to a severe viral pneumonia. Typical influenza epidemics cause increases in incidence of pneumonia and lower respiratory disease as witnessed by increased rates of hospitalization or mortality. The severity of the disease is primarily determined by the age of the host, the host's immune status and the site of infection.

Vaccination plays a critical role in controlling annual influenza epidemics. However, the unpredictability of the dominant influenza strain(s) each season creates challenges in vaccine development, and inaccurate strain prediction greatly diminishes vaccine efficacy for an entire season.

### SUMMARY OF THE INVENTION

In embodiments, the disclosure provides a vaccine composition comprising an influenza Type A hemagglutinin stabilized stem nanoparticle (HA-ss-np); aluminum hydroxide; a synthetic oligodeoxynucleotide containing at least one CpG motif (CpG ODN); and a phosphate salt, wherein the HA-ss-np is not substantially adsorbed to the aluminum hydroxide, and wherein at least a portion of the CpG ODN is adsorbed to the aluminum hydroxide in the composition.

In embodiments, the disclosure provides a vaccine composition comprising: about 0.002% w/v to about 0.015% w/v of a first hemagglutinin stabilized stem nanoparticle (HA-ss-np) comprising an HA-ss from a Group 1 influenza A virus; about 0.002% w/v to about 0.015% w/v of a second hemagglutinin stabilized stem nanoparticle (HA-ss-np) comprising an HA-ss from a Group 2 influenza A virus; about 0.02% w/v to about 0.08% w/v of aluminum hydroxide; about 0.08% w/v to about 0.12% w/v of CPG 7909; about 0.35 mM to about 0.7 mM sodium phosphate monobasic monohydrate; about 0.010% w/v to about 0.02% w/v of a polysorbate; about 1 mM to about 10 mM Tris-HCl; about 50 mM to about 200 mM NaCl; and about 1% w/v to about 10% w/v sucrose, wherein the first HA-ss-np and the second HA-ss-np are not substantially adsorbed to the aluminum hydroxide, and wherein at least a portion of the CPG 7909 is adsorbed to the aluminum hydroxide.

In embodiments, the disclosure provides a method of inducing an immunological response against an influenza virus in a subject in need thereof, comprising administering an immunologically effective amount of the vaccine composition described herein.

In embodiments, the disclosure provides a method of inducing an immunological response against an influenza virus in a subject in need thereof, comprising administering a dose of about 20 μg to about 300 μg of an HA-ss-np in a vaccine composition, wherein the vaccine composition further comprises aluminum hydroxide; CpG ODN; and a phosphate salt, and wherein the HA-ss-np is not substantially adsorbed to the aluminum hydroxide, and wherein at least a portion of the CpG ODN is adsorbed to the aluminum hydroxide.

In embodiments, the disclosure provides a method of producing a vaccine composition, comprising: combining HA-ss-np with an adjuvant mixture, wherein the adjuvant mixture comprises a diluent solution comprising a phosphate salt; aluminum hydroxide; and CpG ODN, wherein the adjuvant mixture comprises CpG ODN-adsorbed aluminum hydroxide, and wherein the HA-ss-np is not substantially adsorbed to the aluminum hydroxide.

### BRIEF DESCRIPTION OF THE DRAWINGS

The following drawings form part of the present specification and are included to further demonstrate exemplary embodiments of certain aspects of the present invention.

FIG. 1 shows exemplary results of percent survival of mice vaccinated with formulations comprising an influenza Type A, subtype H1 hemagglutinin stabilized stem nanoparticle (H1-ss-np) and different adjuvant combinations and challenged with influenza A virus, as described in embodiments herein.

FIG. 2 shows exemplary results of the HA titer and anti-ferritin/anti-HA ratios of mice vaccinated with formulations comprising H1-ss-np and different adjuvant combinations and challenged with influenza A virus, as described in embodiments herein.

FIG. 3 shows exemplary results of percent survival, weight loss protection, and weight stability of mice vaccinated with formulations comprising H1-ss-np and different adjuvant combinations and challenged with influenza A virus, as described in embodiments herein.

FIG. 4 shows exemplary results of virus neutralization titer measured in mice vaccinated with formulations comprising H1-ss-np and different adjuvant combinations and challenged with influenza A virus, as described in embodiments herein.

FIG. 5 shows exemplary results of percent survival of mice vaccinated with formulations comprising H1-ss-np and different adjuvant combinations and challenged with influenza A virus, as described in embodiments herein.

FIG. 6 shows an exemplary graphical representation of the results in FIG. 5.

FIG. 7 shows exemplary results of percent survival of mice vaccinated with formulations comprising H1-ss-np and varying combinations and amounts of Alhydrogel+CPG 7909, and challenged with influenza A virus, as described in embodiments herein.

FIG. 8 shows exemplary results of weight loss protection of mice vaccinated with formulations comprising H1-ss-np and varying combinations and amounts of Alhydrogel+CPG 7909, and challenged with influenza A virus, as described in embodiments herein.

FIG. 9 shows exemplary results of immune response end point titer measured in mice vaccinated with formulations comprising H1-ss-np and varying combinations and amounts of Alhydrogel+CpG 7909, and challenged with influenza A virus, as described in embodiments herein.

FIG. 10 shows exemplary results of percent survival for mice vaccinated with formulations comprising H1-ss-np and different adjuvant combinations, and subsequently challenged with influenza A virus, as described in embodiments herein.

FIG. 11 shows exemplary results of weight loss protection of mice vaccinated with formulations comprising H1-ss-np and varying combinations and amounts of Alhydrogel+CPG 7909, and challenged with influenza A virus, as described in embodiments herein.

FIG. 12 shows exemplary results of immune response end point titer measured in mice vaccinated with formulations comprising H1-ss-np and varying combinations and amounts Alhydrogel+CPG 7909, and challenged with influenza A virus, as described in embodiments herein.

FIGS. 13A and 13B show exemplary results of survival and immune response end point titer measured in mice vaccinated with monovalent or bivalent formulations comprising H1-ss-np and/or H10-ss-np and Alhydrogel+CPG 7909, and challenged with influenza A virus, as described in embodiments herein. Survival is shown as a percentage. The formulations in FIG. 13A included dilutions of a proposed human dose of 20 μg each H1 and H10-ss-np+400 μg CPG 7909+600 μg aluminum as Alhydrogel. The formulations in FIG. 13B included dilutions of a proposed human dose of 20 μg each H1 and H10-ss-np+500 μg CPG 7909+300 μg aluminum as Alhydrogel.

FIG. 14 shows exemplary results of percent survival of mice vaccinated with formulations comprising H10-ss-np and varying combinations and amounts of Alhydrogel+CPG 7909, and challenged with influenza A virus, as described in embodiments herein.

FIG. 15 shows exemplary results of weight loss protection of mice vaccinated with formulations comprising H10-ss-np and varying combinations and amounts of Alhydrogel+CPG 7909, and challenged with influenza A virus, as described in embodiments herein.

FIGS. 16A and 16B show exemplary results of a freeze-thaw study to evaluate the effect of 5% sucrose on antigen aggregation after six freeze-thaw cycles as described in embodiments herein. FIG. 16A shows the percent intact nanoparticles and percent aggregation as measured by SEC-HPLC. FIG. 16B shows the percent intact nanoparticles and percent aggregation as measured by DLS.

FIG. 17 shows an exemplary particle size distribution of the formulation in Table 7 after 0 or 1 freeze-thaw cycle.

FIG. 18 shows exemplary vaccine formulations to assess antigen and CpG ODN binding to Alhydrogel (“Alum”), as described in embodiments herein.

FIGS. 19A and 19B show exemplary results of the percent free antigen and percent free CpG ODN at different sodium phosphate concentrations. FIG. 19A shows the binding results for the high Alum/low CpG ODN formulations shown in FIG. 18. FIG. 19B shows the binding results for the low Alum/high CpG ODN formulations shown in FIG. 18.

FIGS. 20A and 20B show exemplary results of the percent free antigen and percent free CpG ODN in 0.4 mM phosphate over a period of 6 days. FIG. 20A shows the stability results for the high Alum/low CpG ODN formulations shown in FIG. 18. FIG. 20B shows the stability results for the low Alum/high CpG ODN formulations shown in FIG. 18.

FIG. 21 shows exemplary results of the percent survival of mice vaccinated with formulations comprising H1-ss-np and varying combinations and amounts of Alhydrogel+CPG 7909, and challenged with influenza A virus, as described in embodiments herein.

FIG. 22 shows exemplary results of the percent survival of mice vaccinated with formulations comprising H1-ss-np and varying combinations and amounts of Alhydrogel+CPG 7909, and challenged with influenza A virus, as described in embodiments herein.

### DETAILED DESCRIPTION OF THE INVENTION

In embodiments, the present disclosure provides a vaccine composition comprising an influenza antigen; an aluminum salt; a synthetic oligodeoxynucleotide containing at least one CpG motif (CpG ODN); and a binding modulator, wherein the antigen is not substantially adsorbed to the aluminum salt, and wherein at least a portion of the CpG ODN is adsorbed to the aluminum salt in the composition.

Vaccine compositions disclosed herein effectively induce an immune response against influenza virus, without substantial antigen adsorption to the aluminum salt. In general, aluminum salts typically require adsorption of antigens onto the aluminum salt particles, which subsequently triggers an immune response to the antigens, e.g., production of antigen-specific antibodies. It was therefore surprisingly discovered that vaccine compositions disclosed herein, in which the antigen, e.g., HA-ss-np, is not substantially adsorbed to the aluminum salt, e.g., aluminum hydroxide, were highly effective in inducing an antigen-specific immune response.

In embodiments, a vaccine composition comprises an influenza Type A hemagglutinin stabilized stem nanoparticle (HA-ss-np); aluminum hydroxide; a synthetic oligodeoxynucleotide containing at least one CpG motif (CpG ODN); and a phosphate salt, wherein the HA-ss-np is not substantially adsorbed to the aluminum hydroxide, and wherein at least a portion of the CpG ODN is adsorbed to the aluminum hydroxide in the composition.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y and any numbers that fall within x and y. Reference to any numerical range expressly includes each numerical value (including fractional numbers and whole numbers) encompassed by that range. To illustrate, a range of “at least 10” or “at least about 10” includes whole numbers of 10, 11, 12, 13, 14, 15, 16, 17, 18, 19, 20, etc., and fractional numbers 10.1, 10.2 10.3, 10.4, 10.5, 10.6, 10.7, 10.8, 10.9, etc. In a further illustration, reference herein to a range of “less than 10” or “less than about 10” includes whole numbers 9, 8, 7, 6, 5, 4, 3, 2, 1, 0, etc., and fractional numbers 9.9, 9.8, 9.7, 9.6, 9.5, 9.4, 9.3, 9.2, 9.1, 9.0, etc.

As used herein, the term “substantially,” or “substantial,” when used in a negative connotation refers to the complete or near complete lack of an action, characteristic, property, state, structure, item, or result. For example, a composition that is “substantially” free of a certain component would not have any amount of that component, or the component would be present in such a low amount in the composition that the effect would be the same as if the component were not present.

As used herein, the term “in embodiments” refers to in some embodiments and does not necessarily apply to all embodiments.

In embodiments, the disclosure provides compositions that consist of or consist essentially of the recited components at the recited amounts. In compositions that consist essentially of the recited components, such compositions specifically exclude components that reduce the immunogenic effectiveness of the composition and/or components that materially change the recited adsorption characteristics of the aluminum hydroxide, i.e., wherein the HA-ss-np is not substantially adsorbed to the aluminum hydroxide and wherein at least a portion of the CpG ODN is adsorbed to the aluminum hydroxide.

In embodiments, the disclosure provides a vaccine composition comprising an antigen. In embodiments, the antigen comprises an influenza virus antigen. In some embodiments, the antigen is within or part of a nanoparticle. In embodiments, the antigen is a HA-ss-np. In embodiments, the antigen elicits an immune response against a broad range of influenza viruses.

As used herein, an immune response to a vaccine provided herein is the development in a subject of a humoral and/or cellular immune response to an antigen present in the vaccine. As used herein, a “humoral immune response” refers to an immune response mediated by antibody molecules, including IgM, IgA and IgG. A “cellular immune response” is mediated by T-lymphocytes and/or other white blood cells. One type of cellular immunity involves an antigen-specific response by cytolytic T-cells (“CTLs”). CTLs have specificity for peptide antigens that are presented in association with proteins encoded by major histocompatibility complex (MHC) and expressed on the surfaces of cells. CTLs help induce and promote the destruction of intracellular microbes, e.g., viruses, by causing lysis of cells infected with such microbes. Another aspect of cellular immunity involves an antigen-specific response by helper T-cells. Helper T-cells act to help stimulate the function and focus the activity of non-specific effector cells against cells displaying peptide antigens in association with MHC molecules on their surface. A cellular immune response also refers to the production of cytokines, chemokines, and other such molecules produced by activated T-cells and/or other white blood cells, including those derived from CD4+ and CD8+ T-cells. In embodiments, the immune response stimulates CTLs. In embodiments, the immune response stimulates the production and/or activation of helper T-cells, chemokines and/or cytokines. In embodiments, the immune response comprises an antibody-mediated immune response, e.g., stimulates the production of antibodies (e.g., IgA, IgM, and/or IgG) by B-cells. In embodiments, the immune response activates suppressor, cytotoxic, or helper T-cells and/or T-cells directed specifically to the antigen present in the vaccine composition provided herein. In embodiments, the immune response neutralizes or decreases infectivity of the virus and/or mediates antibody-complement or antibody-dependent cell cytotoxicity (ADCC) to provide protection to an immunized individual from the virus. Methods of assessing immune responses include immunoassays and neutralization assays.

In embodiments, the immune response comprises producing neutralizing antibodies. As used herein, neutralizing antibodies are antibodies that prevent an influenza virus from completing one round of replication. As defined herein, one round of replication refers to the life cycle of the virus, starting with attachment of the virus to a host cell and ending with budding of newly formed virus from the host cell. For influenza virus, this life cycle includes, but is not limited to, the steps of attaching to a cell, entering a cell, cleavage and rearrangement of the HA protein, fusion of the viral membrane with the endosomal membrane, release of viral ribonucleoproteins into the cytoplasm, formation of new viral particles and budding of viral particles from the host cell membrane. A neutralizing antibody may inhibit or prevent the virus from completing one or more of these steps, e.g., attachment.

As used throughout the specification, all nomenclature used to classify influenza virus is that commonly used by one of ordinary skill in the art. Thus, a Type of influenza virus refers to influenza Type A, influenza Type B, or influenza Type C. It will be understood by one of ordinary skill in the art that the designation of a virus as a specific Type relates to sequence differences in the virus' matrix (M1) protein or nucleoprotein (NP). Type A influenza virus is further divided into Group 1 and Group 2, which are further divided into subtypes, which refers to a classification of a virus based on the sequence of its hemagglutinin (HA) protein. Examples of Type A influenza subtypes include, but are not limited to, H1, H2, H3, H4, H5, H6, H7, H8, H9, H10, H11, H12, H13, H14, H15, and H16. Group 1 influenza subtypes are H1, H2, H5, H7, and H9. Group 2 influenza subtypes are H3, H4, H6, H8, H10, H11, H12, H13, H14, H15, and H16. The term “strain” refers to viruses within a subtype that have genetic variations (e.g., SNPs) from one another.

The influenza virus is an RNA enveloped virus with a particle size of about 50-125 nm in diameter (see, e.g., Vajda et al., J ChromatogrA 1465:117-125 (2016)). It consists basically of an internal nucleocapsid or core of ribonucleic acid (RNA) associated with nucleoprotein, surrounded by a viral envelope with a lipid bilayer structure and external glycoproteins. Influenza virus comprises two surface antigens, the glycoproteins neuraminidase (NA) and hemagglutinin (HA).

The influenza virus HA protein mediates receptor binding and membrane fusion functions that are essential for virus entry into host cells. Full length HA sequences exhibit significant sequence heterogeneity even within a Group (e.g., Group 1 and Group 2). The HA protein comprises HA1 and HA2 subunits that arise from the proteolytic cleavage of the HA0 precursor. The HA1 “head” is highly variable, immunodominant, and critical for binding sialic acid. Sialic acid is considered the receptor. The HA “stem” or “stalk” contains the fusion domain and comprises the HA2 subunit and an N-terminal portion comprising a 40-50 AA portion of HA1. HA stabilized-stem (referred to herein as “HA-ss”) glycoproteins lacking at least a portion of the HA head region were developed as a candidate for a vaccine antigen, e.g., as described in Yassine et al., Nat Med 21(9):1065-1070 (2015). In embodiments, the HA-ss comprises less than 50%, less than 45%, less than 40%, less than 35%, less than 30%, less than 25%, less than 20%, or less than 15% of the HA head region. In embodiments, the HA-ss comprises a short linker that replaces at least a portion of the HA head region. In embodiments, the HA-ss comprises a thermostable HIV-1 glycoprotein 41 (gp41) trimerization domain that replaces the membrane-distal region of HA2. In embodiments, the HA-ss comprises a truncated membrane-distal HAT and/or HA2 region of the stem. In embodiments, the HA-ss comprises one or more mutations in the linker between HA and gp41. In embodiments, the HA-ss comprises a short glycine-rich linker that replaces the membrane-distal region of HA2. In embodiments, the HA-ss comprises a methionine-leucine hydrophobic pair that replaces an internal salt bridge in the HA2 region. The HA-ss has a conformation similar to the pre-fusion conformation of a full-length, wild-type HA protein.

When the HA-ss is joined to a stabilizing amino acid sequence, e.g., a monomeric subunit protein, such as ferritin, multiple copies of the resulting fusion protein form defined nanoparticles, the surfaces of which displays trimers of the HA-ss. The HA-ss nanoparticle is referred to herein as “HA-ss-np.” In embodiments, the HA-ss is fused from its C-terminus to a ferritin. In embodiments, the ferritin is a bacterial ferritin, a plant ferritin, an algal ferritin, an insect ferritin, a fungal ferritin, or a mammalian ferritin. In embodiments, the ferritin is Helicobacter pylori ferritin. Native H. pylori ferritin proteins spontaneously self-assemble into a nanoparticle of 24 monomer units. In embodiments, the HA-ss-np comprises 24 HA-ss-ferritin fusion proteins. In embodiments, the 24 HA-ss-ferritin fusion proteins self-assemble such that the HA-ss trimerize, resulting in eight trimers displayed on the surface of the nanoparticle. In embodiments, the HA-ss-np is about 1 nm to about 50 nm in diameter, or about 2 nm to about 40 nm in diameter, or about 3 nm to about 35 nm in diameter, or about 5 nm to about 30 nm in diameter, or about 25 nm in diameter, or about 15 nm in diameter, or about 10 nm in diameter. Examples of HA-ss-np are further described, e.g., in Yassine et al., Nat Med 21:1065-1070 (2015); Gallagher et al., Vaccines 6(2):31 (2018); Kanekiyo et al., Nature 499(7456):102-106 (2013); WO2013/044203; WO2015/183969; WO2016/109792; and WO2018/045308.

In embodiments, the antigen of the vaccine composition comprises an HA protein or fragment thereof. In embodiments, the antigen comprises an HA stem region. In embodiments, the antigen comprises HA-ss. In embodiments, the antigen comprises HA-ss-np. In embodiments, the HA-ss-np comprises an HA-ss joined to a ferritin. In embodiments, the ferritin is Helicobacter pylori ferritin. In embodiments, the HA-ss-np comprises any of SEQ ID NOs:5-8. In embodiments, the HA-ss-np comprises a sequence having at least 70%, at least 75%, at least 80%, at least 85%, at least 90%, at least 91%, at least 92%, at least 93%, at least 94%, at least 95%, at least 96%, at least 97%, at least 98%, or at least 99% identity to any one of SEQ ID NOs:1-4.

In embodiments, the HA-ss-np comprises a ferritin subunit having at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO:13 or 14. In embodiments, the HA-ss is linked to the ferritin via a linker of about 1 to about 20 amino acids, or about 1 to about 15 amino acids, or about 1 to about 10 amino acids, or about 1 to about 5 amino acids. In embodiments, the HA-ss is linked to the ferritin via a linker of 1, 2, 3, 4, 5, 6, 7, 8, 9, 10, 11, 12, 13, 14, 15, 16, 17, 18, 19, 20, or more than 20 amino acids.

In embodiments, the HA-ss-np comprises at least 25 contiguous amino acids from an HA of a Type A influenza virus. In embodiments, the HA-ss-np comprises at least 25 contiguous amino acids from an HA of a Type A/Group 1 influenza virus. In embodiments, the Type A/Group 1 influenza virus is influenza subtype H1. In embodiments, the HA-ss-np comprises at least 25 contiguous amino acids from an HA of a Type A/Group 2 influenza virus. In embodiments, the Type A/Group 2 influenza virus is influenza subtype H10. In embodiments, the HA-ss-np comprises an HA-ss having at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO:11 or 12.

In embodiments, the disclosure provides a composition comprising a full length HA protein or fragment thereof, e.g., an HA-ss. In embodiments, the full length HA protein is immunogenic. In embodiments, the HA-ss is immunogenic. In embodiments, the HA-ss-np is immunogenic. In embodiments, the HA protein and/or HA-ss comprises an immunogenic epitope. In embodiments, an epitope elicits a neutralizing antibody response against a homologous influenza strain, i.e., a strain from which the HA is derived. In embodiments, the epitope elicits a neutralizing antibody response against a heterologous influenza strain, i.e., a strain having a non-identical HA as the immunogenic HA. In embodiments, the epitope elicits broadly neutralizing antibodies. In embodiments, a broadly neutralizing antibody elicited against an HA protein, HA-ss, or HA-ss-np from an influenza virus Type (e.g., Type A) is capable of neutralizing influenza virus from a different influenza Type. In embodiments, a broadly neutralizing antibody elicited against an HA protein, HA-ss, or HA-ss-np from a Type A influenza virus is capable of neutralizing Type B or Type C influenza virus. In embodiments, a broadly neutralizing antibody elicited against an HA protein, HA-ss, or HA-ss-np from a Group 1 influenza virus is capable of neutralizing a Group 2 influenza virus. In embodiments, a broadly neutralizing antibody elicited against an HA protein, HA-ss, or HA-ss-np from one sub-type or strain of virus is capable of neutralizing another sub-type or strain of virus. For example, broadly neutralizing antibodies elicited against an HA protein, HA-ss-, or HA-ss-np from an H1 influenza virus may neutralize viruses from one or more sub-types selected from H2, H3, H4, H5, H6, H7, H8, H9, H10, H11, H12, H13, H14, H15, and H16.

In embodiments, the invention provides a vaccine composition comprising HA-ss-np; an aluminum salt; a synthetic oligodeoxynucleotide containing at least one CpG motif (CpG ODN); and a binding modulator, wherein the antigen is not substantially adsorbed to the aluminum salt, and wherein at least a portion of the CpG ODN is adsorbed to the aluminum salt in the composition.

### Adjuvants

In embodiments, the vaccine composition comprises an adjuvant. Adjuvants are substances that may increase and/or modulate the immune response to a vaccine, e.g., by stimulating the immune system to respond more vigorously to a vaccine and thus providing increased immunity to a particular disease. In general, adjuvants provide stimulation using a broad range of mechanisms. Some adjuvants accomplish this task by mimicking specific sets of evolutionarily conserved molecules, termed pathogen-associated molecular patterns, which include liposomes, lipopolysaccharides, molecular cages for antigens, components of bacterial cell walls, and endocytosed nucleic acids, e.g., double-stranded RNA, double-stranded RNA, single-stranded DNA, and methylated or unmethylated CpG dinucleotide-containing DNA. Because immune systems have evolved to recognize these antigenic moieties, the presence of an adjuvant in conjunction with a vaccine can greatly increase the immune response to the antigen by augmenting the activities of dendritic cells, lymphocytes, and macrophages by mimicking a natural infection. Other adjuvants assist immunity by physical means and signaling mechanisms combined, including metal salts such as aluminum hydroxide (Al(OH)₃). Non-limiting examples of vaccine adjuvants include inorganic compounds such as potassium salts, aluminum salts, and calcium phosphate hydroxide; oils such as paraffin oil, squalene oil, monophosphoryl lipid A (MPLA), or glucopyranosyl lipid (GLA); bacterial products, plant carbohydrates, plant saponins, e.g., from the soap bark tree Quillaja saponaria; cytokines; and organic small molecules such as resiquimod. Non-limiting examples of commercially available adjuvants or their formulations include Alhydrogel® (aluminum hydroxide), AdjuPhos® (aluminum phosphate), Advax® (inulin), AddaVax™ (squalene-based), Monophsophoryl Lipid A (MPLA), PolyI:PolyC (Hiltonol®), Rehydragel® (aluminum hydroxide), Rehydraphos™ (amorphous aluminum hydroxyphosphate), Quil-A® (saponin), and the Sigma Adjuvant System (SAS®).

In embodiments, the vaccine composition provided herein comprises an aluminum salt. In embodiments, the vaccine composition provided herein comprises a synthetic oligodeoxynucleotide containing at least one CpG motif (CpG ODN).

### Aluminum Salt

In embodiments, the aluminum salt comprises potassium aluminum sulfate, aluminum hydroxide, aluminum oxyhydroxide, aluminum phosphate, aluminum oxide hydroxide, amorphous aluminum hydroxyphosphate sulfate (AAHS), or combination thereof. In embodiments, the aluminum salt is in the form of a sol or gel. In embodiments, the aluminum salt comprises aluminum hydroxide. In embodiments, the aluminum hydroxide is in the form of a sol-gel. In embodiments, the aluminum hydroxide is in the form of a wet gel suspension. Exemplary aluminum salts include, but are not limited to, the commercially available aluminum hydroxides Alhydrogel®, Rehydragel™, and Imject™, and the commercially available aluminum phosphate gel Adju-Phos®. In embodiments, the aluminum salt comprises Alhydrogel®. In embodiments, aluminum hydroxide consists of fine crystalline particles that are comprised of corrugated layers of aluminum oxyhydroxide, wherein the aluminum atom is coordinated by four oxygen atoms and two hydroxyl groups, and the layers are held together by hydrogen bonds. In embodiments where aluminum hydroxide particles are in aqueous solution, the particles form aggregates from about 0.5 μm to about 15 μm in diameter, e.g., about 1 μm to about 10 μm, or about 0.95 μm to about 7.5 pin, or about 2.5 μm to about 5 μm in diameter. In embodiments, the aggregates exhibit a size distribution with a median aggregate size of about 0.5 μm to about 15 μm in diameter, e.g., about 1 μm to about 10 μm, or about 0.95 m to about 7.5 μm, or about 2.5 μm to about 5 μm in diameter. In embodiments, aluminum hydroxide particles have a point of 0 charge (i.e., the pH at which the charge on the particle is 0) at about pH 10.5 to 11.5, e.g., about pH 11. In embodiments, the aluminum hydroxide is positively charged at physiological pH, e.g., about pH 6.5 to 8.5, or about pH 7 to 8, or about pH 7.2 to 7.8, or about pH 7.4 to 7.7, or about pH 7.5 to 7.6. In embodiments, the aluminum hydroxide is capable of adsorbing acidic components (e.g., proteins, polynucleotides, or other small molecules) by electrostatic attraction. See, e.g., Shardlow et al., Front Chem 4:48 (2017) and Watkingson et al., Clin Vaccine Immunol 20(11):1659-1668 (2013).

In general, aluminum salts, e.g., aluminum hydroxide, boost the antibody-mediated (Th2) immune response to an antigen. The mechanism of action of aluminum salts to boost the immune response typically involves adsorption of antigens onto the aluminum salt particles. When the antigen-adsorbed aluminum salt particles are administered into the host subject, the aluminum salt promotes a localized immune response directed toward the antigen, including secretion of antigen-specific antibodies, e.g., via aggregate formation to enable more prolonged release of the antigens, formation of particle structures that promote phagocytosis of antigens by antigen-presenting cells (APCs), and induction of local inflammation via the NLRP3 inflammasome. NLRP3 inflammasome activation induces the secretion of IL-1β and IL-18 by dendritic cells and the differentiation of TH2 cells, promoting the activation of B cells and the production of antibodies. Mechanisms of aluminum salts are further described in, e.g., Martiñón et al., J Immunol Res vol. 2019, Article ID 3974127 (2019). If the antigen is not completely adsorbed onto the aluminum salt particles, the antigen and aluminum salt may not be directed to the same cellular location upon administration. Consequently, the immune response triggered by the aluminum salt is not expected to be specific towards the antigen. Thus, it was known in the field that antigens should bind to or be adsorbed to adsorption sites in the aluminum salt in order to promote an antigen-specific immune response.

It was therefore surprisingly discovered that the vaccine compositions disclosed herein, in which the antigen, e.g., HA-ss-np, is not substantially adsorbed to the aluminum salt, e.g., aluminum hydroxide, were highly effective in inducing an antigen-specific immune response. In embodiments, an antigen that is “not substantially adsorbed” to the aluminum salt in a composition means that less than 10%, less than 8%, less than 5%, less than 3%, less than 1%, less than 0.75%, less than 0.5%, less than 0.25%, less than 0.1%, less than 0.075%, less than 0.05%, less than 0.025%, less than 0.01%, or no detectable amount of the antigen in the vaccine composition is adsorbed to aluminum salt, e.g., aluminum hydroxide. In embodiments, less than 10%, less than 9%, less than 8%, less than 7%, less than 6%, less than 5%, less than 4%, less than 3%, less than 2%, less than 1%, less than 0.75%, less than 0.5%, less than 0.25%, less than 0.1%, less than 0.075%, less than 0.05%, less than 0.025%, or less than 0.01% of the adsorption capacity of the aluminum salt, e.g., aluminum hydroxide, is bound to the antigen in the vaccine composition. In embodiments, none or substantially none of the adsorption capacity in the aluminum salt, e.g., aluminum hydroxide, is bound to the antigen in the vaccine composition. In embodiments, adsorption capacity of the aluminum salt, e.g., aluminum hydroxide, is available to a further adjuvant in the vaccine composition. In embodiments, a further adjuvant in the vaccine composition occupies at least some, a substantial amount or all of the adsorption capacity of the aluminum salt, e.g., aluminum hydroxide. In embodiments, the further adjuvant comprises a synthetic oligodeoxynucleotide containing at least one CpG motif (CpG ODN). In embodiments, the CpG ODN is CPG 7909.

In embodiments, the vaccine composition comprises HA-ss-np; CpG ODN; a phosphate salt; and
about 0.01% weight/volume (w/v) to about 5% w/v of aluminum hydroxide,
about 0.01% w/v to about 4% w/v of aluminum hydroxide,
about 0.01% w/v to about 3% w/v of aluminum hydroxide,
about 0.010% w/v to about 2% w/v of aluminum hydroxide,
about 0.010% w/v to about 1% w/v of aluminum hydroxide,
about 0.02% w/v to about 0.5% w/v of aluminum hydroxide,
about 0.02% w/v to about 0.2% w/v of aluminum hydroxide,
about 0.02% w/v to about 0.15% w/v of aluminum hydroxide,
about 0.02% w/v to about 0.1% w/v of aluminum hydroxide,
about 0.03% w/v to about 0.09% w/v of aluminum hydroxide,
about 0.04% w/v to about 0.08% w/v of aluminum hydroxide,
about 0.05% w/v to about 0.07% w/v of aluminum hydroxide, or
about 0.06% w/v of aluminum hydroxide,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an HA-ss-np from a Group 1 influenza virus (A1-ss-np) and an HA-ss-np from a Group 2 influenza virus (A2-ss-np). In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises HA-ss-np; CpG ODN; a phosphate salt; and about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.1% w/v, about 0.15% w/v, about 0.2% w/v, about 0.5% w/v, about 1% w/v, about 2% w/v, about 3% w/v, about 4% w/v, or about 5% w/v of aluminum hydroxide, wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises HA-ss-np; CpG ODN; a phosphate salt; and
about 0.1 mg/mL to about 50 mg/mL of aluminum as aluminum hydroxide,
about 0.1 mg/mL to about 40 mg/mL of aluminum as aluminum hydroxide,
about 0.1 mg/mL to about 30 mg/mL of aluminum as aluminum hydroxide,
about 0.1 mg/mL to about 20 mg/mL of aluminum as aluminum hydroxide,
about 0.1 mg/mL to about 10 mg/mL of aluminum as aluminum hydroxide,
about 0.2 mg/mL to about 5 mg/mL of aluminum as aluminum hydroxide,
about 0.2 mg/mL to about 2 mg/mL of aluminum as aluminum hydroxide,
about 0.2 mg/mL to about 1.5 mg/mL of aluminum as aluminum hydroxide,
about 0.2 mg/mL to about 1 mg/mL of aluminum as aluminum hydroxide,
about 0.3 mg/mL to about 0.9 mg/mL of aluminum as aluminum hydroxide,
about 0.4 mg/mL to about 0.8 mg/mL of aluminum as aluminum hydroxide,
about 0.5 mg/mL to about 0.7 mg/mL of aluminum as aluminum hydroxide, or
about 0.6 mg/mL of aluminum as aluminum hydroxide,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises HA-ss-np; CpG ODN; a phosphate salt; and about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.5 mg/mL, about 2.0 mg/mL, about 3.0 mg/mL, about 4.0 mg/mL, about 5.0 mg/mL, about 10 mg/mL, about 20 mg/mL, about 30 mg/mL, about 40 mg/mL, or about 50 mg/mL of aluminum as the aluminum hydroxide, wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

### CpG ODN

CpG ODNs are short single-stranded synthetic DNA molecules that contain at least one CpG motif, i.e., a methylated or unmethylated cytosine triphosphate deoxynucleotide followed by a guanine triphosphate deoxynucleotide. In embodiments, the CpG ODN comprises a phosphodiester backbone. In embodiments, the CpG ODN comprises at least one methylated CpG motif. In embodiments, the CpG ODN comprises at least one unmethylated CpG motif. In embodiments, the CpG ODN is unmethylated. In embodiments, the CpG ODN comprises a phosphorothioate backbone. In general, CpG ODNs trigger cells that express Toll-like receptor 9 (TLR9) to mount an immune response characterized by the production of Th1 and proinflammatory cytokines, improving the function of APCs and boosting the generation of antigen-specific immune responses. At least four classes of CpG ODN have been described. D-type (also referred to as A-class) CpG ODNs comprise a mixed phosphodiester/phosphorothioate backbone, a single CpG motif, a palindrome formed by the CpG flanking region, and a poly G tail at the 3′ end. K-type (also referred to as B-class) CpG ODNs comprise a phosphorothioate backbone and multiple CpG motifs, with the 5′ motif being the most stimulatory. C-type CpG ODNs comprise a phosphorothioate backbone, multiple CpG motifs, a TCG dimer at the 5′ end, and a CpG motif embedded in a central palindrome. P-type CpG ODNs comprise a phosphorothioate backbone, multiple CpG motifs, and two palindromes. CpG ODNs are further described in, e.g., Bode et al., Expert Rev Vaccines 10(4):499-511 (2011). Exemplary CpG ODNs include, but are not limited to, CPG 7909, CPG 1826, and CPG 1808. In embodiments, the CpG ODN of the vaccine composition is an A-class CpG ODN. In embodiments, the CpG ODN of the vaccine composition is a B-class CpG ODN. In embodiments, the CpG ODN of the vaccine composition is a C-type CpG ODN. In embodiments, the CpG ODN of the vaccine composition is a P-type CpG ODN. In embodiments, the CpG ODN is CPG 7909, CPG 1826, or CPG 1808.

In embodiments, the CpG ODN is CPG 7909. CPG 7909 is a B-class CpG ODN comprising 24 nucleotides linked with a phosphorothioate backbone and is a ligand for human TLR9. CPG 7909 has the following chemical structure:

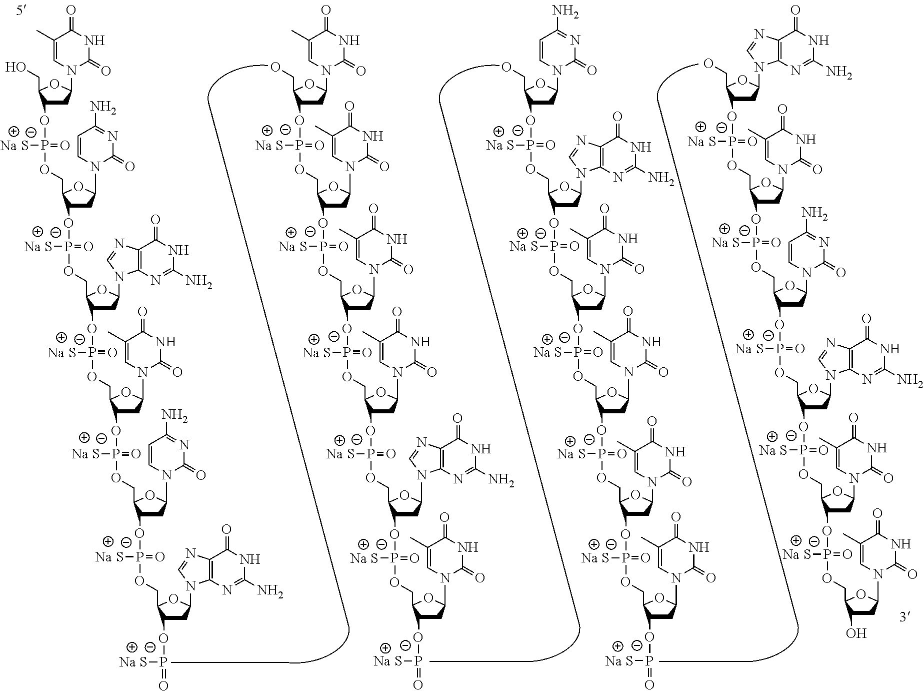

and the sequence: 5′-TCG TCG TTT TGT CGT TTT GTC GTT-3 (SEQ ID NO: 16), wherein the nucleotide backbone is a phosphorothioate backbone.

In embodiments, the CpG ODN is CPG 1826. CPG 1826 comprises 20 nucleotides linked with a phosphorothioate backbone and is a ligand for murine TLR9. In embodiments, the biological activity of CPG 1826 in mice mimics the biological activity of CPG 7909 in humans.

In embodiments, the vaccine composition comprises HA-ss-np; aluminum hydroxide; a phosphate salt; and
about 0.01% w/v to about 0.5% w/v of CpG ODN,
about 0.02% w/v to about 0.5% w/v of CpG ODN,
about 0.03% w/v to about 0.4% w/v of CpG ODN,
about 0.04% w/v to about 0.3% w/v of CpG ODN,
about 0.05% w/v to about 0.2% w/v of CpG ODN,
about 0.05% w/v to about 0.1% w/v of CpG ODN,
about 0.05% w/v to about 0.08% w/v of CpG ODN,
about 0.06% w/v to about 0.18% w/v of CpG ODN,
about 0.07% w/v to about 0.15% w/v of CpG ODN,
about 0.08% w/v to about 0.14% w/v of CpG ODN,
about 0.09% w/v to about 0.12% w/v of CpG ODN,
about 0.09% w/v to about 0.11% w/v of CpG ODN,
about 0.010% w/v to about 0.12% w/v of CpG ODN,
about 0.05% w/v of CpG ODN, or
about 0.10% w/v of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises HA-ss-np; aluminum hydroxide; a phosphate salt; and about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.1% w/v, about 0.12% w/v, about 0.15% w/v, about 0.17% w/v, about 0.2% w/v, about 0.25% w/v, about 0.3% w/v, about 0.35% w/v, about 0.4% w/v, about 0.45% w/v, or about 0.5% w/v of CpG ODN, wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises HA-ss-np; aluminum hydroxide; a phosphate salt; and
about 0.1 mg/mL to about 5 mg/mL of CpG ODN,
about 0.2 mg/mL to about 5 mg/mL of CpG ODN,
about 0.3 mg/mL to about 4 mg/mL of CpG ODN,
about 0.4 mg/mL to about 3 mg/mL of CpG ODN,
about 0.5 mg/mL to about 2 mg/mL of CpG ODN,
about 0.5 mg/mL to about 1 mg/mL of CpG ODN,
about 0.5 mg/mL to about 0.8 mg/mL of CpG ODN,
about 0.6 mg/mL to about 1.8 mg/mL of CpG ODN,
about 0.7 mg/mL to about 1.5 mg/mL of CpG ODN,
about 0.8 mg/mL to about 1.4 mg/mL of CpG ODN,
about 0.9 mg/mL to about 1.2 mg/mL of CpG ODN,
about 0.9 mg/mL to about 1.1 mg/mL of CpG ODN,
about 0.1 mg/mL to about 1.2 mg/mL of CpG ODN,
about 0.5 mg/mL of CpG ODN, or
about 1 mg/mL of CpG ODN;
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises HA-ss-np; aluminum hydroxide; a phosphate salt; and about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.2 mg/mL, about 1.5 mg/mL, about 1.7 mg/mL, about 2.0 mg/mL, about 2.5 mg/mL, about 3.0 mg/mL, about 3.5 mg/mL, about 4.0 mg/mL, about 4.5 mg/mL, or about 5.0 mg/mL of CpG ODN, wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the HA-ss-np antigen in the vaccine composition is not substantially adsorbed to the aluminum hydroxide. In embodiments, the HA-ss-np in the vaccine composition is not substantially adsorbed to the aluminum hydroxide, and at least a portion of the CpG ODN in the vaccine composition is adsorbed to the aluminum hydroxide. In embodiments, at least 80%, at least 85%, at least 90%, at least 91%, at least 92%, at least 93%, at least 94%, at least 95%, at least 96%, at least 97%, at least 98%, at least 99%, or about 100% of the adsorption sites in the aluminum hydroxide is bound to the CpG ODN. In embodiments, a CpG ODN occupies or binds at least 80%, at least 85%, at least 90%, at least 91%, at least 92%, at least 93%, at least 94%, at least 95%, at least 96%, at least 97%, at least 98%, at least 99%, or about 100% of the adsorption capacity of the aluminum hydroxide. In embodiments, the CpG ODN is CPG 7909.

In embodiments, the CpG ODN is present in excess of the aluminum hydroxide in the vaccine composition. Previous work (e.g., Aebig et al., J Immunol Methods 323(2):139-146 (2007)) has shown that an excess of aluminum hydroxide to CpG ODN is required for maximal binding of the CpG ODN to the aluminum hydroxide. It was therefore unexpected that the vaccine composition of the present invention had higher efficacy when CpG ODN is present in excess of the aluminum hydroxide. In embodiments, the presence of excess CpG ODN, which remains in solution once all the adsorption capacity of the aluminum hydroxide is bound to CpG ODN, improves the antigen-specific immune response to the vaccine composition. In embodiments, the CpG ODN is CPG 7909.

In embodiments, the weight/weight (w/w) ratio of aluminum in the aluminum hydroxide to the CpG ODN in the vaccine composition is about 1:1 to greater than about 1:10. In embodiments, the w/w ratio of aluminum in the aluminum hydroxide to the CpG ODN in the vaccine composition is about 1:1 to about 1:10, about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3, about 1:1 to about 1:2, about 1:1.5 to about 1:1.9, about 1:1.6 to about 1:1.8, about 1:14 to about 1.17, or about 1:1 to about 1:1.5. In embodiments, the w/w ratio of aluminum in the aluminum hydroxide to the CpG ODN in the vaccine composition is about 1:1 to about 1.5. In embodiments, the w/w ratio of aluminum in the aluminum hydroxide to the CpG ODN in the vaccine composition is about 1:1 to about 1.2. In embodiments, the w/w ratio of aluminum in the aluminum hydroxide to the CpG ODN in the vaccine composition is about 1:1, about 1:1.3, about 1:1.5, about 1:1.7, about 1:2, about 1:3, about 1:4, about 1:5, about 1:6, about 1:7, about 1:8, about 1:9, about 1:10, or greater than about 1:10. In embodiments, about 10% to about 100%, or about 15% to about 95%, or about 20% to about 90%, or about 25% to about 85%, or about 30% to about 80%, or about 35% to about 75%, or about 40% to about 70%, or about 45% to about 65%, or about 50% to about 60% of the CpG ODN in the vaccine composition is adsorbed to the aluminum hydroxide. In embodiments, about 10%, about 20%, about 30%, about 40%, about 50%, about 60%, about 70%, about 80%, about 90%, or about 100% of the CpG ODN in the vaccine composition is adsorbed to the aluminum hydroxide. In embodiments, the CpG ODN is CPG 7909. In embodiments, the HA-ss-np antigen is not substantially adsorbed to the aluminum hydroxide.

In embodiments, about 0.5%, 1%, 2%, 3%, 4%, 5%, 6%, 7%, 8%, 9%, %, 20%, 25%, 30%, 35%, 40%, 45%, 50%, 55%, 60%, 65%, 70%, 75%, 80%, 85, 90%, or 95% of the total CpG ODN in the vaccine composition is present in solution, with the remaining CpG ODN in the vaccine composition being adsorbed to the aluminum hydroxide. In embodiments, less than about 0.5%, 1%, 2%, 3%, 4%, 5%, 6%, 7%, 8%, 9%, 10%, 20%, 25%, 30%, 35%, 40%, 4%5% 50%, or 55% of the total CpG ODN in the vaccine composition is present in solution, with the remaining CpG ODN in the vaccine composition being adsorbed to the aluminum hydroxide. In embodiments, greater than about 0.5%, 1%, 2%, 3%, 4%, 5%, 6%, 7%, 8%, 9%, or 10%, of the total CpG ODN in the vaccine composition is present in solution, with the remaining CpG ODN in the vaccine composition being adsorbed to the aluminum hydroxide. In embodiments, the excess CpG ODN in solution in the vaccine composition enhances the antigen-specific immune response. In embodiments, the CpG ODN is CPG 7909. In embodiments, the HA-ss-np antigen is not substantially adsorbed to the aluminum hydroxide.

As described herein, vaccines that utilize aluminum salts, e.g., aluminum hydroxide, generally require complete adsorption of the antigens (i.e., close to or about 100% of the antigens) in the composition to the aluminum salt. Typically, incomplete adsorption of the antigen to the aluminum salt results in weaker vaccine efficacy. It was therefore unexpected that the vaccine compositions described herein, in which the HA-ss-np antigen is not substantially adsorbed to the aluminum hydroxide and at least a portion of the CpG ODN is adsorbed to the aluminum hydroxide, had higher efficacy as compared to a vaccine in which the HA-ss-np is adsorbed to the aluminum hydroxide.

The invention further provides an adjuvant system, in which the CpG ODN fully occupies the adsorption sites on the aluminum salt, e.g., aluminum hydroxide. Any suitable antigen can be added to such an adjuvant system. Because the aluminum salt adsorption capacity is fully occupied by the CpG ODN, the typical processes required to ensure antigen adsorption onto the aluminum salt are advantageously eliminated, thereby simplifying the vaccine manufacture process. In embodiments, the aluminum salt is aluminum hydroxide, and the CpG ODN is CPG 7909. In embodiments, the antigen is HA-ss-np. In embodiments, the HA-ss-np comprises A1-ss-np and A2-ss-np. In embodiments, the adjuvant system further comprises a phosphate salt. In embodiments, the phosphate salt is sodium phosphate monobasic monohydrate. Phosphate salts are further described herein.

In embodiments, the vaccine composition of the invention comprises
HA-ss-np;
a phosphate salt;
about 0.01% w/v to about 1% w/v of aluminum hydroxide; and
about 0.01% w/v to about 0.5% w/v of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.02% w/v to about 0.15% w/v of aluminum hydroxide; and
about 0.010% w/v to about 0.12% w/v of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.04% w/v to about 0.08% w/v of aluminum hydroxide; and
about 0.08% w/v to about 0.15% w/v of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.04% w/v to about 0.08% w/v of aluminum hydroxide; and
about 0.01% w/v to about 0.10% w/v of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.06 w/v of aluminum hydroxide; and
about 0.01% w/v to about 0.5% w/v of CpG ODN,
about 0.01% w/v to about 0.12% w/v of CpG ODN,
about 0.02% w/v to about 0.5% w/v of CpG ODN,
about 0.01% w/v to about 0.1% w/v of CpG ODN,
about 0.05% w/v to about 0.2% w/v of CpG ODN,
about 0.08% w/v to about 0.15% w/v of CpG ODN, or
about 0.06% w/v to about 0.12% w/v of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.01% w/v to about 1% w/v of aluminum hydroxide,
about 0.02% w/v to about 0.15% w/v of aluminum hydroxide,
about 0.04% w/v to about 0.08% w/v of aluminum hydroxide, or
about 0.05% w/v to about 0.07% w/v of aluminum hydroxide; and
about 0.1% w/v of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.010% w/v to about 1% w/v of aluminum hydroxide,
about 0.02% w/v to about 0.15% w/v of aluminum hydroxide,
about 0.04% w/v to about 0.08% w/v of aluminum hydroxide, or
about 0.05% w/v to about 0.07% w/v of aluminum hydroxide; and
about 0.05% w/v of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises HA-ss-np; a phosphate salt; about 0.06% w/v of aluminum hydroxide; and about 0.05% w/v or about 0.1% w/v of CpG ODN,

wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition of the invention comprises
HA-ss-np;
a phosphate salt;
about 0.1 mg/mL to about 10 mg/mL of aluminum as aluminum hydroxide; and
about 0.1 mg/mL to about 5 mg/mL of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.2 mg/mL to about 1.5 mg/mL of aluminum as aluminum hydroxide; and
about 0.1 mg/mL to about 1.2 mg/mL of CpG ODN,
wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.4 mg/mL to about 0.8 mg/mL of aluminum as aluminum hydroxide; and
about 0.8 mg/mL to about 1.5 mg/mL of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.4 mg/mL to about 0.8 mg/mL of aluminum as aluminum hydroxide; and
about 0.1 mg/mL to about 1.0 mg/mL of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.6 mg/mL of aluminum as aluminum hydroxide; and
about 0.1 mg/mL to about 5 mg/mL of CpG ODN,
about 0.1 mg/mL to about 1.2 mg/mL of CpG ODN,
about 0.2 mg/mL to about 5 mg/mL of CpG ODN,
about 0.1 mg/mL to about 1 mg/mL of CpG ODN,
about 0.5 mg/mL to about 2 mg/mL of CpG ODN,
about 0.8 mg/mL to about 1.5 mg/mL of CpG ODN, or
about 0.6 mg/mL to about 1.2 mg/mL of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.1 mg/mL to about 1 mg/mL of aluminum as aluminum hydroxide,
about 0.2 mg/mL to about 1.5 mg/mL of aluminum as aluminum hydroxide,
about 0.4 mg/mL to about 0.8 mg/mL of aluminum as aluminum hydroxide, or
about 0.5 mg/mL to about 0.7 mg/mL of aluminum as aluminum hydroxide; and
about 1 mg/mL of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
a phosphate salt;
about 0.1 mg/mL to about 1 mg/mL of aluminum as aluminum hydroxide,
about 0.2 mg/mL to about 1.5 mg/mL of aluminum as aluminum hydroxide,
about 0.4 mg/mL to about 0.8 mg/mL of aluminum as aluminum hydroxide, or
about 0.5 mg/mL to about 0.7 mg/mL of aluminum as aluminum hydroxide; and
about 0.5 mg/mL of CpG ODN,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises HA-ss-np; a phosphate salt; about 0.6 mg/mL of aluminum as aluminum hydroxide; and about 0.5 mg/mL or about 1 mg/mL of CpG ODN, wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

### Binding Modulator

In embodiments, the vaccine composition provided herein comprises HA-ss-np, aluminum hydroxide, CpG ODN, and a binding modulator. Adsorption of compounds such as CpG ODN onto aluminum salts can be modulated via mechanisms including but not limited to hydrophobic or hydrogen bonding, electrostatic, and ligand exchange interactions. In the ligand exchange mechanism, a ligand of the aluminum salt (e.g., a hydroxyl group of aluminum hydroxide or a phosphate group of aluminum phosphate gel) can exchange with an anion of the binding modulator, which affects the binding capacity of the aluminum salt. In embodiments, the binding modulator comprises a phosphate salt, a fluoride salt, an acetate salt, or combination thereof. In embodiments, the binding modulator is a pH adjustment agent. In embodiments, the aluminum salt is aluminum hydroxide, and the binding modulator is a phosphate salt. In embodiments, the phosphate salt comprises a sodium cation. In embodiments, the phosphate salt comprises a calcium cation. In embodiments, the phosphate salt is sodium phosphate monobasic monohydrate (NaH₂PO₄·H₂O). In embodiments, the phosphate salt is sodium phosphate dibasic. In embodiments, the phosphate salt is sodium phosphate tribasic.

Phosphate salt has been shown to inhibit binding of CpG ODN to aluminum hydroxide. See, e.g., Aebig et al., J Immunol Methods 323(2):139-146 (2007). It was therefore unexpected that the vaccine compositions of the present invention had significant binding between aluminum hydroxide and CpG ODN even in the presence of phosphate salt.

In embodiments, the vaccine composition comprises HA-ss-np; aluminum hydroxide; CpG ODN; and
about 0.1 mM to: about 100 mM, about 90 mM, about 80 mM, about 70 mM, about 60 mM, about 50 mM, about 40 mM, about 30 mM, about 20 mM, or about 10 mM of phosphate salt,
about 0.2 mM to: about 50 mM, about 40 mM, about 30 mM, about 10 mM, about 4 mM, about 1 mM, or about 0.6 mM of phosphate salt,
about 0.3 mM to: about 0.8 mM, about 0.5 mM, or about 0.4 mM of phosphate salt,
about 0.35 mM to: about 0.7 mM or about 0.4 mM of phosphate salt,
about 0.4 mM to about 0.7 mM of phosphate salt,
about 0.5 mM to: about 50 mM, about 40 mM, about 30 mM, about 20 mM, or about 10 mM of phosphate salt,
about 1 mM to: about 50 mM, about 40 mM, about 30 mM, about 20 mM, or about 10 mM of phosphate salt,
about 2 mM to: about 50 mM, about 40 mM, about 30 mM, about 20 mM, or about 10 mM of phosphate salt,
about 5 mM to: about 50 mM, about 40 mM, about 30 mM, about 20 mM, or about 10 mM of phosphate salt, or
about 0.35 mM, about 0.4 mM, about 0.5 mM, about 1 mM, about 2 mM, about 5 mM, about 7 mM, about 10 mM, about 12 mM, about 15 mM, about 17 mM, about 20 mM, about 22 mM, about 25 mM, about 27 mM, about 30 mM, about 32 mM, about 35 mM, about 37 mM, about 40 mM, about 42 mM, about 45 mM, about 47 mM, or about 50 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises HA-ss-np; aluminum hydroxide; CpG ODN; and about 0.3 mM, about 0.35 mM, about 0.4 mM, about 0.45 mM, about 0.5 mM, about 0.55 mM, about 0.6 mM, about 0.65 mM, about 0.7 mM, about 1 mM, about 2 mM, about 5 mM, about 7 mM, about 10 mM, about 12 mM, about 15 mM, about 17 mM, about 20 mM, about 22 mM, about 25 mM, about 27 mM, about 30 mM, about 32 mM, about 35 mM, about 37 mM, about 40 mM, about 42 mM, about 45 mM, about 47 mM, or about 50 mM of phosphate salt, wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, pH of the vaccine composition modulates ligand exchange of the aluminum hydroxide. In embodiments, pH of the vaccine composition modulates adsorption of the CpG ODN to the aluminum hydroxide. For example, aluminum hydroxide has an isoelectric point of 11. Thus, under physiological pH conditions, aluminum hydroxide is highly positive charged and therefore binds a negatively charged protein or oligonucleotide, e.g., CpG ODN. In embodiments, pH of the vaccine composition modulates the amount of adsorption of CpG ODN onto the aluminum hydroxide. In embodiments, the pH of the vaccine composition is about 5 to about 9, about 5.5 to about 8.7, about 6 to about 8.5, about 6.2 to about 8.2, about 6.5 to about 8, about 6.7 to about 7.7, about 6.8 to about 8, about 7 to about 7.9, about 7 to about 7.4, about 7.2 to about 7.8, about 7.3 to about 7.6, or about 7.4 to about 7.5. In embodiments, the pH of the vaccine composition is about 5, about 5.5, about 6, about 6.5, about 6.6, about 6.7, about 6.8, about 6.9, about 7, about 7.1, about 7.2, about 7.3, about 7.4, about 7.5, about 7.6, about 7.7, about 7.8, about 7.9, or about 8.

In embodiments, the vaccine composition of the invention comprises
HA-ss-np,
about 0.01% w/v to about 1% w/v of aluminum hydroxide,
about 0.01% w/v to about 0.5% w/v of CpG ODN, and
about 0.1 mM to about 50 mM, or about 0.5 mM to about 20 mM, or about 1 mM to about 15 mM, or about 2 mM to about 10 mM, or about 0.2 mM to about 4 mM, or about 0.2 mM to about 1 mM, or about 0.3 mM to about 0.7 mM, or about 0.3 mM to about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np,
about 0.02% w/v to about 0.15% w/v of aluminum hydroxide,
about 0.05% w/v to about 0.2% w/v of CpG ODN, and
about 0.1 mM to about 50 mM, or about 0.5 mM to about 20 mM, or about 1 mM to about 15 mM, or about 2 mM to about 10 mM, or about 0.2 mM to about 4 mM, or about 0.2 mM to about 1 mM, or about 0.3 mM to about 0.7 mM, or about 0.3 mM to about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np,
about 0.04% w/v to about 0.08% w/v of aluminum hydroxide,
about 0.08% w/v to about 0.15% w/v of CpG ODN, and
about 0.1 mM to about 50 mM, or about 0.5 mM to about 20 mM, or about 1 mM to about 15 mM, or about 2 mM to about 10 mM, or about 0.2 mM to about 4 mM, or about 0.2 mM to about 1 mM, or about 0.3 mM to about 0.7 mM, or about 0.3 mM to about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np,
about 0.05% w/v to about 0.07% w/v of aluminum hydroxide,
about 0.06% w/v to about 0.12% w/v of CpG ODN, and
about 0.1 mM to about 50 mM, or about 0.5 mM to about 20 mM, or about 1 mM to about 15 mM, or about 2 mM to about 10 mM, or about 0.2 mM to about 4 mM, or about 0.2 mM to about 1 mM, or about 0.3 mM to about 0.7 mM, or about 0.3 mM to about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition of the invention comprises
HA-ss-np,
about 0.1 mg/mL to about 10 mg/mL of aluminum as aluminum hydroxide,
about 0.1 mg/mL to about 5 mg/mL of CpG ODN, and
about 0.1 mM to about 50 mM, or about 0.5 mM to about 20 mM, or about 1 mM to about 15 mM, or about 2 mM to about 10 mM, or about 0.2 mM to about 4 mM, or about 0.2 mM to about 1 mM, or about 0.3 mM to about 0.7 mM, or about 0.3 mM to about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np,
about 0.2 mg/mL to about 1.5 mg/mL of aluminum as aluminum hydroxide,
about 0.1 mg/mL to about 1.2 mg/mL of CpG ODN, and
about 0.1 mM to about 50 mM, or about 0.5 mM to about 20 mM, or about 1 mM to about 15 mM, or about 2 mM to about 10 mM, or about 0.2 mM to about 4 mM, or about 0.2 mM to about 1 mM, or about 0.3 mM to about 0.7 mM, or about 0.3 mM to about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np,
about 0.4 mg/mL to about 0.8 mg/mL of aluminum as aluminum hydroxide,
about 0.8 mg/mL to about 1.5 mg/mL of CpG ODN, and
about 0.1 mM to about 50 mM, or about 0.5 mM to about 20 mM, or about 1 mM to about 15 mM, or about 2 mM to about 10 mM, or about 0.2 mM to about 4 mM, or about 0.2 mM to about 1 mM, or about 0.3 mM to about 0.7 mM, or about 0.3 mM to about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np,
about 0.5 mg/mL to about 0.7 mg/mL of aluminum as aluminum hydroxide,
about 0.6 mg/mL to about 1.2 mg/mL of CpG ODN, and
about 0.1 mM to about 50 mM, or about 0.5 mM to about 20 mM, or about 1 mM to about 15 mM, or about 2 mM to about 10 mM, or about 0.2 mM to about 4 mM, or about 0.2 mM to about 1 mM, or about 0.3 mM to about 0.7 mM, or about 0.3 mM to about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.010% w/v to about 1% w/v of aluminum hydroxide,
about 0.02% w/v to about 0.15% w/v of aluminum hydroxide,
about 0.04% w/v to about 0.08% w/v of aluminum hydroxide,
about 0.05% w/v to about 0.07% w/v of aluminum hydroxide, or
about 0.06% w/v of aluminum hydroxide;
about 0.10% w/v of CpG ODN; and
about 0.36 mM of the phosphate salt,
wherein the HA-ss-np is not substantially adsorbed to the aluminum hydroxide, and
   wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.1 mg/mL to about 10 mg/mL of aluminum as aluminum hydroxide,
about 0.2 mg/mL to about 1.5 mg/mL of aluminum as aluminum hydroxide,
about 0.4 mg/mL to about 0.8 mg/mL of aluminum as aluminum hydroxide,
about 0.5 mg/mL to about 0.7 mg/mL of aluminum as aluminum hydroxide, or
about 0.6 mg/mL of aluminum as aluminum hydroxide;
about 1 mg/mL of CpG ODN; and
about 0.36 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.06 w/v of aluminum hydroxide;
about 0.01% w/v to about 0.5% w/v of CpG ODN,
about 0.010% w/v to about 0.12% w/v of CpG ODN,
about 0.08% w/v to about 0.15% w/v of CpG ODN,
about 0.06% w/v to about 0.12% w/v of CpG ODN, or
about 0.10% w/v of CpG ODN; and
about 0.36 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.6 mg/mL of aluminum as aluminum hydroxide;
about 0.1 mg/mL to about 5 mg/mL of CpG ODN,
about 0.1 mg/mL to about 1.2 mg/mL of CpG ODN,
about 0.8 mg/mL to about 1.5 mg/mL of CpG ODN,
about 0.6 mg/mL to about 1.2 mg/mL of CpG ODN, or
about 1.0 mg/mL of CpG ODN; and
about 0.36 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.01% w/v to about 1% w/v of aluminum hydroxide,
about 0.02% w/v to about 0.15% w/v of aluminum hydroxide,
about 0.04% w/v to about 0.08% w/v of aluminum hydroxide,
about 0.05% w/v to about 0.07% w/v of aluminum hydroxide, or
about 0.06% w/v of aluminum hydroxide;
about 0.05% w/v of CpG ODN; and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.1 mg/mL to about 10 mg/mL of aluminum as aluminum hydroxide,
about 0.2 mg/mL to about 1.5 mg/mL of aluminum as aluminum hydroxide,
about 0.4 mg/mL to about 0.8 mg/mL of aluminum as aluminum hydroxide,
about 0.5 mg/mL to about 0.7 mg/mL of aluminum as aluminum hydroxide, or
about 0.6 mg/mL of aluminum as aluminum hydroxide;
about 0.5 mg/mL of CpG ODN; and
about 5 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.06 w/v of aluminum hydroxide;
about 0.01% w/v to about 0.5% w/v of CpG ODN,
about 0.010% w/v to about 0.10% w/v of CpG ODN,
about 0.02% w/v to about 0.8% w/v of CpG ODN,
about 0.04% w/v to about 0.6% w/v of CpG ODN, or
about 0.05% w/v of CpG ODN; and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.6 mg/mL of aluminum as aluminum hydroxide;
about 0.1 mg/mL to about 5 mg/mL of CpG ODN,
about 0.1 mg/mL to about 1 mg/mL of CpG ODN,
about 0.2 mg/mL to about 0.8 mg/mL of CpG ODN,
about 0.4 mg/mL to about 0.6 mg/mL of CpG ODN, or
about 0.5 mg/mL of CpG ODN; and
about 5 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.06% w/v of aluminum hydroxide;
about 0.1% w/v of CpG ODN; and
about 0.2 mM to about 10 mM of phosphate salt,
about 0.2 mM to about 4 mM of phosphate salt,
about 0.2 mM to about 1 mM of phosphate salt,
about 0.3 mM to about 0.8 mM of phosphate salt,
about 0.3 mM to about 0.5 mM of phosphate salt,
about 0.2 mM to about 0.6 mM of phosphate salt,
about 0.35 mM to about 0.7 mM of phosphate salt,
about 0.4 mM to about 0.7 mM of phosphate salt,
about 0.3 mM to about 0.4 mM of phosphate salt,
about 0.35 mM to about 0.4 mM of phosphate salt,
about 0.36 mM of phosphate salt, or
about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.6 mg/mL of aluminum as aluminum hydroxide;
about 1 mg/mL of CpG ODN; and
about 0.2 mM to about 10 mM of phosphate salt,
about 0.2 mM to about 4 mM of phosphate salt,
about 0.2 mM to about 1 mM of phosphate salt,
about 0.3 mM to about 0.8 mM of phosphate salt,
about 0.3 mM to about 0.5 mM of phosphate salt,
about 0.2 mM to about 0.6 mM of phosphate salt,
about 0.35 mM to about 0.7 mM of phosphate salt,
about 0.4 mM to about 0.7 mM of phosphate salt,
about 0.3 mM to about 0.4 mM of phosphate salt,
about 0.35 mM to about 0.4 mM of phosphate salt,
about 0.36 mM of phosphate salt, or
about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.06% w/v of aluminum hydroxide;
about 0.05% w/v of CpG ODN; and
about 0.1 mM to about 20 mM of phosphate salt,
about 0.5 mM to about 15 mM of phosphate salt,
about 0.8 mM to about 12 mM of phosphate salt,
about 1 mM to about 10 mM of phosphate salt,
about 2 mM to about 8 mM of phosphate salt,
about 3 mM to about 7 mM of phosphate salt,
about 4 mM to about 6 mM of phosphate salt, or
about 5 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises
HA-ss-np;
about 0.6 mg/mL of aluminum as aluminum hydroxide;
about 0.5 mg/mL of CpG ODN; and
about 0.1 mM to about 20 mM of phosphate salt,
about 0.5 mM to about 15 mM of phosphate salt,
about 0.8 mM to about 12 mM of phosphate salt,
about 1 mM to about 10 mM of phosphate salt,
about 2 mM to about 8 mM of phosphate salt,
about 3 mM to about 7 mM of phosphate salt,
about 4 mM to about 6 mM of phosphate salt, or
about 5 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the phosphate salt is sodium phosphate monobasic monohydrate, and the pH of the composition is about 7.4.

In embodiments, the vaccine composition comprises a sufficiently high amount of the CpG ODN such that a binding modulator, e.g., phosphate salt, is not required for the CpG ODN to adsorb to the aluminum hydroxide and such that the HA-ss-np does not substantially adsorb to the aluminum hydroxide. In embodiments, the vaccine composition comprises HA-ss-np, aluminum hydroxide, about 0.005 to about 0.1% w/v of CpG ODN, and about 0 to about 100 mM of phosphate salt, wherein:
when the concentration of CpG ODN is about 0.005% w/v, the composition comprises at least 100 mM phosphate salt;
when the concentration of CpG ODN is about 0.01% w/v, the composition comprises at least 50 mM phosphate salt, e.g., about 50 mM, about 60 mM, about 70 mM, about 80 mM, about 90 mM, or about 100 mM phosphate salt;
when the concentration of CpG ODN is about 0.015% w/v, the composition comprises at least 20 mM phosphate salt, e.g., about 20 mM, about 30 mM, about 40 mM, about 50 mM, about 60 mM, about 70 mM, about 80 mM, about 90 mM, or about 100 mM phosphate salt;
when the concentration of CpG ODN is about 0.02% w/v, the composition comprises at least 10 mM phosphate salt, e.g., about 10 mM, about 20 mM, about 30 mM, about 40 mM, about 50 mM, about 60 mM, about 70 mM, about 80 mM, about 90 mM, or about 100 mM phosphate salt;
when the concentration of CpG ODN is about 0.025% w/v, the composition comprises at least 5 mM phosphate salt, e.g., about 5 mM, about 10 mM, about 20 mM, about 30 mM, about 40 mM, about 50 mM, about 60 mM, about 70 mM, about 80 mM, about 90 mM, or about 100 mM phosphate salt;
when the concentration of CpG ODN is about 0.03% w/v, the composition comprises at least 5 mM phosphate salt, e.g., about 5 mM, about 10 mM, about 20 mM, about 30 mM, about 40 mM, about 50 mM, about 60 mM, about 70 mM, about 80 mM, about 90 mM, or about 100 mM phosphate salt;
when the concentration of CpG ODN is about 0.035% w/v, the composition comprises at least 1 mM phosphate salt, e.g., about 1 mM, about 5 mM, about 10 mM, about 20 mM, about 30 mM, about 40 mM, about 50 mM, about 60 mM, about 70 mM, about 80 mM, about 90 mM, or about 100 mM phosphate salt;
when the concentration of CpG ODN is about 0.05% w/v, the composition comprises at least 0.5 mM phosphate salt, e.g., about 0.5 mM, about 1 mM, about 5 mM, about 10 mM, about 20 mM, about 30 mM, about 40 mM, about 50 mM, about 60 mM, about 70 mM, about 80 mM, about 90 mM, or about 100 mM phosphate salt;
when the concentration of CpG ODN is about 0.075% w/v, the composition comprises 0 to about 100 mM phosphate salt, or about 0.5 mM to about 70 mM phosphate salt, or about 1 mM to about 50 mM phosphate salt, or about 5 mM to about 20 mM phosphate salt; and
when the concentration of CpG ODN is about 0.1% w/v, the composition comprises 0 to about 100 mM phosphate salt, or about 0.5 mM to about 70 mM phosphate salt, or about 1 mM to about 50 mM phosphate salt, or about 5 mM to about 20 mM phosphate salt.

In embodiments, the vaccine composition comprises a sufficiently high amount of the CpG ODN such that a binding modulator, e.g., phosphate salt, is not required for the CpG ODN to adsorb to the aluminum hydroxide and such that the HA-ss-np does not substantially adsorb to the aluminum hydroxide. In embodiments, the vaccine composition comprises HA-ss-np, aluminum hydroxide, about 0.005 to about 0.1% w/v of CpG ODN, and about 0 to about 100 mM of phosphate salt, wherein:
when the concentration of phosphate salt is 0 mM, the composition comprises at least 0.075% w/v of CpG ODN, e.g., about 0.075% w/v or about 0.1% w/v CpG ODN;
when the concentration of phosphate salt is 0.5 mM, the composition comprises at least 0.05% w/v of CpG ODN, e.g., about 0.05% w/v, about 0.075% w/v, or about 0.1% w/v CpG ODN;
when the concentration of phosphate salt is 1 mM, the composition comprises at least 0.035% w/v of CpG ODN, e.g., about 0.035% w/v, about 0.05% w/v, about 0.075% w/v, or about 0.1% w/v CpG ODN;
when the concentration of phosphate salt is 5 mM, the composition comprises at least 0.025% w/v of CpG ODN, e.g., about 0.025% w/v, about 0.03% w/v, about 0.035% w/v, about 0.05% w/v, about 0.075% w/v, or about 0.1% w/v CpG ODN;
when the concentration of phosphate salt is 10 mM, the composition comprises at least 0.02% w/v of CpG ODN, e.g., about 0.02% w/v, about 0.025% w/v, about 0.03% w/v, about 0.035% w/v, about 0.05% w/v, about 0.075% w/v, or about 0.1% w/v CpG ODN;
when the concentration of phosphate salt is 20 mM, the composition comprises at least 0.015% w/v of CpG ODN, e.g., about 0.015% w/v, about 0.02% w/v, about 0.025% w/v, about 0.03% w/v, about 0.035% w/v, about 0.05% w/v, about 0.075% w/v, or about 0.10% w/v CpG ODN;
when the concentration of phosphate salt is 50 mM, the composition comprises at least 0.01% w/v of CpG ODN, e.g., about 0.01% w/v, about 0.015% w/v, about 0.02% w/v, about 0.025% w/v, about 0.03% w/v, about 0.035% w/v, about 0.05% w/v, about 0.075% w/v, or about 0.1% w/v CpG ODN; and
when the concentration of phosphate salt is 100 mM, the composition comprises at least 0.005% w/v of CpG ODN, e.g., about 0.005% w/v, about 0.01% w/v, about 0.015% w/v, about 0.02% w/v, about 0.025% w/v, about 0.03% w/v, about 0.035% w/v, about 0.05% w/v, about 0.075% w/v, or about 0.1% w/v CpG ODN.

### Antigen

In embodiments, the HA-ss-np is present in the vaccine composition at an amount effective to stimulate an immune response against the influenza virus. In embodiments, the HA-ss-np is present in the vaccine composition at an amount effective to confer protection against illness, including serious illness, or death from influenza. HA-ss-np is further described herein.

In embodiments, vaccine composition comprises aluminum hydroxide, CpG ODN, phosphate salt, and
about 0.001% w/v to about 0.2% w/v of HA-ss-np,
about 0.0010% w/v to about 0.10% w/v of HA-ss-np,
about 0.002% w/v to about 0.10% w/v of HA-ss-np,
about 0.002% w/v to about 0.08% w/v of HA-ss-np,
about 0.005% w/v to about 0.05% w/v of HA-ss-np,
about 0.006% w/v to about 0.04% w/v of HA-ss-np,
about 0.008% w/v to about 0.03% w/v of HA-ss-np,
about 0.008% w/v to about 0.025% w/v of HA-ss-np,
about 0.006% w/v to about 0.010% w/v of HA-ss-np,
about 0.007% w/v to about 0.009% w/v of HA-ss-np,
about 0.01% w/v to about 0.03% w/v of HA-ss-np,
about 0.010% w/v to about 0.04% w/v of HA-ss-np,
about 0.02% w/v to about 0.03% w/v of HA-ss-np,
about 0.008% w/v of HA-ss-np, or
about 0.024% w/v of HA-ss-np,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the CpG ODN is CPG 7909, the phosphate salt is sodium phosphate monobasic monohydrate, and the pH of the composition is about 7.4.

In embodiments, vaccine composition comprises about 0.005% w/v, about 0.006% w/v, about 0.007% w/v, about 0.008% w/v, about 0.009% w/v, about 0.01% w/v, about 0.012% w/v, about 0.014% w/v, about 0.016% w/v, about 0.018% w/v, about 0.02% w/v, about 0.022% w/v, about 0.024% w/v, about 0.026% w/v, about 0.028% w/v, or about 0.03% w/v of HA-ss-np, aluminum hydroxide, CpG ODN, and phosphate salt, wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the CpG ODN is CPG 7909, the phosphate salt is sodium phosphate monobasic monohydrate, and the pH of the composition is about 7.4.

In embodiments, vaccine composition comprises aluminum hydroxide, CpG ODN, phosphate salt, and
about 0.01 mg/mL to about 2 mg/mL HA-ss-np,
about 0.01 mg/mL to about 1 mg/mL of HA-ss-np,
about 0.02 mg/mL to about 1 mg/mL of HA-ss-np,
about 0.02 mg/mL to about 0.8 mg/mL of HA-ss-np,
about 0.05 mg/mL to about 0.5 mg/mL of HA-ss-np,
about 0.06 mg/mL to about 0.4 mg/mL of HA-ss-np,
about 0.08 mg/mL to about 0.3 mg/mL of HA-ss-np,
about 0.08 mg/mL to about 0.25 mg/mL of HA-ss-np,
about 0.06 mg/mL to about 0.1 mg/mL of HA-ss-np,
about 0.07 mg/mL to about 0.09 mg/mL of HA-ss-np,
about 0.1 mg/mL to about 0.3 mg/mL of HA-ss-np,
about 0.1 mg/mL to about 0.4 mg/mL of HA-ss-np,
about 0.2 mg/mL to about 0.3 mg/mL of HA-ss-np,
about 0.08 mg/mL of HA-ss-np, or
about 0.24 mg/mL of HA-ss-np,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the CpG ODN is CPG 7909, the phosphate salt is sodium phosphate monobasic monohydrate, and the pH of the composition is about 7.4.

In embodiments, vaccine composition comprises about 0.05 mg/mL, about 0.06 mg/mL, about 0.07 mg/mL, about 0.08 mg/mL, about 0.09 mg/mL, about 0.1 mg/mL, about 0.12 mg/mL, about 0.14 mg/mL, about 0.16 mg/mL, about 0.18 mg/mL, about 0.2 mg/mL, about 0.22 mg/mL, about 0.24 mg/mL, about 0.26 mg/mL, about 0.28 mg/mL, or about 0.3 mg/mL of HA-ss-np, aluminum hydroxide, CpG ODN, and phosphate salt, wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises A1-ss-np and A2-ss-np, the CpG ODN is CPG 7909, the CpG ODN is CPG 7909, the phosphate salt is sodium phosphate monobasic monohydrate, and the pH of the composition is about 7.4.

In embodiments, the HA-ss-np comprises an HA-ss from a Group 1 influenza virus, an HA-ss from a Group 2 influenza virus, a Type B influenza virus, a Type C influenza virus, or a combination thereof. In embodiments, the HA-ss-np comprises an HA-ss from a Group 1 influenza virus (A1-ss-np). In embodiments, the Group 1 influenza is subgroup H1. In embodiments, the HA-ss-np comprises an HA-ss from a Group 2 influenza virus (A2-ss-np). In embodiments, the Group 2 influenza is subgroup H10. In embodiments, the vaccine composition comprises an A1-ss-np and A2-ss-np. In embodiments, the vaccine composition comprises an HA-ss-np from a Type A influenza virus and an HA-ss-np from a Type B influenza virus. Types and Groups of influenza viruses are further described herein. In embodiments, the HA-ss-np comprises an A1-ss-np and an A2-ss-np at a ratio of about 1:1. In embodiments, the A1-ss-np and A2-ss-np are at a ratio of about 5:1, about 4.5:1, about 4:1, about 3.5:1, about 3:1, about 2.5:1, about 2:1, about 1.7:1, about 1.5:1, about 1.2:1, about 1:1, about 1:1.2, about 1:1.5, about 1:1.7, about 1:2, about 1:2.5, about 1:3, about 1:3.5, about 1:4, about 1:4.5, or about 1:5.

In embodiments, vaccine composition comprises aluminum hydroxide, CpG ODN, phosphate salt, about 0.0005% w/v to about 0.1% w/v of A1-ss-np, and about 0.0005% w/v to about 0.10% w/v of A2-ss-np.

In embodiments, vaccine composition comprises aluminum hydroxide, CpG ODN, phosphate salt, and
about 0.0005% w/v to about 0.1% w/v of each of A1-ss-np and A2-ss-np,
about 0.0005% w/v to about 0.05% w/v of each of A1-ss-np and A2-ss-np,
about 0.001% w/v to about 0.05% w/v of each of A1-ss-np and A2-ss-np,
about 0.001% w/v to about 0.04% w/v of each of A1-ss-np and A2-ss-np,
about 0.0025% w/v to about 0.025% w/v of each of A1-ss-np and A2-ss-np,
about 0.003% w/v to about 0.02% w/v of each of A1-ss-np and A2-ss-np,
about 0.004% w/v to about 0.015% w/v of each of A1-ss-np and A2-ss-np,
about 0.004% w/v to about 0.012% w/v of each of A1-ss-np and A2-ss-np,
about 0.003% w/v to about 0.005% w/v of each of A1-ss-np and A2-ss-np,
about 0.0035% w/v to about 0.0045% w/v of each of A1-ss-np and A2-ss-np,
about 0.005% w/v to about 0.015% w/v of each of A1-ss-np and A2-ss-np,
about 0.005% w/v to about 0.02% w/v of each of A1-ss-np and A2-ss-np,
about 0.01% w/v to about 0.015% w/v of each of A1-ss-np and A2-ss-np,
about 0.004% w/v of each of A1-ss-np and A2-ss-np, or
about 0.012% w/v of each of A1-ss-np and A2-ss-np,
   wherein the A1-ss-np and A2-ss-np are not substantially adsorbed to the aluminum hydroxide, and wherein at least a portion of the CpG ODN is adsorbed to the aluminum hydroxide in the composition. In embodiments, the CpG ODN is CPG 7909. In embodiments, the phosphate salt is sodium phosphate monobasic monohydrate. In embodiments, the pH of the composition is about 7.4. In embodiments, the CpG ODN is CPG 7909, the phosphate salt is sodium phosphate monobasic monohydrate, and the pH of the composition is about 7.4.

In embodiments, the vaccine composition of the invention comprises
about 0.001% w/v to about 0.2% w/v of HA-ss-np,
about 0.01% w/v to about 1% w/v of aluminum hydroxide,
about 0.01% w/v to about 0.5% w/v of CpG ODN, and
about 0.2 mM to about 4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.002% w/v to about 0.10% w/v of HA-ss-np,
about 0.02% w/v to about 0.15% w/v of aluminum hydroxide,
about 0.01% w/v to about 0.12% w/v of CpG ODN, and
about 0.2 mM to about 1 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.005% w/v to about 0.05% w/v of HA-ss-np,
about 0.04% w/v to about 0.08% w/v of aluminum hydroxide,
about 0.08% w/v to about 0.15% w/v of CpG ODN,
and about 0.3 mM to about 0.7 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.008% w/v to about 0.03% w/v of HA-ss-np,
about 0.05% w/v to about 0.07% w/v of aluminum hydroxide,
about 0.06% w/v to about 0.12% w/v of CpG ODN,
and about 0.3 mM to about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition of the invention comprises
about 0.001% w/v to about 0.2% w/v of HA-ss-np,
about 0.01% w/v to about 1% w/v of aluminum hydroxide,
about 0.01% w/v to about 0.5% w/v of CpG ODN, and
about 0.2 mM to about 20 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.002% w/v to about 0.10% w/v of HA-ss-np,
about 0.02% w/v to about 0.15% w/v of aluminum hydroxide,
about 0.01% w/v to about 0.2% w/v of CpG ODN, and
about 0.5 mM to about 15 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.005% w/v to about 0.05% w/v of HA-ss-np,
about 0.04% w/v to about 0.08% w/v of aluminum hydroxide,
about 0.02% w/v to about 0.1% w/v of CpG ODN,
and about 1 mM to about 10 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.008% w/v to about 0.03% w/v of HA-ss-np,
about 0.05% w/v to about 0.07% w/v of aluminum hydroxide,
about 0.02% w/v to about 0.08% w/v of CpG ODN,
and about 2 mM to about 8 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition of the invention comprises
about 0.01 mg/mL to about 2 mg/mL of HA-ss-np,
about 0.1 mg/mL to about 1 mg/mL of aluminum as aluminum hydroxide,
about 0.1 mg/mL to about 5 mg/mL of CpG ODN, and
about 0.2 mM to about 4 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the HA-ss-np comprises A1-ss-np and A2-ss-np, the CpG ODN is CPG 7909, and the phosphate salt is sodium phosphate monobasic monohydrate. In embodiments, the pH of the composition is about 7.4.

In embodiments, the vaccine composition comprises
about 0.02 mg/mL to about 1 mg/mL of HA-ss-np,
about 0.2 mg/mL to about 1.5 mg/mL of aluminum as aluminum hydroxide,
about 0.1 mg/mL to about 1.2 mg/mL of CpG ODN, and
about 0.2 mM to about 1 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.05 mg/mL to about 0.5 mg/mL of HA-ss-np,
about 0.4 mg/mL to about 0.8 mg/mL of aluminum as aluminum hydroxide,
about 0.8 mg/mL to about 1.5 mg/mL of CpG ODN, and
about 0.3 mM to about 0.7 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.08 mg/mL to about 0.3 mg/mL of HA-ss-np,
about 0.5 mg/mL to about 0.7 mg/mL of aluminum as aluminum hydroxide,
about 0.6 mg/mL to about 1.2 mg/mL of CpG ODN, and
about 0.3 mM to about 0.4 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition of the invention comprises
about 0.01 mg/mL to about 2 mg/mL of HA-ss-np,
about 0.1 mg/mL to about 1 mg/mL of aluminum as aluminum hydroxide,
about 0.1 mg/mL to about 5 mg/mL of CpG ODN, and
about 0.2 mM to about 20 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the HA-ss-np comprises A1-ss-np and A2-ss-np, the CpG ODN is CPG 7909, and the phosphate salt is sodium phosphate monobasic monohydrate. In embodiments, the pH of the composition is about 7.4.

In embodiments, the vaccine composition comprises
about 0.02 mg/mL to about 1 mg/mL of HA-ss-np,
about 0.2 mg/mL to about 1.5 mg/mL of aluminum as aluminum hydroxide,
about 0.1 mg/mL to about 2 mg/mL of CpG ODN, and
about 0.5 mM to about 15 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.05 mg/mL to about 0.5 mg/mL of HA-ss-np,
about 0.4 mg/mL to about 0.8 mg/mL of aluminum as aluminum hydroxide,
about 0.2 mg/mL to about 1 mg/mL of CpG ODN, and
about 1 mM to about 10 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.08 mg/mL to about 0.3 mg/mL of HA-ss-np,
about 0.5 mg/mL to about 0.7 mg/mL of aluminum as aluminum hydroxide,
about 0.2 mg/mL to about 0.8 mg/mL of CpG ODN, and
about 2 mM to about 8 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.001% w/v to about 0.2% w/v of HA-ss-np,
about 0.002% w/v to about 0.1% w/v of HA-ss-np,
about 0.005% w/v to about 0.05% w/v of HA-ss-np, or
about 0.008% w/v to about 0.03% w/v of HA-ss-np;
about 0.06 w/v of aluminum hydroxide;
about 0.10% w/v of CpG ODN; and
about 0.36 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.01 mg/mL to about 2 mg/mL of HA-ss-np,
about 0.02 mg/mL to about 1 mg/mL of HA-ss-np,
about 0.05 mg/mL to about 0.5 mg/mL of HA-ss-np,
about 0.06 mg/mL to about 0.4 mg/mL of HA-ss-np, or
about 0.08 mg/mL to about 0.3 mg/mL of HA-ss-np;
about 0.6 mg/mL of aluminum as aluminum hydroxide;
about 1 mg/mL of CpG ODN; and
about 0.36 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.001% w/v to about 0.2% w/v of HA-ss-np,
about 0.002% w/v to about 0.1% w/v of HA-ss-np,
about 0.005% w/v to about 0.05% w/v of HA-ss-np, or
about 0.008% w/v to about 0.03% w/v of HA-ss-np;
about 0.06 w/v of aluminum hydroxide;
about 0.05% w/v of CpG ODN; and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.01 mg/mL to about 2 mg/mL of HA-ss-np,
about 0.02 mg/mL to about 1 mg/mL of HA-ss-np,
about 0.05 mg/mL to about 0.5 mg/mL of HA-ss-np,
about 0.06 mg/mL to about 0.4 mg/mL of HA-ss-np, or
about 0.08 mg/mL to about 0.3 mg/mL of HA-ss-np;
about 0.6 mg/mL of aluminum as aluminum hydroxide;
about 0.5 mg/mL of CpG ODN; and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.008% w/v HA-ss-np;
about 0.01% w/v to about 1% w/v of aluminum hydroxide,
about 0.02% w/v to about 0.15% w/v of aluminum hydroxide, or
about 0.04% w/v to about 0.08% w/v of aluminum hydroxide;
about 0.10% w/v of CpG ODN; and
about 0.36 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.024% w/v HA-ss-np;
about 0.010% w/v to about 1% w/v of aluminum hydroxide,
about 0.02% w/v to about 0.15% w/v of aluminum hydroxide,
about 0.04% w/v to about 0.08% w/v of aluminum hydroxide, or
about 0.06% w/v of aluminum hydroxide;
about 0.10% w/v of CpG ODN; and
about 0.36 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.008% w/v HA-ss-np;
about 0.010% w/v to about 1% w/v of aluminum hydroxide,
about 0.02% w/v to about 0.15% w/v of aluminum hydroxide, or
about 0.04% w/v to about 0.08% w/v of aluminum hydroxide;
about 0.05% w/v of CpG ODN; and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.024% w/v HA-ss-np;
about 0.01% w/v to about 1% w/v of aluminum hydroxide,
about 0.02% w/v to about 0.15% w/v of aluminum hydroxide,
about 0.04% w/v to about 0.08% w/v of aluminum hydroxide, or
about 0.06% w/v of aluminum hydroxide;
about 0.05% w/v of CpG ODN; and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.08 mg/mL HA-ss-np;
about 0.1 mg/mL to about 10 mg/mL of aluminum as aluminum hydroxide,
about 0.2 mg/mL to about 1.5 mg/mL of aluminum as aluminum hydroxide,
about 0.4 mg/mL to about 0.8 mg/mL of aluminum as aluminum hydroxide, or
about 0.6 mg/mL of aluminum as aluminum hydroxide;
about 1 mg/mL of CpG ODN; and
about 0.36 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.24 mg/mL HA-ss-np;
about 0.1 mg/mL to about 10 mg/mL of aluminum as aluminum hydroxide,
about 0.2 mg/mL to about 1.5 mg/mL of aluminum as aluminum hydroxide,
about 0.4 mg/mL to about 0.8 mg/mL of aluminum as aluminum hydroxide, or
about 0.6 mg/mL of aluminum as aluminum hydroxide;
about 1 mg/mL of CpG ODN; and
and about 0.36 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.08 mg/mL HA-ss-np;
about 0.1 mg/mL to about 10 mg/mL of aluminum as aluminum hydroxide,
about 0.2 mg/mL to about 1.5 mg/mL of aluminum as aluminum hydroxide,
about 0.4 mg/mL to about 0.8 mg/mL of aluminum as aluminum hydroxide, or
about 0.6 mg/mL of aluminum as aluminum hydroxide;
about 0.5 mg/mL of CpG ODN; and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.24 mg/mL HA-ss-np;
about 0.1 mg/mL to about 10 mg/mL of aluminum as aluminum hydroxide,
about 0.2 mg/mL to about 1.5 mg/mL of aluminum as aluminum hydroxide,
about 0.4 mg/mL to about 0.8 mg/mL of aluminum as aluminum hydroxide, or
about 0.6 mg/mL of aluminum as aluminum hydroxide;
about 0.5 mg/mL of CpG ODN; and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.008% w/v of HA-ss-np;
about 0.06 w/v of aluminum hydroxide;
about 0.01% w/v to about 0.5% w/v of CpG ODN,
about 0.01% w/v to about 0.12% w/v of CpG ODN,
about 0.08% w/v to about 0.15% w/v of CpG ODN,
about 0.06% w/v to about 0.12% w/v of CpG ODN,
about 0.05% w/v to about 0.1% w/v of CpG ODN,
about 0.02% w/v to about 0.08% w/v of CpG ODN,
about 0.05% w/v of CpG ODN, or
about 0.10% w/v of CpG ODN; and
about 0.36 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.024% w/v of HA-ss-np;
about 0.06 w/v of aluminum hydroxide;
about 0.01% w/v to about 0.5% w/v of CpG ODN,
about 0.01% w/v to about 0.12% w/v of CpG ODN,
about 0.08% w/v to about 0.15% w/v of CpG ODN,
about 0.06% w/v to about 0.12% w/v of CpG ODN,
about 0.05% w/v to about 0.1% w/v of CpG ODN,
about 0.02% w/v to about 0.08% w/v of CpG ODN,
about 0.05% w/v of CpG ODN, or
about 0.1% w/v of CpG ODN; and
about 0.36 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.08 mg/mL HA-ss-np;
about 0.6 mg/mL of aluminum as aluminum hydroxide;
about 0.1 mg/mL to about 5 mg/mL of CpG ODN,
about 0.1 mg/mL to about 1.2 mg/mL of CpG ODN,
about 0.8 mg/mL to about 1.5 mg/mL of CpG ODN,
about 0.6 mg/mL to about 1.2 mg/mL of CpG ODN,
about 0.5 mg/mL to about 1 mg/mL of CpG ODN,
about 0.2 mg/mL to about 0.8 mg/mL of CpG ODN,
about 0.5 mg/mL of CpG ODN, or
about 1 mg/mL of CpG ODN; and
about 0.36 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.24 mg/mL HA-ss-np;
about 0.6 mg/mL of aluminum as aluminum hydroxide;
about 0.1 mg/mL to about 5 mg/mL of CpG ODN,
about 0.1 mg/mL to about 1.2 mg/mL of CpG ODN,
about 0.8 mg/mL to about 1.5 mg/mL of CpG ODN,
about 0.6 mg/mL to about 1.2 mg/mL of CpG ODN,
about 0.5 mg/mL to about 1 mg/mL of CpG ODN,
about 0.2 mg/mL to about 0.8 mg/mL of CpG ODN,
about 0.5 mg/mL of CpG ODN, or
about 1 mg/mL of CpG ODN; and
about 0.36 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.008% w/v of HA-ss-np;
about 0.06 w/v of aluminum hydroxide;
about 0.01% w/v to about 0.5% w/v of CpG ODN,
about 0.010% w/v to about 0.12% w/v of CpG ODN,
about 0.08% w/v to about 0.15% w/v of CpG ODN,
about 0.06% w/v to about 0.12% w/v of CpG ODN,
about 0.05% w/v to about 0.1% w/v of CpG ODN,
about 0.02% w/v to about 0.08% w/v of CpG ODN,
about 0.05% w/v of CpG ODN, or
about 0.10% w/v of CpG ODN; and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.024% w/v of HA-ss-np;
about 0.06 w/v of aluminum hydroxide;
about 0.01% w/v to about 0.5% w/v of CpG ODN,
about 0.010% w/v to about 0.12% w/v of CpG ODN,
about 0.08% w/v to about 0.15% w/v of CpG ODN,
about 0.06% w/v to about 0.12% w/v of CpG ODN,
about 0.05% w/v to about 0.1% w/v of CpG ODN,
about 0.02% w/v to about 0.08% w/v of CpG ODN,
about 0.05% w/v of CpG ODN, or
about 0.10% w/v of CpG ODN; and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.08 mg/mL HA-ss-np;
about 0.6 mg/mL of aluminum as aluminum hydroxide;
about 0.1 mg/mL to about 5 mg/mL of CpG ODN,
about 0.1 mg/mL to about 1.2 mg/mL of CpG ODN,
about 0.8 mg/mL to about 1.5 mg/mL of CpG ODN,
about 0.6 mg/mL to about 1.2 mg/mL of CpG ODN,
about 0.5 mg/mL to about 1 mg/mL of CpG ODN,
about 0.2 mg/mL to about 0.8 mg/mL of CpG ODN,
about 0.5 mg/mL of CpG ODN, or
about 1 mg/mL of CpG ODN; and
about 5 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.24 mg/mL HA-ss-np;
about 0.6 mg/mL of aluminum as aluminum hydroxide;
about 0.1 mg/mL to about 5 mg/mL of CpG ODN,
about 0.1 mg/mL to about 1.2 mg/mL of CpG ODN,
about 0.8 mg/mL to about 1.5 mg/mL of CpG ODN,
about 0.6 mg/mL to about 1.2 mg/mL of CpG ODN,
about 0.5 mg/mL to about 1 mg/mL of CpG ODN,
about 0.2 mg/mL to about 0.8 mg/mL of CpG ODN,
about 0.5 mg/mL of CpG ODN, or
about 1 mg/mL of CpG ODN; and
about 5 mM phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.008% w/v of HA-ss-np;
about 0.06% w/v of aluminum hydroxide;
about 0.10% w/v of CpG ODN; and
about 0.1 mM to about 50 mM of phosphate salt,
about 0.5 mM to about 20 mM of phosphate salt,
about 1 mM to about 15 mM of phosphate salt,
about 2 mM to about 10 mM of phosphate salt,
about 0.2 mM to about 4 mM of phosphate salt,
about 0.2 mM to about 1 mM of phosphate salt,
about 0.3 mM to about 0.8 mM of phosphate salt,
about 0.3 mM to about 0.5 mM of phosphate salt,
about 0.2 mM to about 0.6 mM of phosphate salt,
about 0.35 mM to about 0.7 mM of phosphate salt,
about 0.4 mM to about 0.7 mM of phosphate salt,
about 0.3 mM to about 0.4 mM of phosphate salt,
about 0.35 mM to about 0.4 mM of phosphate salt,
about 5 mM of phosphate salt,
about 0.36 mM of phosphate salt, or
about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.024% w/v of HA-ss-np;
about 0.06% w/v of aluminum hydroxide;
about 0.10% w/v of CpG ODN; and
about 0.1 mM to about 50 mM of phosphate salt,
about 0.5 mM to about 20 mM of phosphate salt,
about 1 mM to about 15 mM of phosphate salt,
about 2 mM to about 10 mM of phosphate salt,
about 0.2 mM to about 4 mM of phosphate salt,
about 0.2 mM to about 1 mM of phosphate salt,
about 0.3 mM to about 0.8 mM of phosphate salt,
about 0.3 mM to about 0.5 mM of phosphate salt,
about 0.2 mM to about 0.6 mM of phosphate salt,
about 0.35 mM to about 0.7 mM of phosphate salt,
about 0.4 mM to about 0.7 mM of phosphate salt,
about 0.3 mM to about 0.4 mM of phosphate salt,
about 0.35 mM to about 0.4 mM of phosphate salt,
about 5 mM of phosphate salt,
about 0.36 mM of phosphate salt, or
about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.08 mg/mL of HA-ss-np;
about 0.6 mg/mL of aluminum as aluminum hydroxide;
about 1 mg/mL of CpG ODN; and
about 0.1 mM to about 50 mM of phosphate salt,
about 0.5 mM to about 20 mM of phosphate salt,
about 1 mM to about 15 mM of phosphate salt,
about 2 mM to about 10 mM of phosphate salt,
about 0.2 mM to about 4 mM of phosphate salt,
about 0.2 mM to about 1 mM of phosphate salt,
about 0.3 mM to about 0.8 mM of phosphate salt,
about 0.3 mM to about 0.5 mM of phosphate salt,
about 0.2 mM to about 0.6 mM of phosphate salt,
about 0.35 mM to about 0.7 mM of phosphate salt,
about 0.4 mM to about 0.7 mM of phosphate salt,
about 0.3 mM to about 0.4 mM of phosphate salt,
about 0.35 mM to about 0.4 mM of phosphate salt,
about 5 mM of phosphate salt,
about 0.36 mM of phosphate salt, or
about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.24 mg/mL of HA-ss-np;
about 0.6 mg/mL of aluminum as aluminum hydroxide;
about 1 mg/mL of CpG ODN; and
about 0.1 mM to about 50 mM of phosphate salt,
about 0.5 mM to about 20 mM of phosphate salt,
about 1 mM to about 15 mM of phosphate salt,
about 2 mM to about 10 mM of phosphate salt,
about 0.2 mM to about 4 mM of phosphate salt,
about 0.2 mM to about 1 mM of phosphate salt,
about 0.3 mM to about 0.8 mM of phosphate salt,
about 0.3 mM to about 0.5 mM of phosphate salt,
about 0.2 mM to about 0.6 mM of phosphate salt,
about 0.35 mM to about 0.7 mM of phosphate salt,
about 0.4 mM to about 0.7 mM of phosphate salt,
about 0.3 mM to about 0.4 mM of phosphate salt,
about 0.35 mM to about 0.4 mM of phosphate salt,
about 5 mM of phosphate salt,
about 0.36 mM of phosphate salt, or
about 0.4 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.008% w/v of HA-ss-np,
about 0.06% w/v of aluminum hydroxide,
about 0.1% w/v of CpG ODN, and
about 0.36 mM of the phosphate salt,
   wherein pH of the composition is about 6.5 to about 8, about 6.8 to about 8, about 6.7 to about 7.7, about 7 to about 7.9, about 7.2 to about 7.8, about 7.3 to about 7.6, or about 7.4 to about 7.5, and wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.024% w/v of HA-ss-np,
about 0.06% w/v of aluminum hydroxide,
about 0.1% w/v of CpG ODN, and
about 0.36 mM of the phosphate salt,
   wherein pH of the composition is about 6.5 to about 8, about 6.8 to about 8, about 6.7 to about 7.7, about 7 to about 7.9, about 7.2 to about 7.8, about 7.3 to about 7.6, or about 7.4 to about 7.5, and wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.008% w/v of HA-ss-np,
about 0.06% w/v of aluminum hydroxide,
about 0.1% w/v of CpG ODN, and
about 0.36 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.024% w/v of HA-ss-np,
about 0.06% w/v of aluminum hydroxide,
about 0.1% w/v of CpG ODN, and
about 0.36 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.08 mg/mL of HA-ss-np,
about 0.6 mg/mL of aluminum as aluminum hydroxide,
about 1 mg/mL of CpG ODN, and
about 0.36 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.24 mg/mL of HA-ss-np,
about 0.6 mg/mL of aluminum as aluminum hydroxide,
about 1 mg/mL of CpG ODN, and
about 0.36 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.008% w/v of HA-ss-np,
about 0.06% w/v of aluminum hydroxide,
about 0.05% w/v of CpG ODN, and
about 5 mM of the phosphate salt,
   wherein pH of the composition is about 6.5 to about 8, about 6.8 to about 8, about 6.7 to about 7.7, about 7 to about 7.9, about 7.2 to about 7.8, about 7.3 to about 7.6, or about 7.4 to about 7.5, and wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.024% w/v of HA-ss-np,
about 0.06% w/v of aluminum hydroxide,
about 0.05% w/v of CpG ODN, and
about 5 mM of the phosphate salt,
   wherein pH of the composition is about 6.5 to about 8, about 6.8 to about 8, about 6.7 to about 7.7, about 7 to about 7.9, about 7.2 to about 7.8, about 7.3 to about 7.6, or about 7.4 to about 7.5, and wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.008% w/v of HA-ss-np,
about 0.06% w/v of aluminum hydroxide,
about 0.05% w/v of CpG ODN, and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.024% w/v of HA-ss-np,
about 0.06% w/v of aluminum hydroxide,
about 0.05% w/v of CpG ODN, and
about 5 mM of the phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.08 mg/mL of HA-ss-np,
about 0.6 mg/mL of aluminum as aluminum hydroxide,
about 0.5 mg/mL of CpG ODN, and
about 5 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

In embodiments, the vaccine composition comprises
about 0.24 mg/mL of HA-ss-np,
about 0.6 mg/mL of aluminum as aluminum hydroxide,
about 0.5 mg/mL of CpG ODN, and
about 5 mM of phosphate salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the HA-ss-np comprises an A1-ss-np and an A2-ss-np, the CpG ODN is CPG 7909, the phosphate salt is sodium phosphate monobasic monohydrate, and the pH of the composition is about 7.4.

### Additional Components

In embodiments, the vaccine compositions provided herein further comprise an additional adjuvant. Exemplary adjuvants are provided herein. In embodiments, the vaccine composition comprises HA-ss-np, aluminum hydroxide, CpG ODN, a phosphate salt, and an additional adjuvant selected from QS-21, monophosphoryl lipid A (MPLA), glucopyranosyl lipid (GLA), and combinations thereof, wherein the HA-ss-np is not substantially adsorbed to the aluminum hydroxide, and wherein at least a portion of the CpG ODN is adsorbed to the aluminum hydroxide. In embodiments, the vaccine composition comprises HA-ss-np, aluminum hydroxide, CpG ODN, a phosphate salt, and QS-21, wherein the HA-ss-np is not substantially adsorbed to the aluminum hydroxide, and wherein at least a portion of the CpG ODN is adsorbed to the aluminum hydroxide. In embodiments, at least a portion of the additional adjuvant is adsorbed to the aluminum hydroxide.

In embodiments, the vaccine compositions provided herein further comprises a pharmaceutically acceptable excipient. In embodiments, the vaccine composition further comprises a surfactant, a salt, a buffering agent, a cryoprotectant, or a combination thereof.

In embodiments, the surfactant is an ionic surfactant. In embodiments, the surfactant is a non-ionic surfactant. In embodiments, the surfactant is polysorbate 80, Triton X-100, or polysorbate 20. In embodiments, the surfactant comprises polysorbate 80.

In embodiments, the vaccine composition comprises HA-ss-np; aluminum hydroxide; CpG ODN; a phosphate salt; a salt, a buffering agent, a cryoprotectant, or a combination thereof, and
about 0.005% w/v to about 0.05% w/v of a polysorbate,
about 0.01% w/v to about 0.02% w/v of a polysorbate,
about 0.014% w/v to about 0.015% w/v of a polysorbate,
about 0.014% w/v of a polysorbate, or
about 0.015% w/v of a polysorbate,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the polysorbate is polysorbate 80. In embodiments, the salt is NaCl. In embodiments, the buffering agent is Tris-HCl. In embodiments, the cryoprotectant is sucrose. In embodiments, the HA-ss-np comprises an A1-ss-np and an A2-ss-np, the CpG ODN is CPG 7909, the phosphate salt is sodium phosphate monobasic monohydrate, the pH of the composition is about 7.4, and polysorbate is polysorbate 80, the salt is NaCl, the buffering agent is Tris-HCl, and the cryoprotectant is sucrose.

In embodiments, the salt is a non-phosphate salt. In embodiments, the salt comprises a chloride ion. In embodiments, the salt comprises an acetate ion. In embodiments, the salt is a sodium salt. In embodiments, the salt is a potassium salt. In embodiments, the salt comprises sodium chloride (NaCl), potassium chloride (KCl), calcium chloride (CaCl₂)), sodium acetate, or combination thereof. In embodiments, the salt comprises NaCl.

In embodiments, the vaccine composition comprises HA-ss-np; aluminum hydroxide; CpG ODN; a phosphate salt; a surfactant, a buffering agent, a cryoprotectant, or a combination thereof; and
about 10 mM to about 500 mM salt,
about 50 mM to about 200 mM salt,
about 75 mM to about 175 mM salt, or
about 150 mM salt,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the polysorbate is polysorbate 80. In embodiments, the salt is NaCl. In embodiments, the buffering agent is Tris-HCl. In embodiments, the cryoprotectant is sucrose. In embodiments, the HA-ss-np comprises an A1-ss-np and an A2-ss-np, the CpG ODN is CPG 7909, the phosphate salt is sodium phosphate monobasic monohydrate, the pH of the composition is about 7.4, and polysorbate is polysorbate 80, the salt is NaCl, the buffering agent is Tris-HCl, and the cryoprotectant is sucrose.

In embodiments, the buffering agent is capable of maintaining pH of the vaccine composition at about 6.5 to about 8, about 6.8 to about 8, about 6.7 to about 7.7, about 7 to about 7.9, about 7.2 to about 7.8, about 7.3 to about 7.6, or about 7.4 to about 7.5. In embodiments, the buffering agent comprises Tris-HCl, PIPES, MOPS, HEPES, TEA, or combination thereof. In embodiments, the buffering agent comprises Tris-HCl.

In embodiments, the vaccine composition comprises HA-ss-np; aluminum hydroxide; CpG ODN; a phosphate salt; one or more of a surfactant, a salt, a cryoprotectant, or a combination thereof; and
about 1 mM to about 10 mM of a buffering agent,
about 2 mM to about 8 mM of a buffering agent,
about 3 mM to about 6 mM of a buffering agent, or
about 5 mM of a buffering agent,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the polysorbate is polysorbate 80. In embodiments, the salt is NaCl. In embodiments, the buffering agent is Tris-HCl. In embodiments, the cryoprotectant is sucrose. In embodiments, the HA-ss-np comprises an A1-ss-np and an A2-ss-np, the CpG ODN is CPG 7909, the phosphate salt is sodium phosphate monobasic monohydrate, the pH of the composition is about 7.4, and polysorbate is polysorbate 80, the salt is NaCl, the buffering agent is Tris-HCl, and the cryoprotectant comprises sucrose.

In embodiments, the cryoprotectant prevents aggregation of the HA-ss-np after freeze-thawing the vaccine composition. In embodiments, the cryoprotectant maintains stability of the vaccine composition when the composition is stored at about −80° C. to about 10° C., or about −70° C. to about 4° C., or about −60° C. to about 0° C., or about −50° C. to about −10° C., or about −40° C. to about −15° C., or about −30° C. to about −20° C., or about −20° C. to about 0° C. In embodiments, the cryoprotectant comprises a sugar. In embodiments, the cryoprotectant comprises a disaccharide such as lactose, maltose, sucrose, or trehalose, a polyhydroxy hydrocarbon such as dulcitol, glycerol, mannitol, or sorbitol, or a combination thereof. In embodiments, the cryoprotectant is sucrose.

In embodiments, the vaccine composition comprises HA-ss-np; aluminum hydroxide; CpG ODN; a phosphate salt; one or more of a surfactant, a salt, a buffering agent, or a combination thereof; and
about 0.5% w/v to about 25% w/v of a cryoprotectant,
about 1% w/v to about 25% w/v of a cryoprotectant,
about 1% w/v to about 20% w/v of a cryoprotectant,
about 1% w/v to about 15% w/v of a cryoprotectant,
about 0.5% w/v to about 10% w/v of a cryoprotectant,
about 1% w/v to about 8% w/v of a cryoprotectant, or
about 5% w/v of a cryoprotectant,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the phosphate salt is sodium phosphate monobasic monohydrate. In embodiments, the pH of the composition is about 7.4. In embodiments, the polysorbate is polysorbate 80. In embodiments, the salt is NaCl. In embodiments, the buffering agent is Tris-HCl. In embodiments, the cryoprotectant is sucrose. In embodiments, the HA-ss-np comprises an A1-ss-np and an A2-ss-np, the CpG ODN is CPG 7909, the phosphate salt is sodium phosphate monobasic monohydrate, the pH of the composition is about 7.4, and polysorbate is polysorbate 80, the salt is NaCl, the buffering agent is Tris-HCl, and the cryoprotectant comprises sucrose.

In embodiments, the vaccine composition comprises
about 0.001% w/v to about 0.2% w/v of HA-ss-np,
about 0.01% w/v to about 1% w/v of aluminum hydroxide,
about 0.01% w/v to about 0.5% w/v of CpG ODN,
about 0.2 mM to about 4 mM of sodium phosphate monobasic monohydrate,
about 0.005% w/v to about 0.05% w/v of polysorbate 80,
about 10 mM to about 500 mM NaCl,
about 1 mM to about 10 mM of Tris-HCl, and
about 1% w/v to about 25% w/v of sucrose,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the pH of the composition is about 7.4. In embodiments, the HA-ss-np comprises an A1-ss-np and an A2-ss-np, the CpG ODN is CPG 7909, and the pH of the composition is about 7.4.

In embodiments, the vaccine composition comprises
about 0.008% w/v to about 0.03% w/v of HA-ss-np,
about 0.05% w/v to about 0.07% w/v of aluminum hydroxide,
about 0.06% w/v to about 0.12% w/v of CpG ODN,
about 0.3 mM to about 0.4 mM of sodium phosphate monobasic monohydrate,
about 0.010% w/v to about 0.02% w/v of polysorbate 80,
about 50 mM to about 200 mM NaCl,
about 2 mM to about 8 mM Tris-HCl, and
about 1% w/v to about 15% w/v sucrose,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the pH of the composition is about 7.4. In embodiments, the HA-ss-np comprises an A1-ss-np and an A2-ss-np, the CpG ODN is CPG 7909, and the pH of the composition is about 7.4.

In embodiments, the vaccine composition comprises
about 0.001% w/v to about 0.2% w/v of HA-ss-np,
about 0.01% w/v to about 1% w/v of aluminum hydroxide,
about 0.01% w/v to about 0.5% w/v of CpG ODN,
about 0.2 mM to about 20 mM of sodium phosphate monobasic monohydrate,
about 0.005% w/v to about 0.05% w/v of polysorbate 80,
about 10 mM to about 500 mM NaCl,
about 1 mM to about 10 mM of Tris-HCl, and
about 1% w/v to about 25% w/v of sucrose,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the pH of the composition is about 7.4. In embodiments, the HA-ss-np comprises an A1-ss-np and an A2-ss-np, the CpG ODN is CPG 7909, and the pH of the composition is about 7.4.

In embodiments, the vaccine composition comprises
about 0.008% w/v to about 0.03% w/v of HA-ss-np,
about 0.05% w/v to about 0.07% w/v of aluminum hydroxide,
about 0.02% w/v to about 0.08% w/v of CpG ODN,
about 2 mM to about 8 mM of sodium phosphate monobasic monohydrate,
about 0.010% w/v to about 0.02% w/v of polysorbate 80,
about 50 mM to about 200 mM NaCl,
about 2 mM to about 8 mM Tris-HCl, and
about 1% w/v to about 15% w/v sucrose,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the pH of the composition is about 7.4. In embodiments, the HA-ss-np comprises an A1-ss-np and an A2-ss-np, the CpG ODN is CPG 7909, and the pH of the composition is about 7.4.

In embodiments, the vaccine composition comprises
about 0.008% w/v or about 0.024% w/v of HA-ss-np,
about 0.06% w/v of aluminum hydroxide,
about 0.1% w/v of CpG ODN,
about 0.36 mM of sodium phosphate monobasic monohydrate,
about 0.015% w/v of a polysorbate 80,
about 150 mM NaCl,
about 5 mM Tris-HCl, and
about 5% w/v of sucrose,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the pH of the composition is about 7.4. In embodiments, the HA-ss-np comprises an A1-ss-np and an A2-ss-np, the CpG ODN is CPG 7909, and the pH of the composition is about 7.4.

In embodiments, the vaccine composition comprises
about 0.08 mg/mL or about 0.24 mg/mL of HA-ss-np,
about 0.6 mg/mL of aluminum hydroxide,
about 1 mg/mL of CpG ODN, and
about 0.35 mM sodium phosphate monobasic monohydrate,
about 0.015% w/v polysorbate 80,
about 150 mM NaCl,
about 5 mM Tris-HCl, and
about 5% w/v sucrose,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the pH of the composition is about 7.4. In embodiments, the HA-ss-np comprises an A1-ss-np and an A2-ss-np, the CpG ODN is CPG 7909, and the pH of the composition is about 7.4.

In embodiments, the vaccine composition comprises
about 0.008% w/v or about 0.024% w/v of HA-ss-np,
about 0.06% w/v of aluminum hydroxide,
about 0.05% w/v of CpG ODN,
about 5 mM of sodium phosphate monobasic monohydrate,
about 0.015% w/v of a polysorbate 80,
about 150 mM NaCl,
about 5 mM Tris-HCl, and
about 5% w/v of sucrose,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the pH of the composition is about 7.4. In embodiments, the HA-ss-np comprises an A1-ss-np and an A2-ss-np, the CpG ODN is CPG 7909, and the pH of the composition is about 7.4.

In embodiments, the vaccine composition comprises
about 0.08 mg/mL or about 0.24 mg/mL of HA-ss-np,
about 0.6 mg/mL of aluminum hydroxide,
about 0.05% w/v of CpG ODN,
about 5 mM of sodium phosphate monobasic monohydrate,
about 0.015% w/v polysorbate 80,
about 150 mM NaCl,
about 5 mM Tris-HCl, and
about 5% w/v sucrose,
   wherein the HA-ss-np is not substantially adsorbed to the aluminum hydroxide, and wherein at least a portion of the CpG ODN is adsorbed to the aluminum hydroxide in the composition. In embodiments, the HA-ss-np comprises an A1-ss-np and an A2-ss-np. In embodiments, the CpG ODN is CPG 7909. In embodiments, the pH of the composition is about 7.4. In embodiments, the HA-ss-np comprises an A1-ss-np and an A2-ss-np, the CpG ODN is CPG 7909, and the pH of the composition is about 7.4.

In embodiments, the vaccine composition comprises
about 0.0005% w/v to about 0.1% w/v of each of A1-ss-np and A2-ss-np,
about 0.01% w/v to about 1% w/v of aluminum hydroxide,
about 0.01% w/v to about 0.5% w/v of CpG ODN,
about 0.2 mM to about 4 mM of sodium phosphate monobasic monohydrate,
about 0.005% w/v to about 0.05% w/v of polysorbate 80,
about 10 mM to about 500 mM NaCl,
about 1 mM to about 10 mM of Tris-HCl, and
about 1% w/v to about 25% w/v of sucrose,
   wherein the A1-ss-np and the A2-ss-np are not substantially adsorbed to the aluminum hydroxide, and wherein at least a portion of the CpG ODN is adsorbed to the aluminum hydroxide in the composition. In embodiments, the CpG ODN is CPG 7909. In embodiments, the pH of the composition is about 7.4. In embodiments, the CpG ODN is CPG 7909 and the pH of the composition is about 7.4.

In embodiments, the vaccine composition comprises
about 0.0004% w/v to about 0.015% w/v of each of A1-ss-np and A2-ss-np,
about 0.05% w/v to about 0.07% w/v of aluminum hydroxide,
about 0.06% w/v to about 0.12% w/v of CpG ODN,
about 0.3 mM to about 0.4 mM of sodium phosphate monobasic monohydrate,
about 0.010% w/v to about 0.02% w/v of polysorbate 80,
about 50 mM to about 200 mM NaCl,
about 1 mM to about 10 mM Tris-HCl, and
about 1% w/v to about 15% w/v sucrose,
   wherein the A1-ss-np and the A2-ss-np are not substantially adsorbed to the aluminum hydroxide, and wherein at least a portion of the CpG ODN is adsorbed to the aluminum hydroxide in the composition. In embodiments, the CpG ODN is CPG 7909. In embodiments, the pH of the composition is about 7.4. In embodiments, the CpG ODN is CPG 7909 and the pH of the composition is about 7.4.

In embodiments, the vaccine composition comprises
about 0.0005% w/v to about 0.1% w/v of each of A1-ss-np and A2-ss-np,
about 0.010% w/v to about 1% w/v of aluminum hydroxide,
about 0.01% w/v to about 0.5% w/v of CpG ODN,
about 0.2 mM to about 20 mM of sodium phosphate monobasic monohydrate,
about 0.005% w/v to about 0.05% w/v of polysorbate 80,
about 10 mM to about 500 mM NaCl,
about 1 mM to about 10 mM of Tris-HCl, and
about 1% w/v to about 25% w/v of sucrose,
   wherein the A1-ss-np and the A2-ss-np are not substantially adsorbed to the aluminum hydroxide, and wherein at least a portion of the CpG ODN is adsorbed to the aluminum hydroxide in the composition. In embodiments, the CpG ODN is CPG 7909. In embodiments, the pH of the composition is about 7.4. In embodiments, the CpG ODN is CPG 7909 and the pH of the composition is about 7.4.

In embodiments, the vaccine composition comprises
about 0.0004% w/v to about 0.015% w/v of each of A1-ss-np and A2-ss-np,
about 0.05% w/v to about 0.07% w/v of aluminum hydroxide,
about 0.02% w/v to about 0.08% w/v of CpG ODN,
about 2 mM to about 8 mM of sodium phosphate monobasic monohydrate,
about 0.010% w/v to about 0.02% w/v of polysorbate 80,
about 50 mM to about 200 mM NaCl,
about 1 mM to about 10 mM Tris-HCl, and
about 1% w/v to about 15% w/v sucrose,
   wherein the A1-ss-np and the A2-ss-np are not substantially adsorbed to the aluminum hydroxide, and wherein at least a portion of the CpG ODN is adsorbed to the aluminum hydroxide in the composition. In embodiments, the CpG ODN is CPG 7909. In embodiments, the pH of the composition is about 7.4. In embodiments, the CpG ODN is CPG 7909 and the pH of the composition is about 7.4.

In embodiments, the vaccine composition comprises
about 0.004% w/v or about 0.012% w/v of each of A1-ss-np and A2-ss-np,
about 0.06% w/v of aluminum hydroxide,
about 0.1% w/v of CpG ODN,
about 0.36 mM of sodium phosphate monobasic monohydrate,
about 0.015% w/v of a polysorbate 80,
about 150 mM NaCl,
about 5 mM Tris-HCl, and
about 5% w/v of sucrose,
   wherein the A1-ss-np and the A2-ss-np are not substantially adsorbed to the aluminum hydroxide, and wherein at least a portion of the CpG ODN is adsorbed to the aluminum hydroxide in the composition. In embodiments, the CpG ODN is CPG 7909. In embodiments, the pH of the composition is about 7.4. In embodiments, the CpG ODN is CPG 7909 and the pH of the composition is about 7.4.

In embodiments, the vaccine composition comprises
about 0.04 mg/mL or about 0.12 mg/mL of each of A1-ss-np and A2-ss-np,
about 0.6 mg/mL of aluminum hydroxide,
about 1 mg/mL of CpG ODN, and
about 0.36 mM sodium phosphate monobasic monohydrate,
about 0.015% w/v polysorbate 80,
about 150 mM NaCl,
about 5 mM Tris-HCl, and
about 5% w/v sucrose,
   wherein the A1-ss-np and the A2-ss-np are not substantially adsorbed to the aluminum hydroxide, and wherein at least a portion of the CpG ODN is adsorbed to the aluminum hydroxide in the composition. In embodiments, the CpG ODN is CPG 7909. In embodiments, the pH of the composition is about 7.4. In embodiments, the CpG ODN is CPG 7909 and the pH of the composition is about 7.4.

In embodiments, the vaccine composition comprises
about 0.004% w/v or about 0.012% w/v of each of A1-ss-np and A2-ss-np,
about 0.06% w/v of aluminum hydroxide,
about 0.05% w/v of CpG ODN,
about 5 mM of sodium phosphate monobasic monohydrate,
about 0.015% w/v of a polysorbate 80,
about 150 mM NaCl,
about 5 mM Tris-HCl, and
about 5% w/v of sucrose,
   wherein the A1-ss-np and the A2-ss-np are not substantially adsorbed to the aluminum hydroxide, and wherein at least a portion of the CpG ODN is adsorbed to the aluminum hydroxide in the composition. In embodiments, the CpG ODN is CPG 7909. In embodiments, the pH of the composition is about 7.4. In embodiments, the CpG ODN is CPG 7909 and the pH of the composition is about 7.4.

In embodiments, the vaccine composition comprises
about 0.04 mg/mL or about 0.12 mg/mL of each of A1-ss-np and A2-ss-np,
about 0.6 mg/mL of aluminum hydroxide,
about 0.05% w/v of CpG ODN,
about 5 mM of sodium phosphate monobasic monohydrate,
about 0.015% w/v polysorbate 80,
about 150 mM NaCl,
about 5 mM Tris-HCl, and
about 5% w/v sucrose,
   wherein the A1-ss-np and the A2-ss-np are not substantially adsorbed to the aluminum hydroxide, and wherein at least a portion of the CpG ODN is adsorbed to the aluminum hydroxide in the composition. In embodiments, the CpG ODN is CPG 7909. In embodiments, the pH of the composition is about 7.4. In embodiments, the CpG ODN is CPG 7909 and the pH of the composition is about 7.4.

In embodiments, the vaccine composition further comprises a pharmaceutically acceptable carrier. In embodiments, the pharmaceutically acceptable carrier comprises a sterile liquid. In embodiments, the pharmaceutically acceptable carrier comprises water. In embodiments, the pharmaceutically acceptable carrier comprises saline solution. In embodiments, the pharmaceutically acceptable carrier comprises an oil. Exemplary pharmaceutically acceptable oil carriers include, but are not limited to, petroleum, synthetic oil, animal oil, or vegetable oil, such as peanut oil, soybean oil, mineral oil, and sesame oil.

### Methods

In embodiments, the invention provides a method of inducing an immunological response against an influenza virus in a subject in need thereof, comprising administering an immunologically effective amount of the vaccine composition provided herein to the subject. The terms “immunologically effective amount” and “immunogenic amount,” which are used interchangeably herein, refer to the amount of antigen or vaccine composition sufficient to elicit an immune response described herein, e.g., a cellular (T cell) or humoral (B cell) response, or both, as measured by standard assays known to one of ordinary skill in the art.

For example, the effectiveness of an antigen as an immunogen can be measured by measuring the levels of B cell activity by measuring the levels of circulating antibodies specific for the antigen in serum using immunoassays, immunoprecipitation assays, and functional antibody assays, such as in vitro opsonic assays. Another measure of effectiveness of an antigen as a T-cell immunogen can be measured by proliferation assays or by cytolytic assays, such as chromium release assays to measure the ability of a T cell to lyse its specific target cell. In embodiments, an immunogenic amount is defined by measuring the serum levels of antigen specific antibody induced following administration of the antigen, or, by measuring the ability of the antibodies so induced to enhance the opsonophagocytic ability of particular white blood cells. The level of protection of the immune response may be measured by challenging the immunized host with the antigen that has been injected. For example, if the antigen to which an immune response is desired is a virus, the level of protection induced by the immunogenic amount of the antigen can be measured by detecting the percent survival or the percent mortality after challenge of the animals with the virus. In one embodiment, the amount of protection may be measured by measuring at least one symptom associated with the viral infection, for example, a fever associated with the infection. In embodiments, the immunogenic amount of the vaccine composition is determined from a dose response study in which subjects are immunized with gradually increasing amounts of the immunogenic composition and the immune response analyzed to determine the optimal dosage.

In embodiments, the immunologically effective amount of the HA-ss-np antigen is about 10 μg to about 500 μg, about 15 μg to about 450 μg, about 20 μg to about 400 μg, about 30 μg to about 350 μg, about 40 μg to about 300 μg, about 50 μg to about 280 μg, about 60 to about 260 μg, or about 80 μg to about 240 μg. In embodiments, the immunologically effective amount of the HA-ss-np antigen is about 10, 20, 30, 40, 50, 60, 70, 80, 90, 100, 120, 140, 160, 180, 200, 220, 240, 260, 280, or 300 μg.

In embodiments, the invention provides a method of inducing an immunological response against an influenza virus in a subject in need thereof, comprising administering a dose of about 20 μg to about 300 μg of an HA-ss-np in a vaccine composition, wherein the vaccine composition further comprises an aluminum hydroxide; CpG ODN; and a phosphate salt, and wherein the HA-ss-np is not substantially adsorbed to the aluminum hydroxide, and wherein at least a portion of the CpG ODN is adsorbed to the aluminum hydroxide. Components of the vaccine composition are provided herein.

In embodiments, the method comprises administering about 10 μg to about 150 μg of a first HA-ss-np comprising an A1-ss-np; and/or about 10 μg to about 150 μg of a second HA-ss-np comprising an A2-ss-np. In embodiments, the method comprises administering about 20 μg to about 140 μg of an A1-ss-np; and/or about 20 μg to about 140 μg of an A2-ss-np. In embodiments, the method comprises administering about 30 μg to about 120 μg of an A1-ss-np; and/or about 30 μg to about 120 μg of an A2-ss-np. In embodiments, the method comprises administering about 30 μg A1-ss-np and/or about 30 μg A2-ss-np. In embodiments, the method comprises administering about 120 μg A1-ss-np and/or about 120 μg A2-ss-np.

In embodiments, the subject is a mammalian subject. In embodiments, the subject is a human subject. In embodiments, the subject is particularly at risk of or susceptible to influenza virus infection, including, for example, an immunocompromised subject. In embodiments, the subject is in an age group that is at elevated risk for influenza virus infection, e.g., children younger than 2 years old or adults older than 65 years old. In embodiments, the subject is an animal subject, e.g., an animal model such as a mouse, rat, rabbit, or nonhuman primate, or a veterinary or farm animal, e.g., dog, cat, bird, horse, cattle, sheep, or chicken.

In embodiments, the dose is formulated for administration to a human subject. In embodiments, the dose is formulated for administration to an adult, teen, adolescent, toddler, or infant (i.e., no more than one year old) human subject.

In embodiments, the method reduces the transmission of the influenza virus by an infected individual, e.g., by reducing the amount of viral shedding. In embodiments, the method prevents or reduces the severity of at least one symptom associated with an influenza infection, e.g., fever, cough, sore throat, fatigue, vomiting, diarrhea, sinus infection, ear infection, and the like. In embodiments, the method prevents or reduces the likelihood of the subject developing clinical complications associated with an influenza infection, e.g., significant weight loss, pneumonia, inflammation of the heart, brain, or muscle tissues, multi-organ failure, and/or sepsis. In embodiments, the method reduces the risk of death associated with an influenza infection.

In embodiments, the method comprises administering a single dose of the vaccine composition. In embodiments, the method comprises annually administering a single dose of the vaccine composition. In embodiments, the method comprises administering two or more doses of the vaccine composition. In embodiments, the method comprises annually administering two or more doses of the vaccine composition. In embodiments, the method comprises administering two or more doses of the vaccine composition, wherein a second dose is administered about 1 day to about 3 months, about 3 days to about 2 months, 1 week to about 6 weeks, or about 2 weeks to about 4 weeks after a first dose. In embodiments, the method comprises administering two or more doses of the vaccine composition, wherein a second dose is administered about 1 day, about 3 days, about 5 days, about 1 week, about 2 weeks, about 3 weeks, about 4 weeks, about 5 weeks, about 6 weeks, about 7 weeks, about 8 weeks, about 10 weeks, or about 12 weeks after a first dose.

In embodiments, the vaccine composition provided herein is administered to the subject via parenteral administration (intramuscularly, intraperitoneally, intradermally, subcutaneously, intravenously, or to the interstitial space of a tissue); or via rectal, oral, vaginal, topical, transdermal, intranasal, ocular, aural, pulmonary or other mucosal administration. In embodiments, parenteral administration is by intramuscular injection, e.g., to the thigh or upper arm of the subject. In embodiments, the injection is via a needle (e.g., a hypodermic needle). In embodiments, the injection is needle free injection. In embodiments, the intramuscular dose is about 0.1 mL to about 5 mL, about 0.2 mL to about 1 mL, about 0.3 mL to about 0.8 mL, about 0.4 mL to about 0.6 mL, or about 0.5 mL.

In embodiments, the vaccine composition is a liquid solution or suspension for injection. In embodiments, the vaccine composition is a powder or spray for pulmonary administration, e.g., in an inhaler. In embodiments, the composition is a suppository or pessary, or for nasal, aural or ocular administration, e.g., as a spray, drops, gel or powder.

In embodiments, the invention provides a method of producing a vaccine composition, comprising: combining HA-ss-np with an adjuvant mixture, wherein the adjuvant mixture comprises a diluent solution comprising a phosphate salt; aluminum hydroxide; and CpG ODN, wherein the CpG ODN is in an excess amount relative to the aluminum hydroxide, wherein the adjuvant mixture comprises CpG ODN-adsorbed aluminum hydroxide, and wherein the HA-ss-np is not substantially adsorbed to the aluminum hydroxide. Components of the vaccine composition are further described herein.

In embodiments, the adjuvant mixture is prepared at least 5 minutes, at least 10 minutes, at least 15 minutes, at least 30 minutes, at least 1 hour, at least 2 hours, at least 4 hours, at least 6 hours, at least 12 hours, at least 1 day, at least 2 days, at least 3 days, at least 1 week, at least 2 weeks, at least 1 month, at least 3 months, at least 6 months, at least 9 months, at least 12 months, or greater than 12 months prior to the combining step. In embodiments, the adjuvant mixture is stable when stored at about −80° C. to about 40° C., or about −70° C. to about 30° C., or about −60° C. to about 20° C., or about −50° C. to about 10° C., or about −40° C. to about 0° C., or about −30° C. to about −10° C., or about 0° C. to about 30° C., or about 10° C. to about 20° C., or about 20° C. to about 25° C., for about 5 minutes to more than 12 months, about 10 minutes to about 9 months, about 15 minutes to about 6 months, about 30 minutes to about 3 months, about 1 hour to about 1 month, about 2 hours to about 2 weeks, about 4 hours to about 1 week, about 6 hours to about 3 days, or about 12 hours to about 2 days.

In embodiments, the combining occurs about at least 5 minutes, at least 10 minutes, at least 15 minutes, at least 30 minutes, at least 1 hour, at least 2 hours, at least 4 hours, at least 6 hours, at least 12 hours, at least 1 day, at least 2 days, at least 3 days, at least 1 week, at least 2 weeks, at least 1 month, at least 3 months, at least 6 months, at least 9 months, at least 12 months, or greater than 12 months prior to administering the vaccine composition to the subject, e.g., as described herein. In embodiments, the method comprises producing the vaccine composition as described herein, storing the vaccine composition, e.g., for about 5 minutes to more than 12 months, about 10 minutes to about 9 months, about 15 minutes to about 6 months, about 30 minutes to about 3 months, about 1 hour to about 1 month, about 2 hours to about 2 weeks, about 4 hours to about 1 week, about 6 hours to about 3 days, or about 12 hours to about 2 days, prior to administering the vaccine composition to a subject as described herein.

In embodiments, the HA-ss-np is provided as a solid, the adjuvant mixture is provided as a liquid, and the combining comprises mixing and/or solubilizing the HA-ss-np into the adjuvant mixture. In embodiments, the HA-ss-np and the adjuvant mixture are provided as a liquid, and the combining comprises mixing the HA-ss-np and the adjuvant mixture. In embodiments, the method further comprises lyophilizing the vaccine composition. Methods of lyophilization are known to one of ordinary skill in the art. In embodiments, the vaccine composition is provided in a kit comprising two or more vials, a pre-filled and/or empty syringe, a needle and/or a container, wherein the lyophilized composition is reconstituted prior to administration, e.g., injection.

In embodiments, the method further comprises packaging the vaccine composition is packaged in unit dose or multi-dose form (e.g. 2 doses, 4 doses, 6 doses, 8 doses, or more). In embodiments, the multi-dose form comprises a vial or a pre-filled delivery devices, e.g., single or multiple component syringes.

In embodiments, the invention further provides a vaccine composition kit comprising a first component that comprises a stabilized, dry (e.g., lyophilized) vaccine composition provided herein, and a second component comprising a sterile, aqueous solution for reconstitution of the first component. In embodiments, the first component comprises the HA-ss-np antigen, and the second component comprises the aluminum hydroxide, CpG ODN, and phosphate salt as described herein.

In embodiments, the invention provides a vaccine composition kit comprising in separate vials or containers: (1) an antigen composition comprising an HA-ss-np antigen (about 2.4 mg/mL), about 5 mM Tris, about 150 mM NaCl, and about 5% w/v sucrose, at a pH of about 7.4; (2) a CPG ODN composition comprising CPG ODN (about 1.19 mg/mL), about 5 mM Tris, about 150 mM NaCl, about 5% w/v sucrose, about 0.015% PS-80, and about 0.4 mM sodium phosphate at a pH of about 7.4; (3) a diluent composition comprising about 5 mM Tris, about 150 mM NaCl,
about 5% w/v sucrose at a pH of about 7.4; (4) an aluminum hydroxide composition comprising an about 2% aluminum hydroxide suspension in deionized water.

All references cited herein, including patents, patent applications, papers, textbooks and the like, and the references cited therein, to the extent that they are not already, are hereby incorporated herein by reference in their entirety.

### Sequences

SEQ ID NO: 1. Exemplary amino acid sequence of an HA-ss-np comprising an HA-ss from
an H1 influenza virus.
MKAKLLVLLCTFTATYADTICIGYHANNSTDTVDTVLEKNVTVTHSVNLGSGLRMVTGLRNIPQRETRGLFGAIAGFIE
GGWTGMVDGWYGYHHQNEQGSGYAADQKSTQNAINGITNMVNSVIEKMGSGGSGTDLAELLVLLLNERTLDFHDSNVKN
LYEKVKSQLKNNAKEIGNGCFEFYHKCNNECMESVKNGTYDYPKYSEESKLNREKIDSGGDIIKLLNEQVNKEMQSSNL
YMSMSSWCYTHSLDGAGLFLEDHAAEEYEHAKKLIIFLNENNVPVQLTSISAPEHKFEGLTQIFQKAYEHEQHISESIN
NIVDHAIKSKDHATFNFLQWYVAEQHEEEVLFKDILDKIELIGNENHGLYLADQYVKGIAKSRKSGS
SEQ ID NO: 2. Exemplary amino acid sequence of an HA-ss-np comprising an HA-ss from
an H1 influenza virus.
MKAKLLVLLCTFTATYADTICIGYHANNSTDTVDTVLEKNVTVTHSVNLGSGLRMVTGLRNIPQRETRGLFGAIAGFIE
GGWTGMVDGWYGYHHQNEQGSGYAADQKSTQNAINGITNMVNSVIEKMGSGGSGTDLAELLVLLLNERTLDFHDSNVKN
LYEKVKSQLKNNAKEIGNGCFEFYHKCNNECMESVKNGTYDYPKYSEESKLNREKIDSGGDIIKLLNEQVNKEMQSSNL
YMSMSSWCYTHSLDGAGLFLFDHAAEEYEHAKKLIIFLNENNVPVQLTSISAPEHKFEGLTQIFQKAYEHEQHISESIN
NIVDHAIKSKDHATFNFLQWYVAEQHEEEVLFKDILDKIELIGNENHGLYLADQYVKGIAKSRKS
SEQ ID NO: 3. Exemplary amino acid sequence of an HA-ss-np comprising an HA-ss from
an H10 influenza virus.
MYKIVVIIALLGAVKGLDKICLGHHAVANGTIVKTLTNEQEEVTNATELVFPGCGVLMLATGMRNVPELIQGRGLFGAI
AGFLENGWEGMVDGWYGFRHQNAQGTGQAADYKSTQAAIDQITGMVNRVVELMEQGGPDCYLAELLVAMLNQHVIDMAD
SEMRNLYERVRKQLRQNAEEDGKGCFEIYHACDDSCMESIRNNTYDHSQYREEALLNRLNINSGGDIIKLLNEQVNKEM
QSSNLYMSMSSWCYTHSLDGAGLFLFDHAAEEYEHAKKLIIFLNENNVPVQLTSISAPEHKFEGLTQIFQKAYEHEQHI
SESINNIVDHAIKSKDHATFNFLQWYVAEQHEEEVLFKDILDKIELIGNENHGLYLADQYVKGIAKSRKSGS
SEQ ID NO: 4. Exemplary amino acid sequence of an HA-ss-np comprising an HA-ss from
an H10 influenza virus.
MYKIVVIIALLGAVKGLDKICLGHHAVANGTIVKTLTNEQEEVTNATELVFPGCGVLMLATGMRNVPELIQGRGLFGAI
AGFLENGWEGMVDGWYGFRHQNAQGTGQAADYKSTQAAIDQITGMVNRVVELMEQGGPDCYLAELLVAMLNQHVIDMAD
SEMRNLYERVRKQLRQNAEEDGKGCFEIYHACDDSCMESIRNNTYDHSQYREEALLNRLNINSGGDIIKLLNEQVNKEM
QSSNLYMSMSSWCYTHSLDGAGLELFDHAAEEYEHAKKLIIFLNENNVPVQLTSISAPEHKFEGLTQIFQKAYEHEQHI
SESINNIVDHAIKSKDHATFNFLQWYVAEQHEEEVLEKDILDKIELIGNENHGLYLADQYVKGIAKSRKS
SEQ ID NO: 5. Exemplary amino acid sequence of an HA-ss-np comprising an HA-ss from
an H1 influenza virus.
DTICIGYHANNSTDTVDTVLEKNVTVTHSVNLGSGLRMVTGLRNIPQRETRGLFGAIAGFIEGGWTGMVDGWYGYHHQN
EQGSGYAADQKSTQNAINGITNMVNSVIEKMGSGGSGTDLAELLVLLLNERTLDFHDSNVKNLYEKVKSQLKNNAKEIG
NGCFEFYHKCNNECMESVKNGTYDYPKYSEESKLNREKIDSGGDIIKLLNEQVNKEMQSSNLYMSMSSWCYTHSLDGAG
LFLFDHAAEEYEHAKKLIIFLNENNVPVQLTSISAPEHKFEGLTQIFQKAYEHEQHISESINNIVDHAIKSKDHATFNF
LQWYVAEQHEEEVLEKDILDKIELIGNENHGLYLADQYVKGIAKSRKSGS
SEQ ID NO: 6. Exemplary amino acid sequence of an HA-ss-np comprising an HA-ss from
an H1 influenza virus.
DTICIGYHANNSTDTVDTVLEKNVTVTHSVNLGSGLRMVTGLRNIPQRETRGLFGAIAGFIEGGWTGMVDGWYGYHHQN
EQGSGYAADQKSTQNAINGITNMVNSVIEKMGSGGSGTDLAELLVLLLNERTLDFHDSNVKNLYEKVKSQLKNNAKEIG
NGCFEFYHKCNNECMESVKNGTYDYPKYSEESKLNREKIDSGGDIIKLLNEQVNKEMQSSNLYMSMSSWCYTHSLDGAG
LFLFDHAAEEYEHAKKLIIFLNENNVPVQLTSISAPEHKFEGLTQIFQKAYEHEQHISESINNIVDHAIKSKDHATFNF
LQWYVAEQHEEEVLFKDILDKIELIGNENHGLYLADQYVKGIAKSRKS
SEQ ID NO: 7. Exemplary amino acid sequence of an HA-ss-np comprising an HA-ss from
an H10 influenza virus.
LDKICLGHHAVANGTIVKTLTNEQEEVTNATELVFPGCGVLMLATGMRNVPELIQGRGLFGAIAGFLENGWEGMVDGWY
GFRHQNAQGTGQAADYKSTQAAIDQITGMVNRVVELMEQGGPDCYLAELLVAMLNQHVIDMADSEMRNLYERVRKQLRQ
NAEEDGKGCFEIYHACDDSCMESIRNNTYDHSQYREEALLNRLNINSGGDIIKLLNEQVNKEMQSSNLYMSMSSWCYTH
SLDGAGLFLFDHAAEEYEHAKKLIIFLNENNVPVQLTSISAPEHKFEGLTQIFQKAYEHEQHISESINNIVDHAIKSKD
HATFNFLQWYVAEQHEEEVLFKDILDKIELIGNENHGLYLADQYVKGIAKSRKSGS
SEQ ID NO: 8. Exemplary amino acid sequence of an HA-ss-np comprising an HA-ss from
an H10 influenza virus.
LDKICLGHHAVANGTIVKTLTNEQEEVTNATELVFPGCGVLMLATGMRNVPELIQGRGLFGAIAGFLENGWEGMVDGWY
GFRHQNAQGTGQAADYKSTQAAIDQITGMVNRVVELMEQGGPDCYLAELLVAMLNQHVIDMADSEMRNLYERVRKQLRQ
NAEEDGKGCFEIYHACDDSCMESIRNNTYDHSQYREEALLNRLNINSGGDIIKLLNEQVNKEMQSSNLYMSMSSWCYTH
SLDGAGLFLFDHAAEEYEHAKKLIIFLNENNVPVQLTSISAPEHKFEGLTQIFQKAYEHEQHISESINNIVDHAIKSKD
HATFNFLQWYVAEQHEEEVLEKDILDKIELIGNENHGLYLADQYVKGIAKSRKS
SEQ ID NO: 9. Exemplary amino acid sequence of an HA-ss from an H1 influenza virus.
MKAKLLVLLCTFTATYADTICIGYHANNSTDTVDTVLEKNVTVTHSVNLGSGLRMVTGLRNIPQRETRGLFGAIAGFIE
GGWTGMVDGWYGYHHQNEQGSGYAADQKSTQNAINGITNMVNSVIEKMGSGGSGTDLAELLVLLLNERTLDFHDSNVKN
LYEKVKSQLKNNAKEIGNGCFEFYHKCNNECMESVKNGTYDYPKYSEESKLNREKID
SEQ ID NO: 10. Exemplary amino acid sequence of an HA-ss from an H10 influenza virus.
MYKIVVIIALLGAVKGLDKICLGHHAVANGTIVKTLTNEQEEVTNATELVFPGCGVLMLATGMRNVPELIQGRGLFGAI
AGFLENGWEGMVDGWYGFRHQNAQGTGQAADYKSTQAAIDQITGMVNRVVELMEQGGPDCYLAELLVAMLNQHVIDMAD
SEMRNLYERVRKQLRQNAEEDGKGCFEIYHACDDSCMESIRNNTYDHSQYREEALLNRLNIN
SEQ ID NO: 11. Exemplary amino acid sequence of an HA-ss from an H1 influenza virus.
DTICIGYHANNSTDTVDTVLEKNVTVTHSVNLGSGLRMVTGLRNIPQRETRGLFGAIAGFIEGGWTGMVDGWYGYHHQN
EQGSGYAADQKSTQNAINGITNMVNSVIEKMGSGGSGTDLAELLVLLLNERTLDFHDSNVKNLYEKVKSQLKNNAKEIG
NGCFEFYHKCNNECMESVKNGTYDYPKYSEESKLNREKID
SEQ ID NO: 12. Exemplary amino acid sequence of an HA-ss from an H10 influenza virus.
LDKICLGHHAVANGTIVKTLTNEQEEVTNATELVFPGCGVLMLATGMRNVPELIQGRGLFGAIAGFLENGWEGMVDGWY
GFRHQNAQGTGQAADYKSTQAAIDQITGMVNRVVELMEQGGPDCYLAELLVAMLNQHVIDMADSEMRNLYERVRKQLRQ
NAEEDGKGCFEIYHACDDSCMESIRNNTYDHSQYREEALLNRLNIN
SEQ ID NO: 13. Exemplary amino acid sequence of ferritin.
DIIKLLNEQVNKEMQSSNLYMSMSSWCYTHSLDGAGLFLEDHAAEEYEHAKKLIIFLNENNVPVQLTSISAPEHKFEGL
TQIFQKAYEHEQHISESINNIVDHAIKSKDHATFNFLQWYVAEQHEEEVLFKDILDKIELIGNENHGLYLADQYVKGIA
KSRKSGS
SEQ ID NO: 14. Exemplary amino acid sequence of ferritin.
DIIKLLNEQVNKEMQSSNLYMSMSSWCYTHSLDGAGLFLEDHAAEEYEHAKKLIIFLNENNVPVQLTSISAPEHKFEGL
TQIFQKAYEHEQHISESINNIVDHAIKSKDHATFNFLQWYVAEQHEEEVLFKDILDKIELIGNENHGLYLADQYVKGIA
KSRKS
SEQ ID NO:15. Exemplary amino acid linker sequence.
SGG
SEQ ID NO: 16. Exemplary polynucleotide sequence of CPG 7909.
TCG TCG TTT TGT CGT TTT GTC GTT

### EXAMPLES

### Example 1. Assessment of H1-Ss-Np with Adjuvant Combinations

Formulations of a Type A, subtype H1 influenza hemagglutinin stabilized stem ferritin nanoparticle (“H1-ss-np”) in combination with different adjuvants were administered intramuscularly to BALB/c mice and assessed for protective immunity. Survival and weight loss following intranasal challenge with mouse-adapted influenza A/CA/04/09 were the primary endpoints. Serum samples were collected at designated time points following immunization and analyzed.

### Materials and Methods

Animals: Female 6-week-old BALB/c mice were obtained from Charles River Laboratories (Wilmington, MA) for this experiment. The mice were quarantined for 7 days before use and maintained on Teklad Rodent Diet (Harlan Teklad) and tap water at the Laboratory Animal Research Center of Utah State University.

Virus: Influenza A/CA/04/2009 (pandemic H1N1) was used for lethal challenge. Influenza A/CA/04/2009 (H1N1), strain designation 175190, was received from St. Jude Children's Research Hospital, Memphis TN. The virus was adapted to replication in the lungs of BALB/c mice by 9 sequential passages in mice. Virus was plaque purified in MDCK cells and a virus stock was prepared by growth in embryonated chicken eggs and then MDCK cells.

Vaccine and Adjuvants: Antigen: H1-ss-np vaccine; Adjuvants: CpG 1826, Alhydrogel®, Advax® (inulin), AddaVax™ (squalene-based), Monophsophoryl Lipid A (MPLA), PolyL:PolyC (Hiltonol®), Quil-A®, Resiquimod and the Sigma Adjuvant System (SAS®). In formulations that included Alhydrogel and an additional adjuvant, the additional adjuvant was adsorbed onto the Alhydrogel in saline for several days, minimum of 48 hours and up to seven days such that no additional binding/adsorption capacity was expected to remain for any other components (i.e., antigen). No additional phosphate was added. The H1-ss-np was added/mixed with the Alhydrogel/adjuvant solution immediately prior to injection.

Experiment desi n: A total of 235 mice were randomized into 23 groups of 10 (except placebo group 23 that was comprised of 15 mice) mice per group (Table 1). Mice in each group were vaccinated on Days 0 and 21 by intramuscular (IM) injection of 50 μL per quadriceps (total 100 μL vaccine per mouse) of the designated antigen/adjuvant combination.

TABLE 1
Example 1 Experiment Design

| | | | | Vaccination | Challenge | Bleed | |
|---|---|---|---|---|---|---|---|
| | | Ag Dose | | Schedule | Schedule | Schedule | No. of |
| Group | Ag | (μg) | Adjuvant | (Days) | (Day) | (Days) | Mice |
| 1 | H1- | 0.1 | Alhydrogel (20 μg) | 0, 21 | 49 | 0, 21, | 10 |
| 2 | ss-np | 1 | | | | 49, 70 | 10 |
| 3 | | 0.1 | CpG 1826 (20 μg) | | | (terminal) | 10 |
| 4 | | 1 | | | | | 10 |
| 5 | | 0.1 | CpG 1826 (20 μg) and | | | | 10 |
| 6 | | 1 | Alhydrogel (20 μg) | | | | 10 |
| 7 | | 0.1 | Advax (Inulin) (20 μg) | | | | 10 |
| 8 | | 1 | | | | | 10 |
| 9 | | 0.1 | AddaVax (1:1 v/v) | | | | 10 |
| 10 | | 1 | | | | | 10 |
| 11 | | 0.1 | MPLA (15 μg) | | | | 10 |
| 12 | | 1 | | | | | 10 |
| 13 | | 0.1 | Alhydrogel (20 μg) | | | | 10 |
| 14 | | 1 | and MPLA (15 μg) | | | | 10 |
| 15 | | 0.1 | PolyI:PolyC (20 μg) | | | | 10 |
| 16 | | 1 | | | | | 10 |
| 17 | | 0.1 | Quil-A (20 μg) | | | | 10 |
| 18 | | 1 | | | | | 10 |
| 19 | | 0.1 | Resiquimod (2.5 μg) | | | | 10 |
| 20 | | 1 | | | | | 10 |
| 21 | | 0.1 | SAS | | | | 10 |
| 22 | | 1 | | | | | 10 |

| | | | |
|---|---|---|---|
| 23 | Placebo | None | 15 |
| | | | 235 |

Mice were challenged with mouse-adapted influenza A/California/04/2009(H1N1) (A/CA/04/09; H1N1pdm) via intranasal route on day 49. For challenge, mice were anesthetized by intraperitoneal (i.p.). injection of ketamine/xylazine (50 mg/kg//5 mg/kg) prior to challenge by the intranasal route with approximately 1×10⁵ (3×LD₅₀) cell culture infectious doses (CCID₅₀) of virus per mouse in a 90 μL inoculum volume. Mice were weighed prior to treatment and then every other day thereafter to assess the effects of treatment on ameliorating weight loss due to virus infection. All mice were observed for morbidity and mortality through day 21 post challenge.

Non-terminal collection of 0.1 to 0.2 mL of blood was completed on all animals on days 0 (baseline), 21, and 49. The terminal bleed via cardiac puncture (exsanguination) was completed on all surviving animals on day 70. Serum samples were prepared and frozen at −20° C. prior to serological analyses.

Statistical analysis: Kaplan-Meier survival curves were generated and compared by the Log-rank (Mantel-Cox) test followed by pairwise comparison using the Gehan-Breslow-Wilcoxon test in Prism 7.0c (GraphPad Software Inc.). Mean body weights were analyzed by one-way analysis of variance (ANOVA) followed by Tukey's multiple comparison tests using Prism 7.0c.

Ethics regulation of laboratory animals: This study was conducted in accordance with applicable regulations regarding the ethical treatment of laboratory animals.

### Results

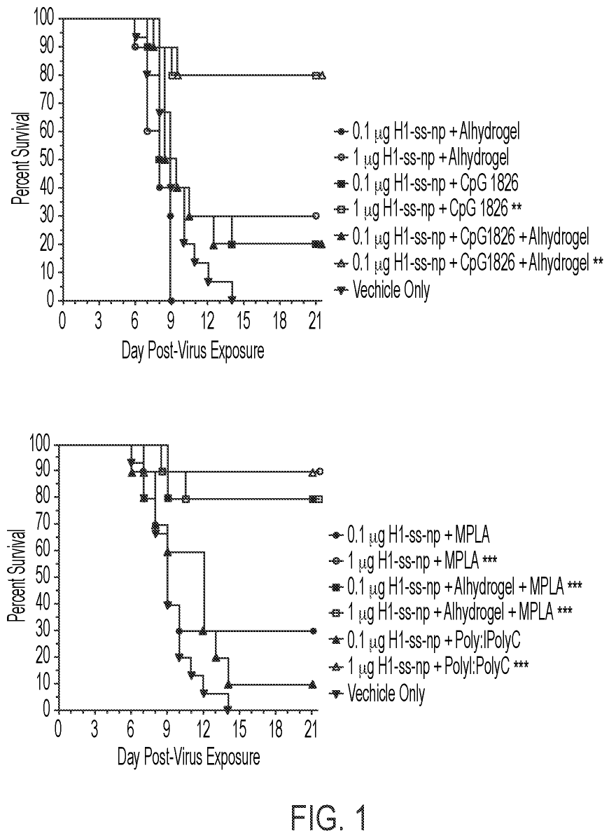

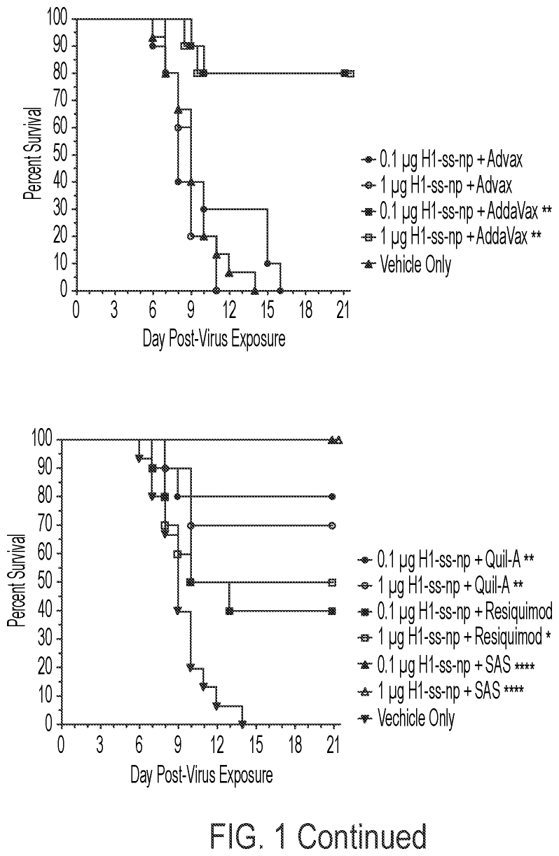

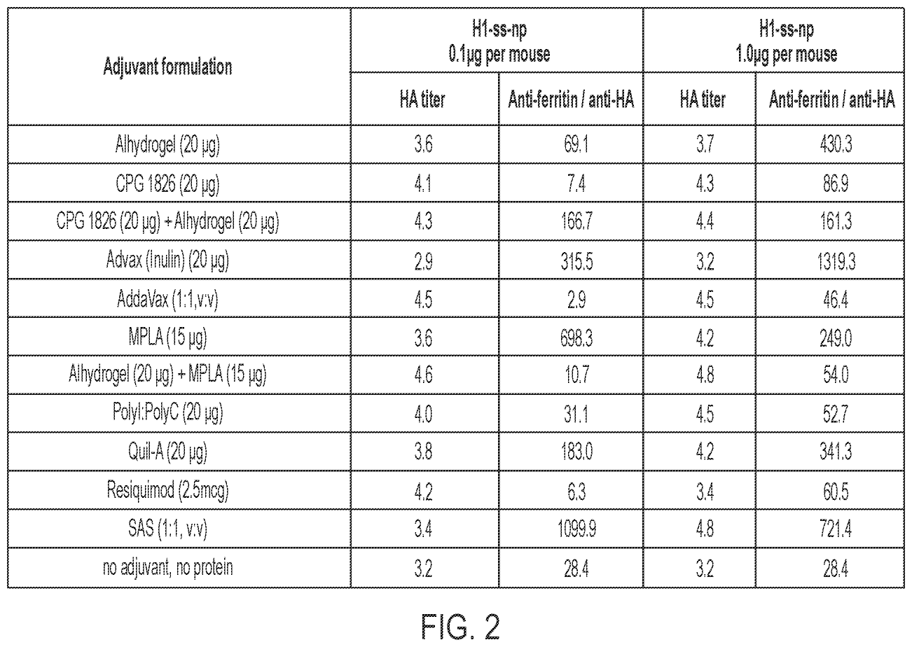

Results are summarized in FIG. 1, showing the percent survival of mice injected with each of the vaccine formulations. In general, mice injected with 1 μg dose of H1-ss-np had higher survival rates than those injected with 0.1 μg dose of H1-ss-np. FIG. 2 summarizes the relationship of IgG anti-ferritin serum titers to IgG anti-HA serum titers. Low survival and low antibody titers were found for Alhydrogel as a single adjuvant. Vaccine formulations associated with 80% or greater survival were AddaVax emulsion, Alhydrogel combined with MPLA, Alhydrogel combined with CPG1826, or Quil-A. Of these, AddaVax also displayed highest weight recovery. CPG1826, AddaVax, and PolyI:PolyC had lowest anti-ferritin/anti-HA values.

### Example 2. Assessment of H-ss-np with Adjuvant Combinations

Formulations of H-ss-np and various adjuvants were assessed for protective immunity in mice. Experiments were conducted as described in Example 1, except the adjuvants tested were QS-21, CpG 1826, Monophosophoryl Lipid (MPL), the Sigma Adjuvant System (SAS), AddaVax, and Al(OH)₃ (aluminum hydroxide). Experimental design is shown in Table 2.

TABLE 2
Example 2 Experiment Design

| | Antigen | | Adjuvant | | Matrix | | Schedule | | | No. |
|---|---|---|---|---|---|---|---|---|---|---|
| | | Dose | | Dose | | Dose | Vaccination | Challenge | Bleed | of |
| Grp | Ag | (μg) | Adjuvant | (μg) | Matrix | (μg) | (Days) | (Day) | (Days) | Mice |
| 1 | H1- | 0.1 | QS-21 | 20 | None | — | 0, 21 | 49 | 34-35, | 10 |
| 2 | ss-np | 0.1 | | 5 | | — | | | 47-48 | 10 |
| 3 | | 1 | | 20 | | — | | | | 10 |
| 4 | | 1 | | 5 | | — | | | | 10 |
| 5 | | 0.1 | | 20 | AddaVax | 2.5 | | | | 10 |
| 6 | | 0.1 | | 5 | | 2.5 | | | | 10 |
| 7 | | 1 | | 20 | | 2.5 | | | | 10 |
| 8 | | 1 | | 5 | | 2.5 | | | | 10 |
| 9 | | 0.1 | CPG1826 | 30 | None | — | | | | 10 |
| 10 | | 0.1 | | 5 | | — | | | | 10 |
| 11 | | 1 | | 30 | | — | | | | 10 |
| 12 | | 1 | | 5 | | — | | | | 10 |
| 13 | | 0.1 | | 30 | Al(OH)₃ | 30* | | | | 10 |
| 14 | | 0.1 | | 5 | | 5* | | | | 10 |
| 15 | | 1 | | 30 | | 30* | | | | 10 |
| 16 | | 1 | | 5 | | 5* | | | | 10 |
| 17 | | 0.1 | MPL | 10 | None | — | | | | 10 |
| 18 | | 0.1 | | 2.5 | | — | | | | 10 |
| 19 | | 1 | | 10 | | — | | | | 10 |
| 20 | | 1 | | 2.5 | | — | | | | 10 |
| 21 | | 0.1 | | 10 | Al(OH)₃ | 10* | | | | 10 |
| 22 | | 0.1 | | 2.5 | | 2.5* | | | | 10 |
| 23 | | 1 | | 10 | | 10* | | | | 10 |
| 24 | | 1 | | 2.5 | | 2.5* | | | | 10 |
| 25 | | 0.1 | None | — | None | — | | | | 10 |
| 26 | | 1 | | — | | — | | | | 10 |
| 27 | | 0.1 | SAS | — | | — | | | | 10 |
| 28 | | 1 | | — | | — | | | | 10 |
| 29 | | 0 | None | — | — | — | | | | 10 |

*Amount of aluminum (μg)

### Results

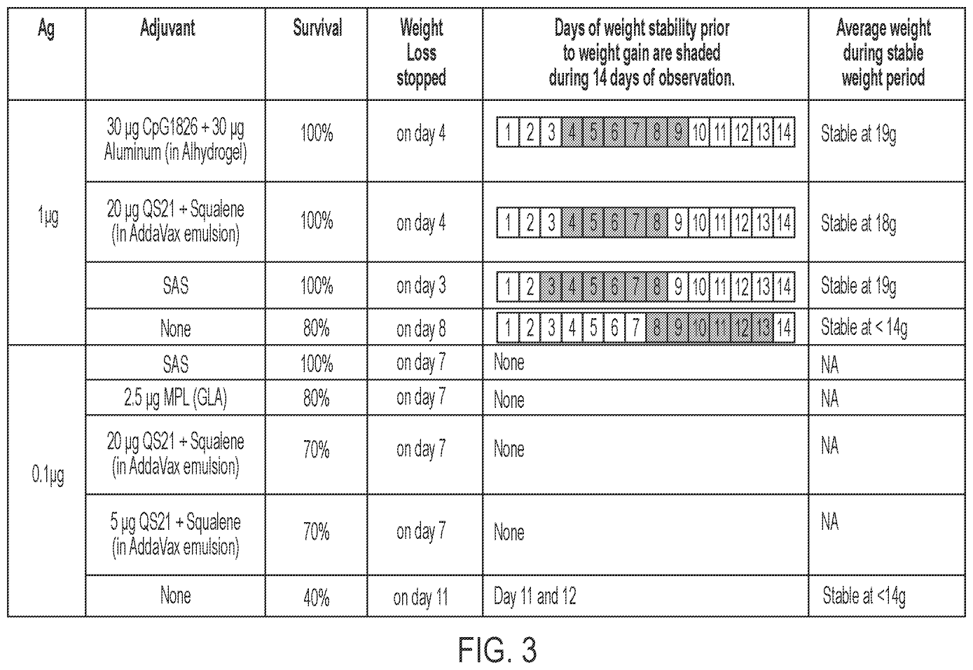

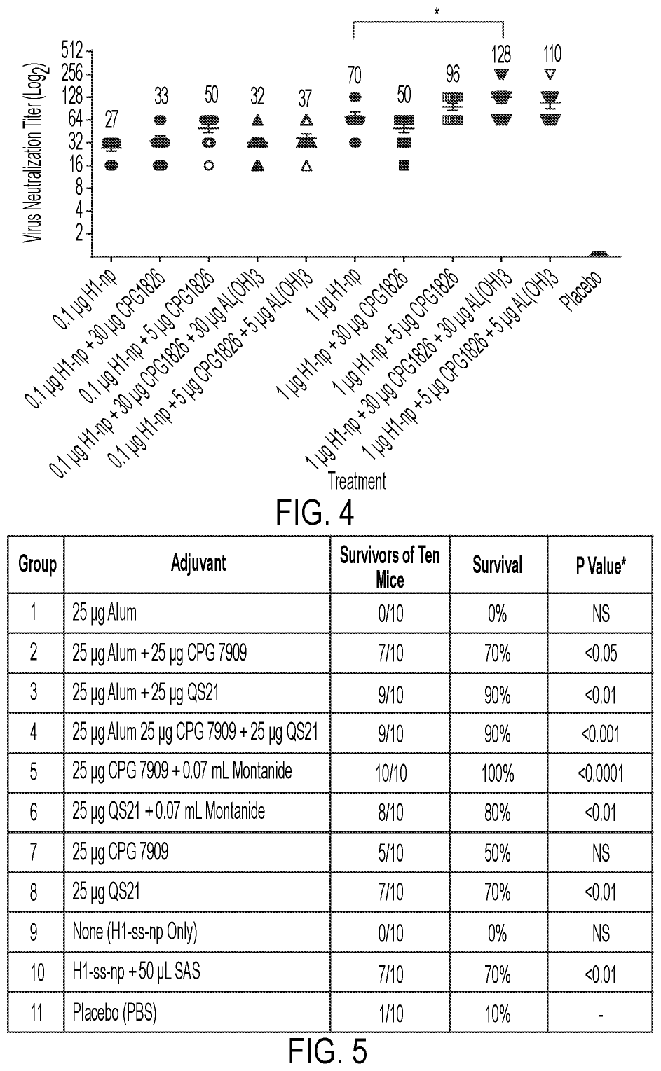

Results are summarized in Table 3 and FIGS. 3 and 4. Statistically significant survival benefits were observed with the QS-21 adjuvant but only when used in combination with the AddaVax matrix. The CpG1826 adjuvant provided statistically significant protection from both mortality and weight-loss but the effect was more pronounced in mice where CpG was used in combination with Al(OH)₃.

FIG. 4 shows the virus neutralization titers for each member of the relevant groups with the mean titer for each group displayed above the data points. As shown in FIG. 4, among the relevant groups, Group 15 [1 μg H1-ss-np+30 μg CPG1826+30 μg Al(OH)₃] and Group 16 [1 μg H1-ss-np+5 μg CPG1826+5 μg Al(OH)₃] had the highest Day 47 mean neutralization titers, 128 and 110, respectively and were the only relevant groups with some individuals achieving a titer of 256. Groups containing H1-ss-np alone or in combination with CPG1826 without Alhydrogel all had titers of 128 or less. Group 15, 1 μg H1-np+30 μg CPG1826+5 μg Al(OH)₃, had a statistically significant higher neutralization titer compared to group 26, 1 μg H1-ss-np alone (p<0.02). While there was no statistical significance between the neutralization titer of group 15 and group 12, 1 μg H1-ss-np+5 μg CPG1826, the mean titer of group 15 was higher at 128, compared to group 12 with a mean titer of 96.

TABLE 3
Survival Rates

| | | No. of Survivors/ | | P Value |
|---|---|---|---|---|
| | | Total Number of | Percent | (compared to placebo- |
| G | Vaccine/Adjuvant/Matrix | Mice | Survival | treated mice) |
| 1 | 0.1 μg H1-ss-np + 20 μg QS-21 | 2/10 | 20% | NS |
| 2 | 0.1 μg H1-ss-np + 5 μg QS-21 | 3/10 | 30% | NS |
| 3 | 1 μg H1-ss-np + 20 μg QS-21 | 1/10 | 10% | NS |
| 4 | 1 μg H1-ss-np + 5 μg QS-21 | 1/10 | 10% | NS |
| 5 | 0.1 μg H1-ss-np + 20 μg QS-21 + | 7/10 | 70% | NS |
| | 2.5 μg Adda Vax | | | |
| 6 | 0.1 μg H1-ss-np + 5 μg QS-21 + | 7/10 | 70% | <0.05 |
| | 2.5 μg Adda Vax | | | |
| 7 | 1 μg H1-ss-np + 20 μg QS-21 + 2.5 | 10/10 | 100% | <0.001 |
| | μg Adda Vax | | | |
| 8 | 1 μg H1-ss-np + 5 μg QS-21 + 2.5 | 8/10 | 80% | <0.01 |
| | μg Adda Vax | | | |
| 9 | 0.1 μg H1-ss-np + 30 μg CPG1826 | 4/10 | 40% | NS |
| 10 | 0.1 μg H1-ss-np + 5 μg CPG1826 | 6/10 | 60% | <0.05 |
| 11 | 1 μg H1-ss-np + 30 μg CPG1826 | 8/10 | 80% | <0.05 |
| 12 | 1 μg H1-ss-np + 5 μg CPG1826 | 9/10 | 90% | <0.001 |
| 13 | 0.1 μg H1-ss-np + 30 μg CPG1826 + | 6/10 | 60% | NS |
| | 30 μg Al(OH)₃ | | | |
| 14 | 0.1 μg H1-ss-np + 5 μg CPG1826 + | 4/10 | 40% | <0.05 |
| | 5 μg Al(OH)₃ | | | |
| 15 | 1 μg H1-ss-np + 30 μg CPG1826 + | 10/10 | 100% | <0.001 |
| | 30 μg Al(OH)₃ | | | |
| 16 | 1 μg H1-ss-np + 5 μg CPG1826 + 5 | 10/10 | 100% | <0.001 |
| | μg Al(OH)₃ | | | |
| 17 | 0.1 μg H1-ss-np + 10 μg MPL | 4/10 | 40% | NS |
| 18 | 0.1 μg H1-ss-np + 2.5 μg MPL | 7/10 | 70% | <0.05 |
| 19 | 1 μg H1-ss-np + 10 μg MPL | 7/10 | 70% | <0.05 |
| 20 | 1 μg H1-ss-np + 2.5 μg MPL | 10/10 | 100% | <0.001 |
| 21 | 0.1 μg H1-ss-np + 10 μg MPL + 10 | 5/10 | 50% | NS |
| | μg Al(OH)₃ | | | |
| 22 | 0.1 μg H1-ss-np + 2.5 μg MPL + 10 | 3/10 | 30% | NS |
| | μg Al(OH)₃ | | | |
| 23 | 1 μg H1-ss-np + 10 μg MPL + 10 | 7/10 | 70% | <0.05 |
| | μg Al(OH)₃ | | | |
| 24 | 1 μg H1-ss-np + 2.5 μg MPL + 10 | 9/10 | 90% | <0.01 |
| | μg Al(OH)₃ | | | |
| 25 | 0.1 μg H1-ss-np | 4/10 | 40% | NS |
| 26 | 1 μg H1-ss-np | 8/10 | 80% | <0.01 |
| 27 | 0.1 μg H1-ss-np + SAS | 10/10 | 100% | <0.001 |
| 28 | 1 μg H1-ss-np + SAS | 10/10 | 100% | <0.001 |
| 29 | None | 2/10 | 20% | — |

### Example 3. Assessment of H1-Ss-Np with Adjuvant Combinations

Formulations of H1-ss-np and various adjuvants were assessed for protective immunity in mice. Experiments were conducted as described in Example 1, except the adjuvants tested were those shown in Table 4.

TABLE 4
Example 3 Experiment Design

| | | H1-ss-np | Vaccination | Bleed | Challenge | No. of |
|---|---|---|---|---|---|---|
| Grp | Adjuvant | Dose (μg) | Days | Days | Virus | Mice |
| 1 | 25 μg Alum | 1 | 0 and 21 | 21 | A/CA/04/2009 | 10 |
| 2 | 25 μg Alum + | 1 | | | (H1N1) | 10 |
| | 25 μg CPG 7909 | | | | | |
| 3 | 25 μg Alum + | 1 | | | | 10 |
| | 25 μg QS21 | | | | | |
| 4 | 25 μg Alum + | 1 | | | | 10 |
| | 25 μg CPG 7909 + | | | | | |
| | 25 μg QS21 | | | | | |
| 5 | 25 μg CPG 7909 + | 1 | | | | 10 |
| | 0.07 mL Montanide | | | | | |
| 6 | 25 μg QS21 + | 1 | | | | 10 |
| | 0.07 mL Montanide | | | | | |
| 7 | 25 μg CPG 7909 | 1 | | | | 10 |
| 8 | 25 μg QS21 | 1 | | | | 10 |
| 9 | None (H1-ss-np Only) | 1 | | | | 10 |
| 10 | 50 μL SAS | 1 | | | | 10 |
| 11 | Placebo (PBS) | 0 | | | | 10 |

### Results

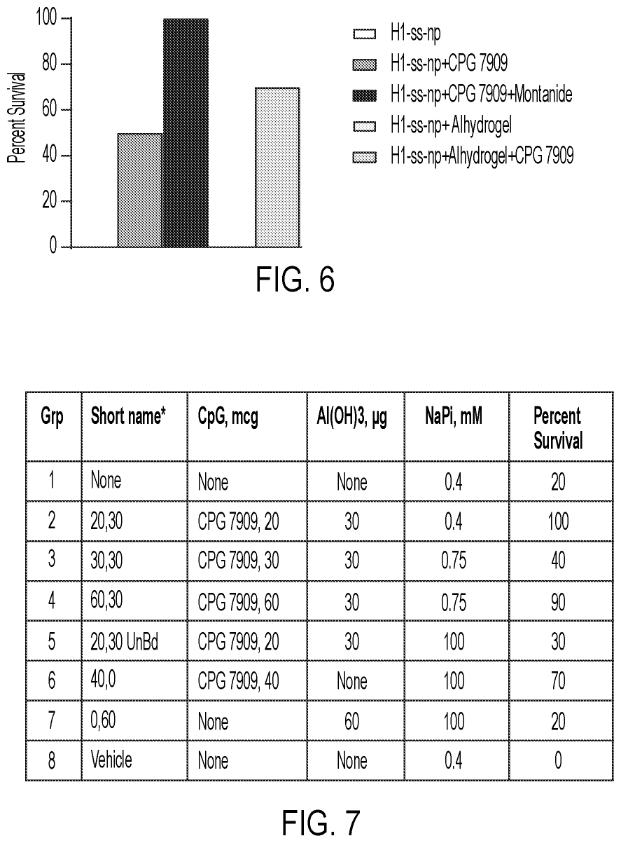

Results are shown in FIG. 5. The H1-ss-np vaccine candidate provided statistically significant (when compared with placebo) protection from mortality (700%) following influenza A (H1N1pdm) virus challenge when combined with CPG 7909 and Alhydrogel adjuvants or Sigma Adjuvant System (SAS). H1-ss-np adjuvanted with CPG 7909 adjuvant alone (Group 3) provided 50% survival, which was not statistically significant compared with placebo. H1-ss-np adjuvanted with Alhydrogel (Group 1) or unadjuvanted H1-ss-np (Group 5) provided no protection from mortality. FIG. 6 shows a plot summarizing the results in FIG. 5.

The study demonstrated that H1-ss-np vaccine candidate could provide statistically significant protection from mortality in mice following challenge with influenza A (H1N1pdm) virus when combined with CPG 7909 and Alhydrogel adjuvants. Neither H1+either adjuvant on its own nor unadjuvanted H1-ss-np could provide statistically significant protection from mortality.

### Example 4. Assessment of Alhydrogel+CpG ODN Formulations

Formulations of H1-ss-np and various adjuvants were assessed for protective immunity in mice. Experiments were conducted as described in Example 1, except the adjuvants tested were combinations of Alhydrogel and CpG ODN, with varying concentrations of phosphate, as shown in Table 5. The antigen (H1-ss-np) was added last, after the CPG 7909 was allowed to adsorb to Alhydrogel. Final phosphate concentrations of 0.4 or 0.75 mM allowed for CPG 7909 binding/adsorption to Alhydrogel while blocking antigen (H1-ss-np) binding/adsorption to Alhydrogel. 100 mM phosphate blocked binding/adsorption of both CPG 7909 and antigen to Alhydrogel (data not shown).

TABLE 5
Example 4 Experiment Design

| | | H1-ss-np | NaPi, | Vaccination | Bleed | Challenge | No. of |
|---|---|---|---|---|---|---|---|
| Grp | Adjuvant | Dose (μg) | mM | Days | Days | Virus | Mice |
| 1 | None | 1 | 0.4 | 0 and 21 | 0, 21, | A/CA/04/2009 | 10 |
| 2 | 20 μg CPG 7909 + | 1 | 0.4 | | 35, | (H1N1) | 10 |
| | 30 μg Alhydrogel | | | | and | | |
| 3 | 30 μg CPG 7909 + | 1 | 0.75 | | 63 | | 10 |
| | 30 μg Alhydrogel | | | | | | |
| 4 | 60 μg CPG 7909 + | 1 | 0.75 | | | | 10 |
| | 30 μg Alhydrogel | | | | | | |
| 5 | 20 μg CPG 7909 + | 1 | 100 | | | | 10 |
| | 30 μg Alhydrogel | | | | | | |
| | (Unbound) | | | | | | |
| 6 | 40 μg CPG 7909 | 1 | 100 | | | | 10 |
| 7 | 60 μg Alhydrogel | 1 | 100 | | | | 10 |
| 8 | Vehicle | 0 | 0.4 | | | | 10 |

### Results

The survival results are summarized in FIG. 7. When combined with CPG 7909+Alhydrogel adjuvants, H1-ss-np provided 90% (Group 4) or 100% (Group 2) protection from mortality in mice following challenge with influenza A (H1N1pdm) virus. Both these vaccine candidates provided statistically significant protection from mortality when compared with groups which received vehicle (Group 15), H1-ss-np alone (Group 1), or H1-ss-np+Alhydrogel (Group 14).

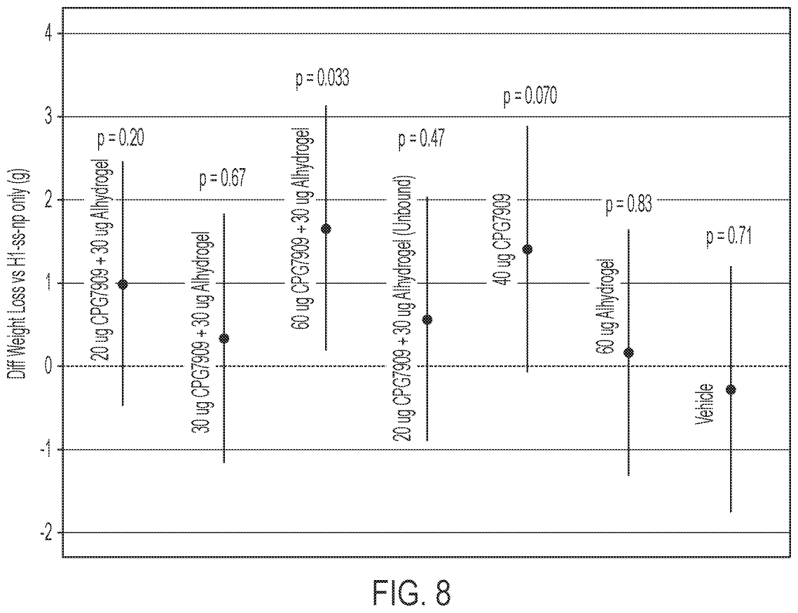

FIG. 8 shows the relative difference in weight loss between Day 0 and the average of Days 4-6 post-challenge (when maximum weight loss occurred) of selected groups when compared with Group 1, H1-ss-np alone. H1-ss-np combined with Alhydrogel, CPG 7909 or each combination of CPG 7909+Alhydrogel as adjuvants provided more protection from weight loss than H1-ss-np alone. Each point represents the difference in weight loss from the treatment group vs the H1-ss-np only group, with the line representing the 95% confidence interval. When the entire line is above 0 (horizontal line in the middle of the graph), this indicates less weight loss in that group than in the H1-ss-np only group. Of these groups, only H1-ss-np+60 μg CPG 7909+30 μg aluminum as Alhydrogel provided significantly (p<0.05) more protection from weight loss compared with H1-ss-np alone.

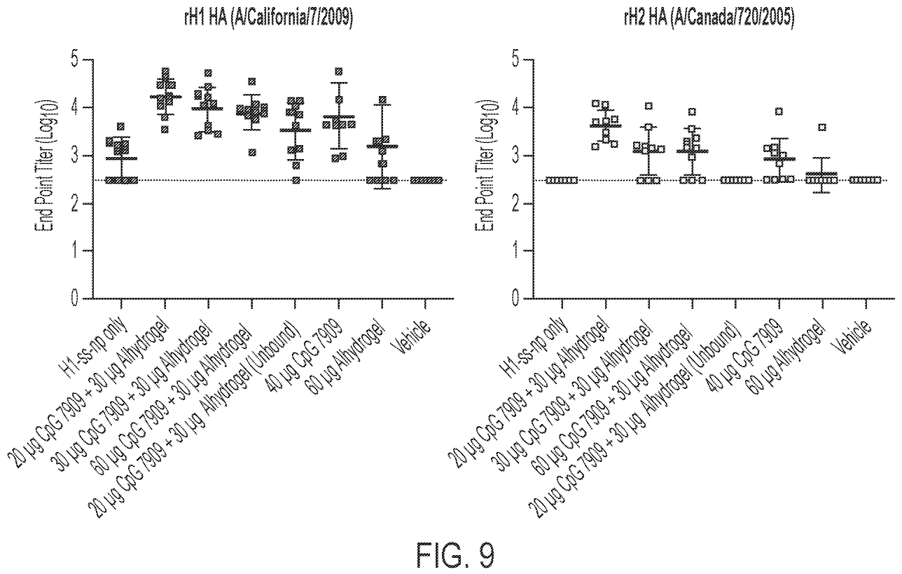

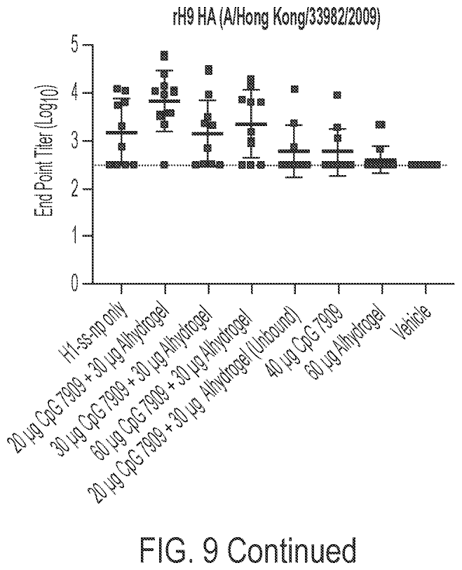

Immune responses were measured by direct binding ELISA and endpoint titers were calculated. Groups that were immunized with H1-ss-np adjuvanted with CPG 7909 bound to Alhydrogel had higher (statistically significant) endpoint rH1, rH2 and rH9 anti-HA titers than groups that were adjuvanted with unbound CPG 7909+Alhydrogel, Alhydrogel alone, unadjuvanted, or vehicle alone (FIG. 9).

This study demonstrated that H1-ss-np, when combined with 60 μg CPG 7909+30 μg aluminum as Alhydrogel, induced higher (statistically significant) rH1, rH2 and rH9 endpoint titers and provided statistically significant protection from mortality and weight loss after challenge with influenza A (H1N1pdm) virus when compared with H1-ss-np alone or adjuvanted with Alhydrogel.

### Example 5. Assessment of Alhydrogel+CpG ODN Formulations

Formulations of H1-ss-np and various adjuvants were assessed for protective immunity in mice. Experiments were conducted as described in Example 1, except the adjuvants tested were combinations of Alhydrogel (“Alum”) and CpG ODN, as shown in Table 6. H1-ss-np in 0.5 mM Tris and 0.9% sodium chloride was diluted approximately 14-fold further in saline to achieve a final buffer of 0.04% Tris and 0.9% sodium chloride. No additional phosphate was added. H1-ss-np in this buffer was admixed with the indicated amounts of CpG ODN and Alum in table 6.

TABLE 6
Example 5 Experiment Design

| | | H1-ss-np | Vaccination | Bleed | Challenge | No. of |
|---|---|---|---|---|---|---|
| Grp | Adjuvant | Dose (μg) | Days | Days | Virus | Mice |
| 1 | CPG 7909 20 μg | 1 | 0 and 21 | 0, 21, | A/CA/04/2009 | 14 |
| 2 | CPG 7909 20 μg + | 1 | | 35, | (H1N1) | 14 |
| | Alum 30 μg | | | and | | |
| 3 | CPG 7909 20 μg + | 1 | | 63 | | 14 |
| | Alum 40 μg + | | | | | |
| | QS-21 20 μg | | | | | |
| 4 | CPG 7909 20 μg + | 1 | | | | 14 |
| | QS-21 20 μg | | | | | |
| 5 | CPG 7909 60 μg + | 1 | | | | 14 |
| | Alum 30 μg | | | | | |
| 6 | QS-21 20 μg + | 1 | | | | 14 |
| | NE01 5% | | | | | |
| 7 | QS-21 20 μg + | 1 | | | | 14 |
| | NE02 20% | | | | | |
| 8 | QS-21 20 μg + | 1 | | | | 14 |
| | NE01 5% + | | | | | |
| | GLA 5 ug | | | | | |
| 9 | QS-21 20 μg + | 1 | | | | 14 |
| | NE02 20% + | | | | | |
| | GLA 5 μg | | | | | |
| 10 | Alum 30 μg | 1 | | | | 14 |
| 11 | NE01 5% | 1 | | | | 14 |
| 12 | NE02 20% | 1 | | | | 14 |
| 13 | Placebo | None | | | | 14 |
| 14 | SAS + H1-ss-np | None | | | | 10 |
| | (1 μg) | | | | | |

### Results

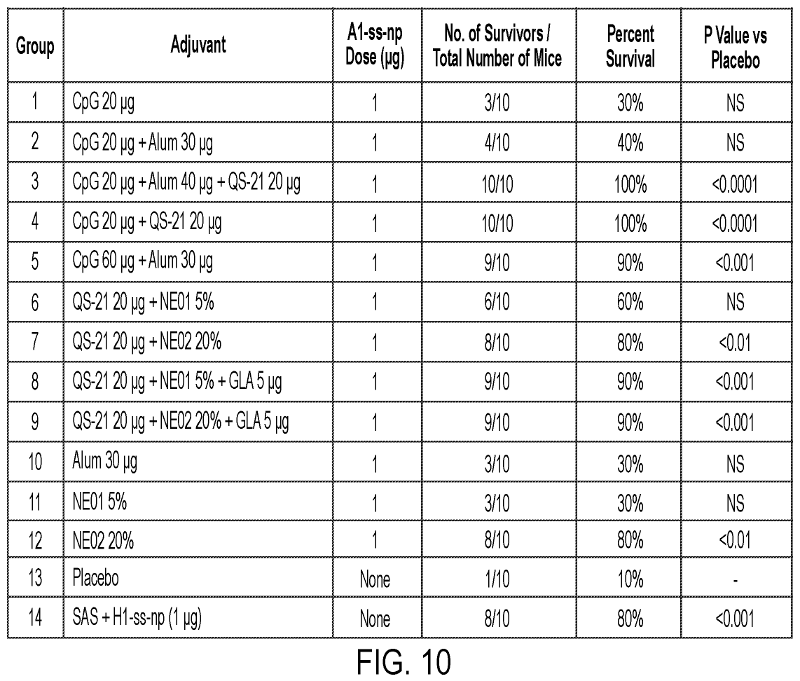

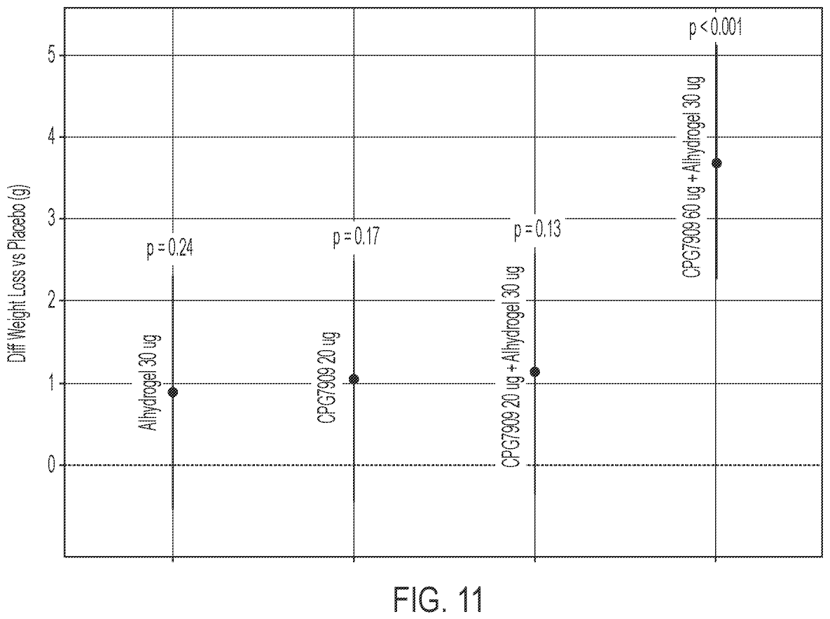

The survival results of relevant groups are summarized in FIG. 10. The study demonstrated that the H1-ss-np+60 μg CPG 7909+30 μg aluminum as Alhydrogel vaccine candidate (Group 3) provided statistically significant protection from mortality following challenge with influenza A (H1N1pdm) virus when compared to placebo (Group 5), while H1-ss-np+20 μg CPG 7909+30 μg Alhydrogel (Group 2) did not. FIG. 11 shows the relative weight loss between Day 0 and the average of Days 5-7 post-challenge (when maximum weight loss occurred) of Groups 1-4 compared with placebo. Each point represents the difference in weight loss from the treatment group vs the placebo group, with the line representing the 95% confidence interval. When the entire line is above 0 (horizontal line in the middle of the graph), this indicates less weight loss in that group than in the placebo group. Group 3 also was significantly (p<0.0001) protected from weight loss compared to placebo, while Group 2 was not. H1-ss-np combined with 20 μg CPG 7909 (Group 1) or 30 μg aluminum as Alhydrogel (Group 10) alone did not provide statistically significant protection from mortality or weight loss.

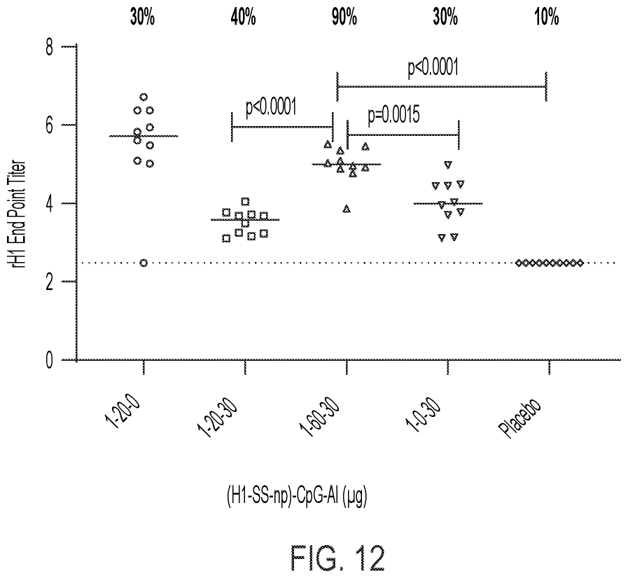

ELISA end point titer analysis results are summarized in FIG. 12. End point titers against rH1 are significantly higher in Group 5 (H1-ss-np+60 μg CPG 7909+30 μg Alhydrogel) than Group 2 (H1-ss-np+20 μg CPG 7909+30 μg Alhydrogel) (p<0.0001), Group 10 (H1-ss-np+30 μg Alhydrogel) (p=0.0015) or Group 13 (placebo) (p<0.0001).

This study demonstrated that the H1-ss-np+60 μg CPG 7909+30 μg aluminum as Alhydrogel vaccine candidate provided protection in mice from mortality and weight loss following challenge with influenza A (H1N1pdm) virus. This combination of adjuvants also induced statistically significant anti-H1 endpoint titers. Despite the fact that H1-ss-np+CPG 7909 protected mice from mortality in Example 4, neither adjuvant by itself was able to provide protection in this study when mixed with H1-ss-np, demonstrating that both CPG 7909 and Alhydrogel contribute to the protection. The 60 μg/30 μg CPG 7909/aluminum ratio, which is similar to the 500 μg/300 μg ratio in the candidate bivalent vaccine, provided better protection from mortality and weight loss after challenge in this study than the 20 μg/30 μg CPG 7909/aluminum ratio.

### Example 6. Assessment of Bivalent Antigen Formulation with Alhydrogel and CpG ODN

The efficacy in BALB/c mice of two bivalent vaccines comprised of H1-ss-np+H10-ss-np+different amounts of CPG 7909 and Alhydrogel adjuvants was evaluated in two studies. Each study had separate arms that were challenged with influenza A/California/04/09 (H1N1pdm) or influenza A/Victoria/3/75 (H3N2) virus. Study 1 evaluated dilutions of a proposed human dose of 20 μg each H1 and H10-ss-np+400 μg CPG 7909+600 μg aluminum as Alhydrogel, while study 2 evaluated dilutions of a proposed human dose of 20 μg each H1 and H10-ss-np+500 μg CPG 7909+300 μg aluminum as Alhydrogel. All vaccines were formulated as described in Example 5 and contained a final concentration of 0.4 mM phosphate to allow for maximal CPG 7909 binding/adsorption to Alhydrogel and no binding/adsorption of antigen to Alhydrogel. The antigen (H1-ss-np) was added last, after the CPG 7909 was allowed to adsorb to Alhydrogel. In addition, bivalent vaccines containing Alhydrogel and CPG 7909 were evaluated against both influenza A/California/04/09 (H1N1pdm) and influenza A/Victoria/3/75 (H3N2) virus challenge. Monovalent vaccines were also evaluated, with monovalent H1-ss-np vaccines evaluated against influenza A/California/04/09 (H1N1pdm) virus challenge and monovalent H10-ss-np vaccines evaluated against influenza A/Victoria/3/75 (H3N2) virus challenge. 26 groups of 10 female BALB/c mice, 6-8 weeks old, were immunized twice IM, three weeks apart, and challenged IN with 1×10⁵ (3×LD₅₀) CCID₅₀ of the mouse-adapted challenge strain four weeks after the last immunization. Mice were observed for morbidity and mortality for 14 days after challenge.

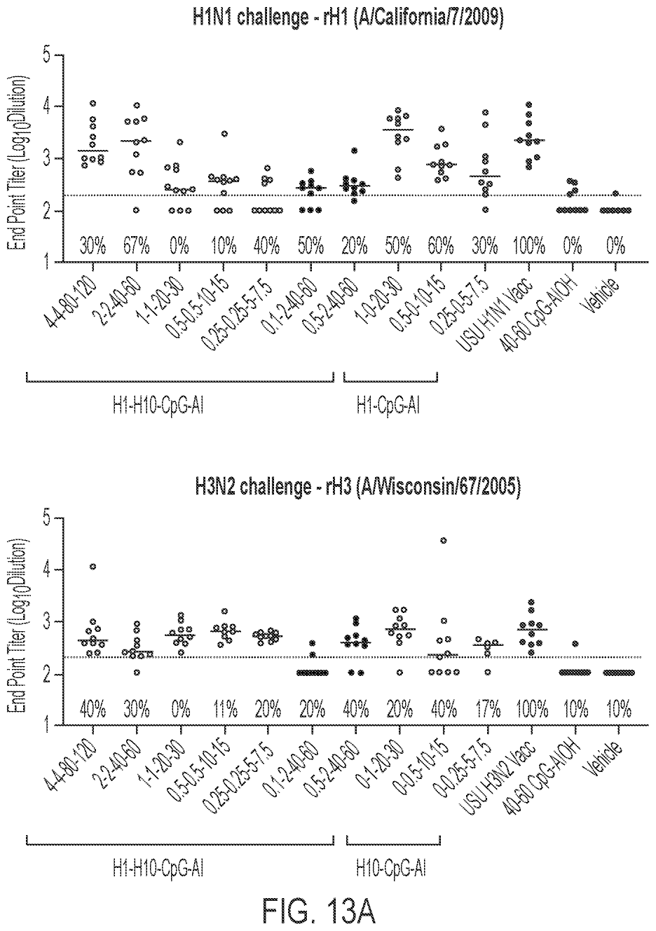

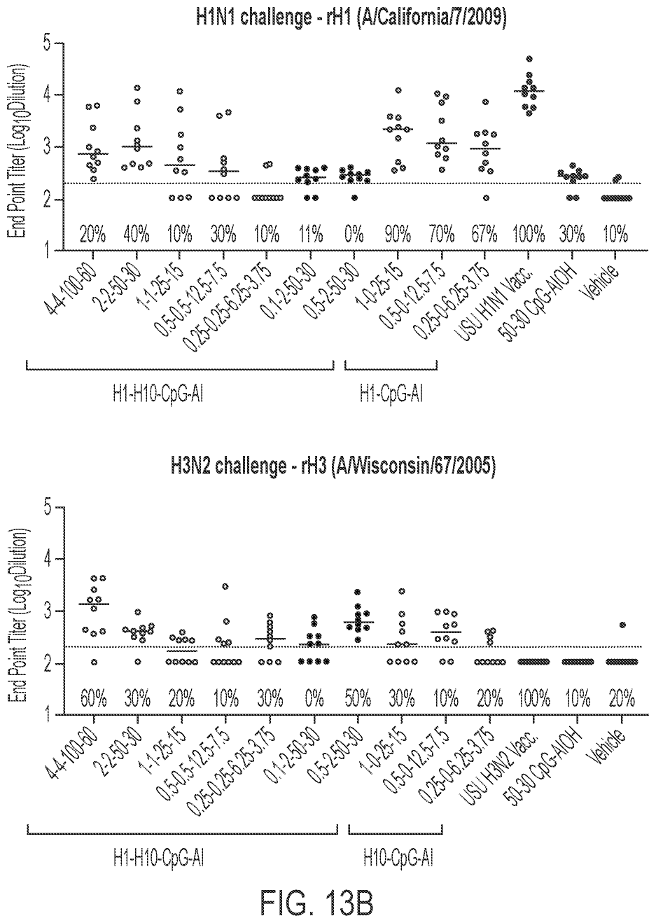

In Study 1, one bivalent vaccine dilution comprised of a final mouse dose of 2 μg each H1 and H10-ss-np+40 μg CPG 7909+60 μg aluminum provided statistically significant protection from mortality (p<0.01) and weight loss (p<0.05) following influenza A (H1N1pdm) virus challenge compared with placebo. One monovalent H1-ss-np vaccine comprised of 1 μg H1-ss-np+20 μg CPG 7909+30 μg aluminum provided statistically significant protection from mortality (p<0.05) and weight loss (p<0.05) and another comprised of 0.5 μg H1-ss-np+10 μg CPG 7909+15 μg aluminum provided statistically significant protection from mortality (p<0.01) following influenza A (H1N1pdm) virus challenge compared with placebo. Results for percent survival are shown in FIG. 13A (percentages above x-axis).

In Study 2, three monovalent vaccine dilutions comprised of 1 μg H1-ss-np+25 μg CPG 7909+15 μg aluminum (p<0.001), 0.5 μg H1-ss-np+12.5 μg CPG 7909+7.5 μg aluminum (p<0.05), or 0.25 μg H1-ss-np+6.25 μg CPG7909+3.75 μg aluminum (p<0.05) provided statistically significant protection from mortality following influenza A (H1N1pdm) virus challenge. One bivalent vaccine dilution, comprised of 4 μg each of H1-ss-np and H10-ss-np+100 μg CPG 7909+60 μg Alhydrogel provided 60% survival, an improvement over placebo (20% survival) following influenza A (H3N2) virus challenge. Results for percent survival are shown in FIG. 13B (percentages above x-axis).

Mice immunized with bivalent vaccines in either Study 1 or 2 generated immune responses against rH1, rH3, rH5, rH7, rH9 and rH10 HA as measured by ELISA. FIGS. 13A and 13B show group survival and endpoint titer responses against rH1 and rH3 in Study 1 (FIG. 13A) and Study 2 (FIG. 13B).

Study 1 demonstrated that one bivalent vaccine formulation and two monovalent vaccine formulations provided statistically significant protection from mortality following influenza A (H1N1pdm) virus challenge. In Study 2, three monovalent vaccine formulations provided statistically significant protection from mortality following influenza A (H1N1pdm) virus challenge. No vaccine formulations in either study provided statistically significant protection against mortality following influenza A (H3N2) virus challenge. The highest survival provided against influenza A (H3N2) virus challenge was 60% in the group in Study 2 which was administered a formulation of 4 μg H1-ss-np+4 μg H10-ss-np+100 μg CPG 7909+60 μg aluminum as Alhydrogel. On the basis of the protection provided by the three monovalent H1 vaccines in Study 2, and the fact that only the formulation with 60 μg CPG 7909+30 μg aluminum as Alhydrogel provided protection from mortality and weight loss in both Example 4 and Example 5, the human adjuvant dose mass ratio of 500 μg CPG 7909 plus 300 μg aluminum as Alhydrogel (or animal equivalent dose) was selected for future studies.

### Example 7. Assessment of H10-Ss-Np with Alhydrogel and CpG ODN

Three different lots of H10-ss-np, when formulated with CPG 7909+Alhydrogel, were evaluated for their ability to protect mice from mortality and weight loss following challenge with influenza A (H3N2) virus. Two dose levels, 20 and 5 μg, of each lot were combined with 100 μg CPG 7909+60 μg aluminum as Alhydrogel. The dose levels were selected based on the results from Example 6. All vaccines were formulated as described in Example 5 and with a final phosphate concentration of 0.36 mM, allowing for binding/adsorption of CPG 7909 but not antigen to Alhydrogel. The antigen (H1-ss-np) was added last, after the CPG 7909 was allowed to adsorb to Alhydrogel. Seven groups of 10 female BALB/c mice were immunized IM three times, three weeks apart, and one group was injected with vehicle. Mice were challenged IN with 1×10₅(3×LD₅₀) CCID₅₀ mouse-adapted influenza A/Victoria/3/75 (H3N2) virus four weeks after the last immunization, and observed for 14 days for morbidity and mortality.

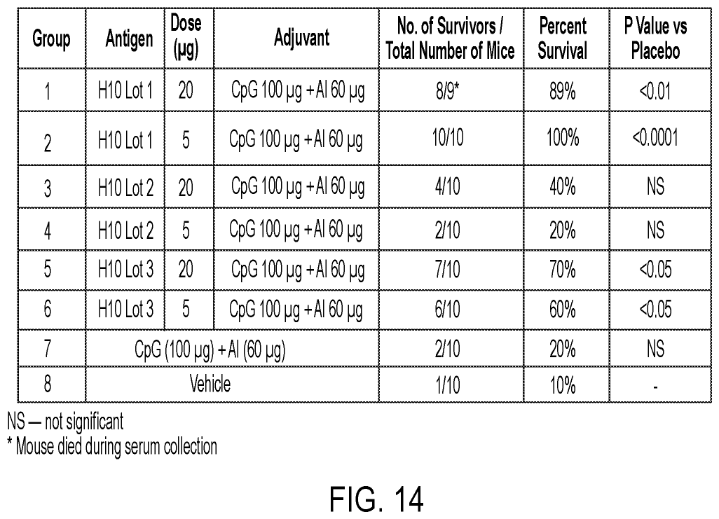

The survival results are summarized in FIG. 14. When combined with 100 μg CPG 7909+60 μg aluminum as Alhydrogel, Lots 1 and 3 of H10-ss-np provided statistically significant protection from mortality following influenza A (H3N2) virus challenge. Lot 3 of H10-SS-np also provided statistically significant protection from weight loss following influenza A (H3N2) virus challenge.

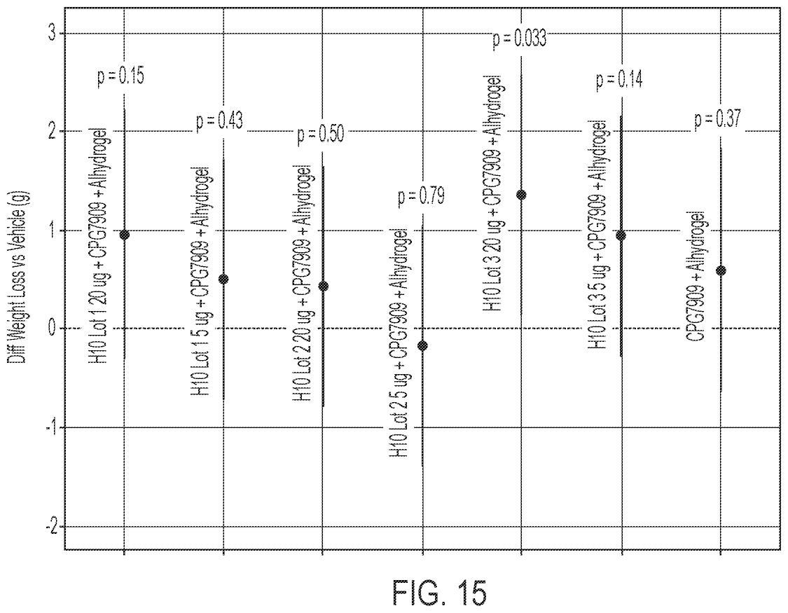

FIG. 15 shows the relative weight loss between Day 0 and the average of Days 5-7 (when maximum weight loss occurred). Each point represents the difference in weight loss from the treatment group vs the vehicle group, with the line representing the 95% confidence interval. When the entire line is above 0 (horizontal line in the middle of the graph), this indicates less weight loss in that group than in the vehicle group. Mice immunized with 20 μg of H10-ss-np Lot 3 combined with 100 μg CPG 7909+60 μg aluminum as Alhydrogel had significantly less weight loss, statistically, than mice immunized with vehicle.

This study demonstrated that H10-ss-np combined with 100 μg CPG 7909+60 μg aluminum as Alhydrogel can provide statistically significant protection from mortality and weight loss after challenge with influenza A (H3N2) virus.

### Example 8. Freeze-Thaw Testing

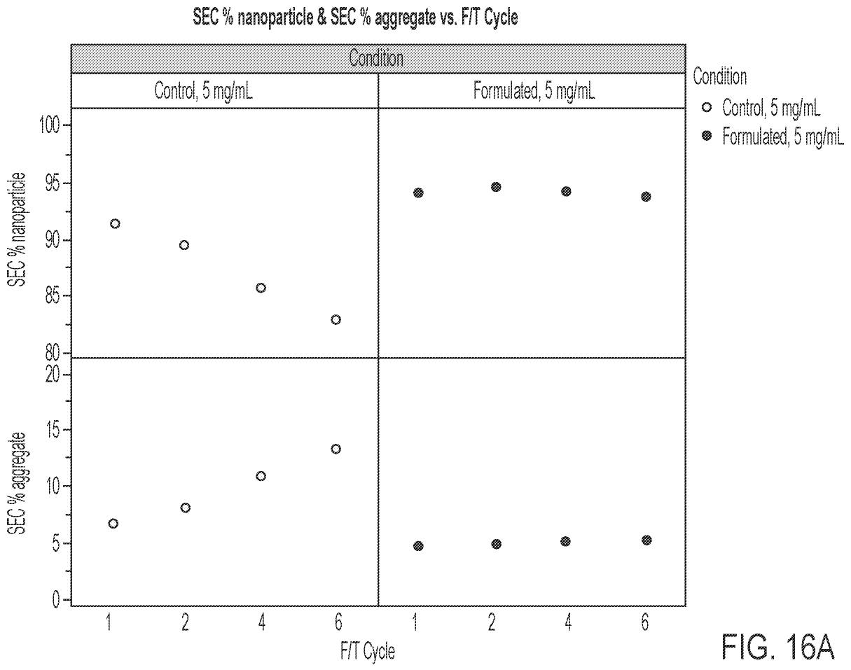

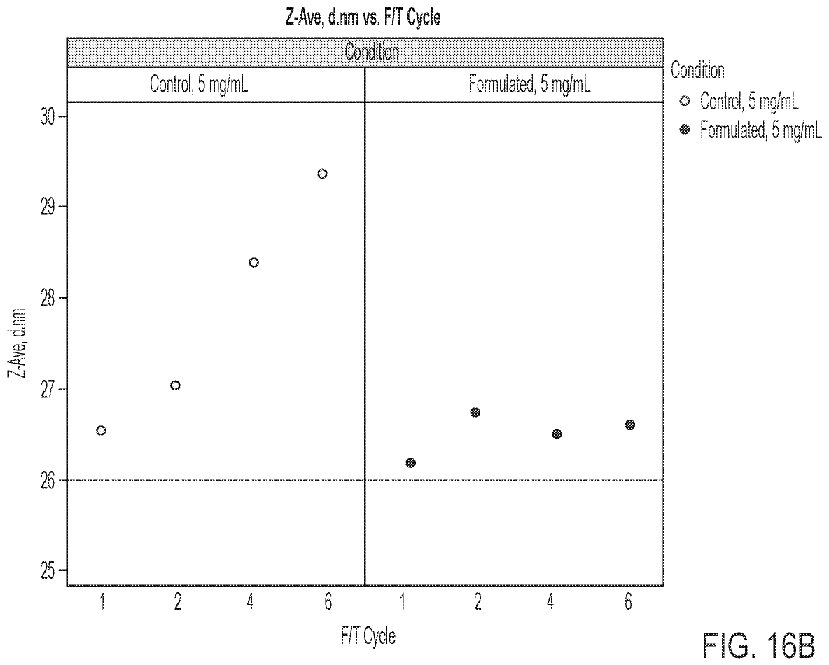

A freeze-thaw study was performed to test the effectiveness of cryoprotectant to prevent antigen aggregation after freeze-thaw. 5% sucrose was added to an H1-ss-np formulation, and 6 freeze-thaw cycles were performed. Antigen aggregation was analyzed by SEC-HPLC and DLS. Results are shown in FIG. 16A (SEC-HPLC) and 16B (DLS). The results show that in the control samples without sucrose, aggregation increased with the number of freeze-thaw cycles. Formulations with 5% sucrose did not show statistically significant increase in aggregation after 6 freeze-thaw cycles.

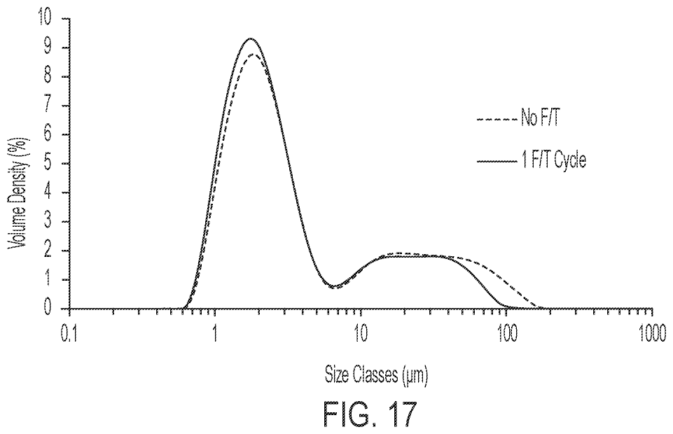

A further freeze-thaw study was performed to confirm that formulation Alhydrogel does not aggregate after freeze-thaw. The formulation shown in Table 7 was subjected to 0 or 1 freeze-thaw cycles. As shown in FIG. 17, particle size distribution of the Alhydrogel in the formulations did not change significantly, statistically, after 1 freeze-thaw cycle.

TABLE 7

| Component | [Final] | |
|---|---|---|
| Al(OH)₃ | 1.2 | mg/mL |
| WFI | NA | |
| Tris, pH 7.4 | 5 | mM |
| NaCl | 150 | mM |
| Tween 80 | 0.015% | |
| NaPi | 0.4 | mM |
| CpG | 0.8 | mg/mL |
| Sucrose | 20% | |
| H1 Antigen | 80 | μg/mL |

### Example 9. Assessment of Antigen and Adjuvant Binding

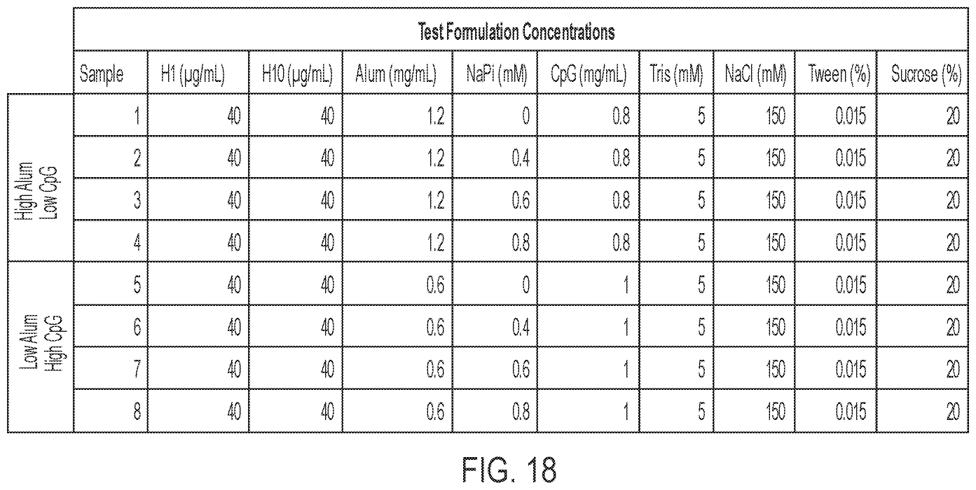

The binding of antigen and CpG 7909 ODN to Alhydrogel (“Alum”) at different sodium phosphate concentrations was determined. The test formulations are shown in FIG. 18.

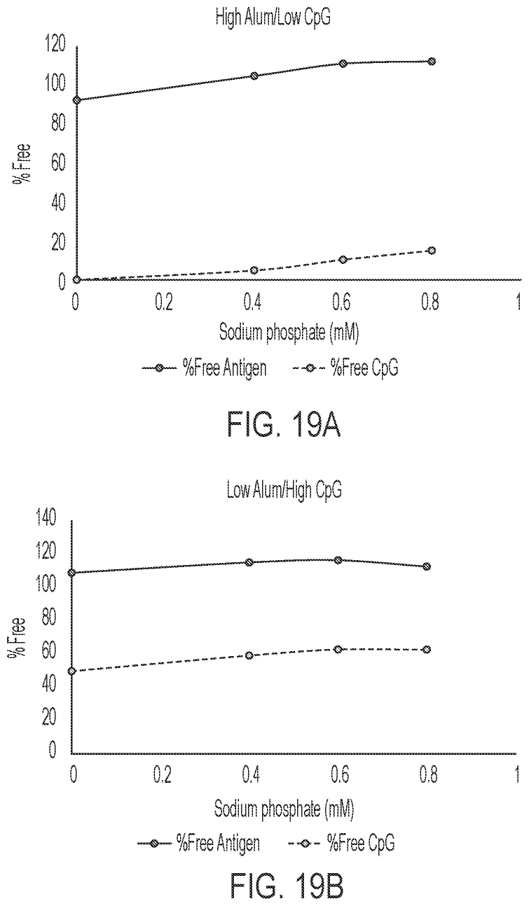

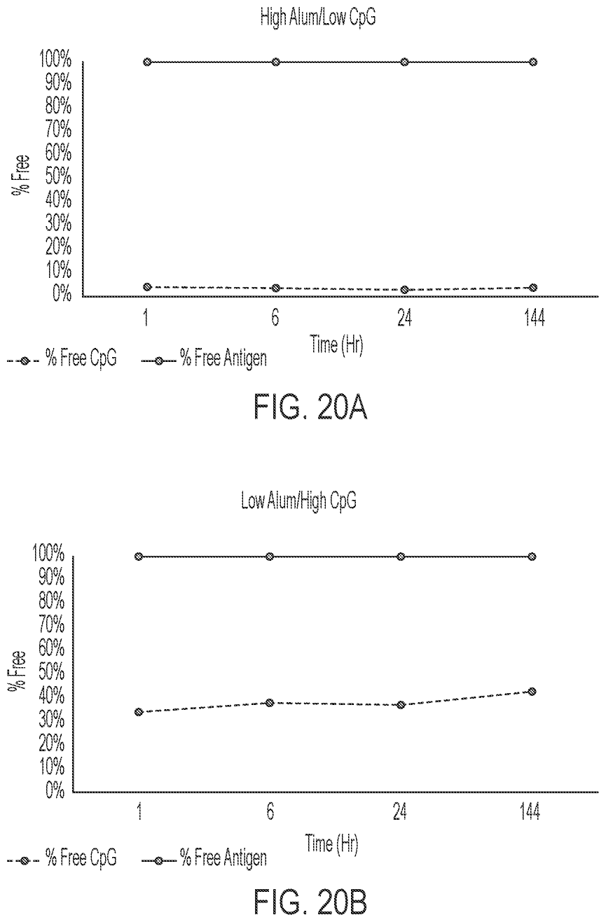

The binding results are shown in FIGS. 19A (high Alum/low CpG ODN, i.e., 1.2 mg/mL Alum and 0.8 mg/mL CpG ODN) and 19B (low Alum/high CpG ODN, i.e., 0.6 mg/mL Alum and 1.0 mg/mL CpG ODN). As shown in the results, the 0.4 mM sodium phosphate concentration kept CpG ODN bound and antigen in solution. FIGS. 20A (high Alum/low CpG ODN) and 20B (low Alum/high CpG ODN) show that the binding of antigen and CpG 7909 ODN in 0.4 mM phosphate was stable over a period of 6 days at 5° C.

### Example 10. Assessment of H1-Ss-Np with Alhydrogel+CpG ODN Formulations

Formulations of H1-ss-np and various adjuvants were assessed for protective immunity in mice. Experiments were conducted as described in Example 1, except the adjuvants tested were combinations of Alhydrogel and CpG ODN, as shown in Table 8. For groups 1 and 2, a microtube of protein antigen in saline was provided frozen and a microtube of adjuvant mixture in saline was provided at 4° C. For groups 5, 6, 7, 8, 9 and 10, vaccines were formulated similar to as described above with specific concentrations of phosphate thereby regulating the amount of antigen (nanoparticle) and CPG 7909 bound to Alhydrogel. The Unbound state was noted as (U). The bound state was noted as (B). The ratio indicates the amount (in μg) of H1-ss-np:Alhydrogel:CPG 7909 in the formulated vaccine dose.

TABLE 8
Example 10 Experiment Design

| | Vaccine | H1-ss-np | Vaccination | Bleed | Challenge | No. of |
|---|---|---|---|---|---|---|
| Grp | Formulation | Dose (μg) | Days | Days | Virus | Mice |
| 1 | H1-ss-np | 1 | 7 and 21 | 0, 21, | A/CA/04/2009 | 10 |
| 2 | H1-ss-np + | 1 | | 35, | (H1N1), | 10 |
| | 20 μg Alhydrogel | | | and 63 | day 49 | |
| 3 | H1-ss-np (U)+ | 1 | 0 and 21 | | | 10 |
| | Alhydrogel + | | | | | |
| | CPG 7909 (B) | | | | | |
| | Formulated 1:20:25 | | | | | |
| 4 | H1-ss-np (U)+ | 1 | | | | 10 |
| | Alhydrogel + | | | | | |
| | CPG 7909 (U) | | | | | |
| | Formulated 1:20:25 | | | | | |
| 5 | H1-ss-np (B) + | 1 | | | | 10 |
| | Alhydrogel + | | | | | |
| | CPG 7909 (B) | | | | | |
| | Formulated 1:20:25 | | | | | |
| 6 | H1-ss-np (U) + | 1 | | | | 10 |
| | Alhydrogel + | | | | | |
| | CPG 7909 (B) | | | | | |
| | Formulated 2:20:13 | | | | | |
| 7 | H1-ss-np (U) + | 1 | | | | 10 |
| | Alhydrogel + | | | | | |
| | CPG 7909 (U) | | | | | |
| | Formulated 2:20:13 | | | | | |
| 8 | H1-ss-np (B)+ | 1 | | | | 10 |
| | Alhydrogel + | | | | | |
| | CPG 7909 (B) | | | | | |
| | Formulated 2:20:13 | | | | | |
| 9 | Vehicle | 1 | | | | 10 |

Ratios in Table 8 refer to the amount (in μg) of H1-ss-np:Alhydrogel:CpG that each mouse received.

### Results

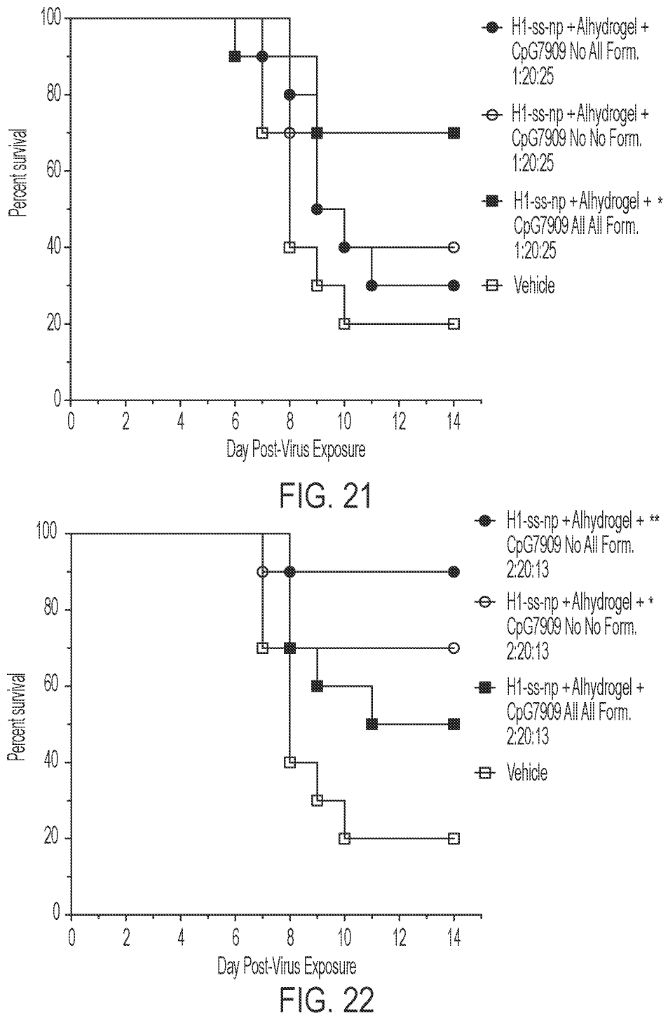

FIG. 21 shows survival curves following vaccination with formulations containing a 1:20:25 ratio of H1-ss-np:Alhydrogel:CPG 7909, and after challenge with influenza A/CA/04/2009 (H1N1pdm) virus. The formulation, H1-ss-np (B)+Alhydrogel+CPG 7909 (B) (Formulated 1:20:25 [all antigen and CPG 7909 bound to Alhydrogel]), was the only vaccine to provide statistically significant protection where seven of ten (70%) mice survived the infection (Panel A). The formulation, H1-ss-np (U)+Alhydrogel+CPG 7909 (B) (Formulated 1:20:25 [no antigen and all CPG 7909 bound to Alhydrogel]), only protected three of ten (30%) mice from mortality. The formulation, H1-ss-np (U)+Alhydrogel+CPG 7909 (U) (Formulated 1:20:25 [no antigen or CPG 7909 bound to Alhydrogel]), was able to protect four of ten (40%) of mice from mortality.

FIG. 22 shows survival curves following vaccination with formulations containing a 2:20:13 ratio of H1-ss-np:Alhydrogel:CPG 7909, followed by challenge with influenza A/CA/04/2009 (H1N1pdm) virus. Mice vaccinated with the formulation H1-ss-np (U)+Alhydrogel+CPG 7909 (B) (Formulated 2:20:13 [no antigen and all CPG 7909 bound to Alhydrogel]) and mice vaccinated with the formulation H1-ss-np (U)+Alhydrogel+CPG 7909 (B) (Formulated 2:20:13 [no antigen or CPG 7909 bound to Alhydrogel]) were significantly protected, statistically, from infection where nine of ten (90%) and seven of ten (70%), respectively, of mice survived the infection (Panel A). Five of ten (50%) mice vaccinated with the formulation H1-ss-np (B)+Alhydrogel+CPG 7909 (B) (Formulated 2:20:13[all antigen and CPG 7909 bound to Alhydrogel]) survived the infection.

Table 9 summarizes the percent survival results shown in FIGS. 21 and 22.

TABLE 9
Survival Rates

| | | No. of Survivors/ | Percent | |
|---|---|---|---|---|
| Grp | Adjuvant | Total Number of Mice | Survival | P Value* |
| 1 | H1-ss-np | 3/10 | 30% | NS |
| 2 | H1-ss-np + 20 μg Alhydrogel | 1/10 | 10% | NS |
| 3 | H1-ss-np (U) + Alhydrogel + CPG | 3/10 | 30% | NS |
| | 7909 (B) Formulated 1:20:25 | | | |
| 4 | H1-ss-np (U) + Alhydrogel + CPG | 4/10 | 40% | NS |
| | 7909 (U) Formulated 1:20:25 | | | |
| 5 | H1-ss-np (B) + Alhydrogel + CPG | 7/10 | 70% | <0.05 |
| | 7909 (B) Formulated 1:20:25 | | | |
| 6 | H1-ss-np (U) + Alhydrogel + CPG | 9/10 | 90% | <0.01 |
| | 7909 (B) Formulated 2:20:13 | | | |
| 7 | H1-ss-np (U) + Alhydrogel + CPG | 7/10 | 70% | <0.05 |
| | 7909 (U) Formulated 2:20:13 | | | |
| 8 | H1-ss-np (B) + Alhydrogel + CPG | 5/10 | 50% | NS |
| | 7909 (B) Formulated 2:20:13 | | | |
| 9 | Vehicle | 2/10 | 20% | — |

*Compared to placebo-treated (PBS) mice
NS—Not statistically significant

Additional formulations of CpG ODN, Alhydrogel, and phosphate were evaluated in an in vivo challenge study. A tabulated description of the experimental design is in Table 10. Two hundred female BALB/c mice were used for this study. Briefly, 15 groups of mice (n=10) were vaccinated by the intramuscular (IM) route with H1 and H10 antigens and one of 15 adjuvant formulations at Day 0, Day 21, and Day 42 (Groups 1-15). Five additional groups (Groups 16-20) acted as controls. Two groups of mice (Groups 16 and 17; n=10) were vaccinated with H1 antigen in one of two adjuvant formulations at Day 0, Day 21, and Day 42 to act as monovalent controls. A positive control group (Group 18; n=10) was vaccinated with inactivated influenza virus homologous to the strain used for subsequent challenge. Finally, two negative control groups (n=10) were used, a negative adjuvant control was vaccinated with adjuvant only (Group 19; using the same adjuvant formulation as used in Group 1), and a negative vehicle control was vaccinated with vehicle only (Group 20). At Day 70 animals were challenged by the intranasal (IN) route with 3× LD50 of influenza H1N1 A/CA/04/2009. Post-challenge animals were monitored twice daily for mortality to Day 84 (14 days post-challenge). Survival rates are reported in Table 11.

TABLE 10
Study design for adjuvant formulation evaluation in H1N1 infected mice

| | | H1 Antigen | H10 Antigen | CpG | Alhydrogel | Phosphate |
|---|---|---|---|---|---|---|
| Group | N | (μg) | (μg) | (μg) | (μg) | (mM) |
| 1 | 10 | 5 | 5 | 100 | 60 | 0.4 |
| 2 | 10 | 5 | 5 | 50 | 60 | 0.4 |
| 3 | 10 | 5 | 5 | 50 | 30 | 0.4 |
| 4 | 10 | 5 | 5 | 25 | 30 | 0.4 |
| 5 | 10 | 5 | 5 | 100 | 60 | 5 |
| 6 | 10 | 5 | 5 | 50 | 60 | 5 |
| 7 | 10 | 5 | 5 | 30 | 60 | 5 |
| 8 | 10 | 5 | 5 | 50 | 30 | 5 |
| 9 | 10 | 5 | 5 | 25 | 30 | 5 |
| 10 | 10 | 5 | 5 | 15 | 30 | 5 |
| 11 | 10 | 5 | 5 | 100 | 60 | 20 |
| 12 | 10 | 5 | 5 | 50 | 60 | 20 |
| 13 | 10 | 5 | 5 | 50 | 30 | 20 |
| 14 | 10 | 5 | 5 | 25 | 30 | 20 |
| 15 | 10 | 5 | 5 | 0 | 0 | 0 |
| 16 | 10 | 5 | 0 | 100 | 60 | 0.4 |
| 17 | 10 | 5 | 0 | 50 | 30 | 0.4 |
| 18 | 10 | 5 μg inactivated virus influenza H1N1 A/CA/04/2009 vaccine | | | | |
| 19 | 10 | 0 | 0 | 100 | 60 | 0.4 |

| | | | | | |
|---|---|---|---|---|---|
| 20 | 10 | Formulation Buffer | 0 | 0 | 0.4 |

Vaccination Schedule: IM at Days 0, 21 & 42
Virus Challenge: Day 70 with 3x LD50 influenza H1N1 A/CA/04/2009 delivered IN
Measurements:
Survival Day 84
Serum for possible future analysis (Days 0, 35, 56 and 84)

TABLE 11
Survival of Mice Lethally Infected with H1N1 A/CA/04/2009

| | Vaccine composition | | | | |
|---|---|---|---|---|---|
| | H1 + H10 Antigen | CpG | Alhydrogel | Phosphate | |
| Group | (μg) | (μg) | (μg) | (mM) | % Survival |
| 1 | 5 + 5 | 100 | 60 | 0.4 | 90 |
| 2 | 5 + 5 | 50 | 60 | 0.4 | 70 |
| 3 | 5 + 5 | 50 | 30 | 0.4 | 100 |
| 4 | 5 + 5 | 25 | 30 | 0.4 | 80 |
| 5 | 5 + 5 | 100 | 60 | 5 | 80 |
| 6 | 5 + 5 | 50 | 60 | 5 | 90 |
| 7 | 5 + 5 | 30 | 60 | 5 | 50 |
| 8 | 5 + 5 | 50 | 30 | 5 | 90 |
| 9 | 5 + 5 | 25 | 30 | 5 | 80 |
| 10 | 5 + 5 | 15 | 30 | 5 | 80 |
| 11 | 5 + 5 | 100 | 60 | 20 | 90 |
| 12 | 5 + 5 | 50 | 60 | 20 | 90 |
| 13 | 5 + 5 | 50 | 30 | 20 | 80 |
| 14 | 5 + 5 | 25 | 30 | 20 | 90 |
| 15 | 5 + 5 | 0 | 0 | 0 | 100 |
| 16 | 5 + 0 | 100 | 60 | 0.4 | 90 |
| 17 | 5 + 0 | 50 | 30 | 0.4 | 90 |
| 18 | 5 μg Inactivated virus influenza H1N1 A/CA/04/2009 vaccine | | | | 100 |
| 19 | 0 + 0 | 100 | 60 | 0.4 | 20 |
| 20 | 0 + 0 | 0 | 0 | 0.4 | 0 |

Formulations of CpG ODN, Alhydrogel, and phosphate were also evaluated in another in vivo heterologous challenge study with influenza H3N2 A/Victoria/3/75. A tabulated description of the experimental design is in Table 12. Two hundred and fifty female BALB/c mice were used for this study. Briefly, 15 groups of mice (n=10) were vaccinated by the intramuscular (IM) route with H1 and H10 antigens and one of 15 adjuvant formulations at Day 0, Day 21, and Day 42 (Groups 1-15). Ten additional groups (Groups 16-25) acted as controls. Two groups of mice (Groups 16 and 17; n=10) were vaccinated with H1 antigen in one of two adjuvant formulations at Day 0, Day 21, and Day 42 to act as negative monovalent controls. Two additional groups of mice (Groups 18 and 19; n=10) were vaccinated with H10 antigen in one of two adjuvant formulations at Day 0, Day 21, and Day 42 to act as monovalent controls. Two positive control groups (Group 20 and 21; n=10) were vaccinated with inactivated influenza virus homologous to the strain used for subsequent challenge. Finally, four negative control groups (n=10) were used: two negative adjuvant control groups were vaccinated with CpG, Alhydrogel, and phosphate buffer only (Groups 22 and 23; using the same formulation as used in Group 1), and two negative vehicle control were vaccinated with vehicle only, no antigen or adjuvants, (Groups 24 and 25). At Day 70, animals were challenged by the intranasal (IN) route with 3×LD50 of influenza H3N2 A/Victoria/3/75. Post-challenge animals were monitored twice daily for mortality to Day 84 (14 days post-challenge). Survival rates are reported in Table 13.

TABLE 12
Study design for adjuvant formulation evaluation in H3N2 infected mice

| | | H1 Antigen | H10 Antigen | CpG | Alhydrogel | Phosphate |
|---|---|---|---|---|---|---|
| Group | N | (μg) | (μg) | (μg) | (μg) | (mM) |
| 1 | 10 | 5 | 5 | 100 | 60 | 0.4 |
| 2 | 10 | 5 | 5 | 50 | 60 | 0.4 |
| 3 | 10 | 5 | 5 | 50 | 30 | 0.4 |
| 4 | 10 | 5 | 5 | 25 | 30 | 0.4 |
| 5 | 10 | 5 | 5 | 100 | 60 | 5 |
| 6 | 10 | 5 | 5 | 50 | 60 | 5 |
| 7 | 10 | 5 | 5 | 30 | 60 | 5 |
| 8 | 10 | 5 | 5 | 50 | 30 | 5 |
| 9 | 10 | 5 | 5 | 25 | 30 | 5 |
| 10 | 10 | 5 | 5 | 15 | 30 | 5 |
| 11 | 10 | 5 | 5 | 100 | 60 | 20 |
| 12 | 10 | 5 | 5 | 50 | 60 | 20 |
| 13 | 10 | 5 | 5 | 50 | 30 | 20 |
| 14 | 10 | 5 | 5 | 25 | 30 | 20 |
| 15 | 10 | 5 | 5 | 0 | 0 | 0 |
| 16 | 10 | 5 | 0 | 100 | 60 | 0.4 |
| 17 | 10 | 5 | 0 | 50 | 30 | 0.4 |
| 18 | 10 | 0 | 5 | 100 | 60 | 0.4 |
| 19 | 10 | 0 | 5 | 50 | 30 | 0.4 |
| 20 | 10 | 5 μg inactivated virus influenza H3N2 A/Victoria/3/75 vaccine | | | | |
| 21 | 10 | 5 μg inactivated virus influenza H3N2 A/Victoria/3/75 vaccine | | | | |
| 22 | 10 | 0 | 0 | 100 | 60 | 0.4 |
| 23 | 10 | 0 | 0 | 100 | 60 | 0.4 |

| | | | | | |
|---|---|---|---|---|---|
| 24 | 10 | Formulation Buffer | 0 | 0 | 0.4 |
| 25 | 10 | Formulation Buffer | 0 | 0 | 0.4 |

Vaccination Schedule: IM at Days 0, 21 & 42
Virus Challenge: Day 70 - 3x LD50 influenza H3N2 A/Victoria/3/75 delivered IN
Measurements:
Survival Day 84
Serum for possible future analysis (Days 0, 35, 56 and 84)

TABLE 13
Survival of Mice Lethally Infected with H3N2 A/Victoria/3/75

| | Vaccine composition | | | | |
|---|---|---|---|---|---|
| | H1 + H10 Antigen | CpG | Alhydrogel | Phosphate | |
| Group | (μg) | (μg) | (μg) | (mM) | % Survival |
| 1 | 5 + 5 | 100 | 60 | 0.4 | 0 |
| 2 | 5 + 5 | 50 | 60 | 0.4 | 30 |
| 3 | 5 + 5 | 50 | 30 | 0.4 | 20 |
| 4 | 5 + 5 | 25 | 30 | 0.4 | 30 |
| 5 | 5 + 5 | 100 | 60 | 5 | 10 |
| 6 | 5 + 5 | 50 | 60 | 5 | 80 |
| 7 | 5 + 5 | 30 | 60 | 5 | 40 |
| 8 | 5 + 5 | 50 | 30 | 5 | 50 |
| 9 | 5 + 5 | 25 | 30 | 5 | 30 |
| 10 | 5 + 5 | 15 | 30 | 5 | 30 |
| 11 | 5 + 5 | 100 | 60 | 20 | 50 |
| 12 | 5 + 5 | 50 | 60 | 20 | 40 |
| 13 | 5 + 5 | 50 | 30 | 20 | 40 |
| 14 | 5 + 5 | 25 | 30 | 20 | 50 |
| 15 | 5 + 5 | 0 | 0 | 0 | 60 |
| 16 | 0 + 5 | 100 | 60 | 0.4 | 40 |
| 17 | 0 + 5 | 50 | 30 | 0.4 | 20 |
| 18 | 5 + 0 | 100 | 60 | 0.4 | 20 |
| 19 | 5 + 0 | 50 | 30 | 0.4 | 30 |
| 20 | 5 μg Inactivated virus influenza H3N2 A/Victoria/3/75 vaccine | | | | 100 |
| 21 | 5 μg Inactivated virus influenza H3N2 A/Victoria/3/75 vaccine | | | | 100 |
| 22 | 0 + 0 | 100 | 60 | 0.4 | 30 |
| 23 | 0 + 0 | 100 | 60 | 0.4 | 10 |
| 24 | 0 + 0 | 0 | 0 | 0.4 | 10 |
| 25 | 0 + 0 | 0 | 0 | 0.4 | 0 |

The percent survival result from Group 15 of Table 13 is believed to be an outlier based on historical studies and data. Further experiments are being conducted to verify and better understand this result, which include generating additional data for statistical analysis.

### Example 11. Assessment of Antigen and Adjuvant Binding

The binding isotherms of CpG ODN to Alhydrogel (0.0600 w/v of aluminum) at different concentrations of phosphate were evaluated in formulation buffers with different phosphate concentrations. The percentage of bound CpG ODN was measured after mixing CpG ODN in phosphate buffer with Alhydrogel together and allowing it to equilibrate. The results are summarized in Table 14. When the percentage bound CpG ODN drops to 95% or less, it is believed CpG ODN has saturated the Alhydrogel binding sites and further addition of the type A HA-ss-np antigen(s) in the mixture will not substantially bind to Alhydrogel.

Selected formulations where CpG saturates the Alhydrogel binding sites based on the isotherm study were further mixed with H1-ss-np and H10-ss-np antigens, 120 μg/mL of each antigen, and the percentage of antigens adsorbed on Alhydrogel and antigen relative potency were measured. The results are summarized in Tables 15 and 16. Table 15 shows the percent adsorption of type A HA-ss-np antigens on Alhydrogel in selected adjuvant formulations where the CpG ODN saturates binding to Alhydrogel. Table 16 shows the relative potency, as measured by Biolayer Interferometry (BLI), of Type A HA-ss-np antigens in selected adjuvant formulations where the CpG ODN saturates binding to Alhydrogel. H1-ss-np and H10-ss-np were measured separately; the first number in the table is for H1-ss-np, and the second number in the table is for H10-ss-np. In all tested formulations, non-substantial binding between antigens and Alhydrogel was demonstrated, and these formulations retained their relative potency.

TABLE 14
Binding Isotherms of CpG ODN to Alhydrogel

| Phosphate | CPG concentration (w/v %) | | | | | | | | | |
|---|---|---|---|---|---|---|---|---|---|---|
| concentration (mM) | 0.005 | 0.010 | 0.015 | 0.020 | 0.025 | 0.030 | 0.035 | 0.050 | 0.075 | 0.100 |
| 0 | 99% | 100% | 100% | 100% | 100% | 100% | 100% | 100% | 73% | 57% |
| 0.5 | 99% | 100% | 99% | 100% | 100% | 100% | 100% | 76% | 54% | 41% |
| 1 | 99% | 99% | 99% | 100% | 99% | 99% | 94% | 64% | 47% | 41% |
| 5 | 98% | 100% | 99% | 100% | 95% | 85% | 73% | 52% | 37% | 32% |
| 10 | 98% | 100% | 99% | 97% | 86% | 73% | 47% | 47% | 30% | 22% |
| 20 | 98% | 100% | 97% | 84% | 74% | 61% | 58% | 38% | 32% | 18% |
| 50 | 98% | 91% | 71% | 58% | 51% | 40% | 46% | 22% | 17% | 9% |
| 100 | 84% | 58% | 43% | 35% | 43% | 21% | 21% | 10% | 10% | 5% |

TABLE 15
Percent Adsorption of HA-ss-np Antigens on Alhydrogel

| Phosphate concentration | CpG concentration (w/v %) | | | |
|---|---|---|---|---|
| (mM) | 0.025% | 0.030% | 0.050% | 0.100% |
| 0.5 | NT | NT | NT | ≤5% |
| 5 | ≤5% | ≤5% | NT | ≤5% |
| 10 | NT | NT | ≤5% | ≤5% |
| 20 | NT | NT | ≤5% | ≤5% |

*NT: Not Tested

TABLE 16
Relative Potency of HA-ss-np Antigens

| Phosphate | CpG concentration (w/v %) | | |
|---|---|---|---|
| concentration (mM) | 0.025% | 0.050% | 0.100% |
| 0.5 | NT | NT | ≥85%, ≥85% |
| 5 | ≥85%, ≥85% | NT | NT |
| 10 | NT | ≥85%, ≥85% | ≥85%, ≥85% |
| 20 | NT | NT | ≥85%, ≥85% |

*NT: Not Tested
First number: H1-ss-np;
second number: H10-ss-np

What is claimed is:

1. A vaccine composition comprising an influenza Type A hemagglutinin stabilized stem nanoparticle (HA-ss-np); an aluminum hydroxide; a synthetic oligodeoxynucleotide containing at least one CpG motif (CpG ODN); and a phosphate salt,
wherein the HA-ss-np is not substantially adsorbed to the aluminum hydroxide, and wherein at least a portion of the CpG ODN is adsorbed to the aluminum hydroxide in the composition.

2. The vaccine composition of claim 1, wherein weight/weight (w/w) ratio of aluminum in the aluminum hydroxide to the CpG ODN is about 1:1 to about 1:5.

3. The vaccine composition of claim 2, wherein the w/w ratio of the aluminum in the aluminum hydroxide to the CpG ODN is about 1:1 to about 1:2.

4. The vaccine composition of any one of claims 1 to 3, wherein the aluminum hydroxide is about 0.02% weight/volume (w/v) to about 0.10% w/v of the composition.

5. The vaccine composition of claim 4, wherein the aluminum hydroxide is about 0.04% w/v to about 0.08% w/v of the composition.

6. The vaccine composition of any one of claims 1 to 5, wherein the CpG ODN is CPG 7909 or CPG 1826.

7. The vaccine composition of any one of claims 1 to 6, wherein the CpG ODN is about 0.01% w/v to about 0.1% w/v of the composition.

8. The vaccine composition of any one of claims 1 to 6, wherein the CpG ODN is about 0.05% w/v to about 0.2% w/v of the composition.

9. The vaccine composition of claim 8, wherein the CpG ODN is about 0.08% w/v to about 0.15% w/v of the composition.

10. The vaccine composition of any one of claims 1 to 9, wherein the aluminum hydroxide is about 0.06% w/v of the composition and the CpG ODN is about 0.10% w/v of the composition.

11. The vaccine composition of any one of claims 1 to 9, wherein the aluminum hydroxide is about 0.06% w/v of the composition and the CpG ODN is about 0.05% w/v of the composition.

12. The vaccine composition of any one of claims 1 to 11, wherein the phosphate salt is sodium phosphate monobasic monohydrate.

13. The vaccine composition of any one of claims 1 to 12, wherein the phosphate salt is at a concentration of about 0.3 mM to about 0.7 mM in the composition.

14. The vaccine composition of claim 13, wherein the phosphate salt is at a concentration of about 0.3 mM to about 0.4 mM in the composition.

15. The vaccine composition of any one of claims 1 to 12, wherein the phosphate salt is at a concentration of about 1 mM to about 20 mM in the composition.

16. The vaccine composition of claim 15, wherein the phosphate salt is at a concentration of about 2 mM to about 10 mM in the composition.

17. The vaccine composition of any one of claims 1 to 16, wherein about 10% to about 100% of the CpG ODN in the composition is adsorbed to the aluminum hydroxide.

18. The vaccine composition of any one of claims 1 to 17, wherein at least 90% of adsorption sites in the aluminum hydroxide is bound to the CpG ODN.

19. The vaccine composition of any one of claims 1 to 18, wherein pH of the composition is about 6.5 to about 8.

20. The vaccine composition of claim 19, wherein pH of the composition is about 7.2 to about 7.8.

21. The vaccine composition of any one of claims 1 to 20, further comprising QS-21.

22. The vaccine composition of any one of claims 1 to 21, further comprising a surfactant, a salt, a buffering agent, a cryoprotectant, or a combination thereof.

23. The vaccine composition of any one of claims 1 to 22, wherein the HA-ss-np is about 0.005% w/v to about 0.05% w/v of the composition.

24. The vaccine composition of claim 23, wherein the HA-ss-np is about 0.008% w/v to about 0.03% w/v of the composition.

25. The vaccine composition of any one of claims 1 to 24, wherein the HA-ss-np comprises a hemagglutinin stabilized stem (HA-ss) from a Group 1 influenza A virus, an HA-ss from a Group 2 influenza A virus, or a combination thereof.

26. A vaccine composition comprising:
about 0.002% w/v to about 0.015% w/v of a first hemagglutinin stabilized stem nanoparticle (HA-ss-np) comprising an HA-ss from a Group 1 influenza A virus;
about 0.002% w/v to about 0.015% w/v of a second hemagglutinin stabilized stem nanoparticle (HA-ss-np) comprising an HA-ss from a Group 2 influenza A virus;
about 0.02% w/v to about 0.08% w/v of an aluminum hydroxide;
about 0.08% w/v to about 0.12% w/v of CPG 7909;
about 0.3 mM to about 0.7 mM sodium phosphate monobasic monohydrate;
about 0.01% w/v to about 0.02% w/v of a polysorbate;
about 1 mM to about 10 mM Tris-HCl;
about 50 mM to about 200 mM NaCl; and
about 1% w/v to about 10% w/v sucrose,
wherein the first HA-ss-np and the second HA-ss-np are not substantially adsorbed to the aluminum hydroxide, and wherein at least a portion of the CPG 7909 is adsorbed to the aluminum hydroxide.

27. A vaccine composition comprising:
about 0.002% w/v to about 0.015% w/v of a first hemagglutinin stabilized stem nanoparticle (HA-ss-np) comprising an HA-ss from a Group 1 influenza A virus;
about 0.002% w/v to about 0.015% w/v of a second hemagglutinin stabilized stem nanoparticle (HA-ss-np) comprising an HA-ss from a Group 2 influenza A virus;
about 0.02% w/v to about 0.08% w/v of an aluminum hydroxide;
about 0.010% w/v to about 0.10% w/v of CPG 7909;
about 1 mM to about 10 mM sodium phosphate monobasic monohydrate;
about 0.01% w/v to about 0.02% w/v of a polysorbate;
about 1 mM to about 10 mM Tris-HCl;
about 50 mM to about 200 mM NaCl; and
about 1% w/v to about 10% w/v sucrose,
wherein the first HA-ss-np and the second HA-ss-np are not substantially adsorbed to the aluminum hydroxide, and wherein at least a portion of the CPG 7909 is adsorbed to the aluminum hydroxide.

28. A method of inducing an immunological response against an influenza virus in a subject in need thereof, comprising administering an immunologically effective amount of the vaccine composition of any one of claims 1 to 27 to the subject.

29. A method of inducing an immunological response against an influenza virus in a subject in need thereof, comprising administering a dose of about 20 μg to about 300 μg of an HA-ss-np in a vaccine composition, wherein the vaccine composition further comprises an aluminum hydroxide; CpG ODN; and a phosphate salt, and wherein the HA-ss-np is not substantially adsorbed to the aluminum hydroxide, and wherein at least a portion of the CpG ODN is adsorbed to the aluminum hydroxide.

30. The method of claim 28 or 29, comprising administering: about 10 μg to about 150 μg of a first HA-ss-np comprising an HA from a Group 1 influenza virus; and/or about 10 μg to about 150 μg of a second HA-ss-np comprising an HA from a Group 2 influenza virus.

31. The method of claim 30, comprising administering about 30 μg of the first HA-ss-np and/or about 30 μg of the second HA-ss-np.

32. The method of claim 30, comprising administering about 120 μg of the first HA-ss-np and/or about 120 μg of the second HA-ss-np.

33. The method of any one of claims 28 to 32, comprising administering a single dose of the vaccine composition.

34. The method of any one of claims 28 to 32, comprising administering two or more doses of the vaccine composition, wherein a second dose is administered about 2 weeks to about 4 weeks after a first dose.

35. A method of producing a vaccine composition, comprising:
combining HA-ss-np with an adjuvant mixture, wherein the adjuvant mixture comprises a diluent solution comprising a phosphate salt; aluminum hydroxide; and CpG ODN, wherein the adjuvant mixture comprises CpG ODN-adsorbed aluminum hydroxide, and wherein the HA-ss-np is not substantially adsorbed to the aluminum hydroxide.

36. The method of claim 35, wherein the adjuvant mixture is prepared at least 10 minutes prior to the combining step.


SEQUENCE LISTING (16 sequences)

SEQ ID NO: 1 - protein, 383 aa, synthetic construct
MKAKLLVLLCTFTATYADTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 2 - protein, 381 aa, synthetic construct
MKAKLLVLLCTFTATYADTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 3 - protein, 388 aa, synthetic construct
MYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 4 - protein, 386 aa, synthetic construct
MYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 5 - protein, 366 aa, synthetic construct
DTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 6 - protein, 364 aa, synthetic construct
DTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 7 - protein, 372 aa, synthetic construct
LDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 8 - protein, 370 aa, synthetic construct
LDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNINSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 9 - protein, 215 aa, synthetic construct
MKAKLLVLLCTFTATYADTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKID

SEQ ID NO: 10 - protein, 220 aa, synthetic construct
MYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNIN

SEQ ID NO: 11 - protein, 198 aa, synthetic construct
DTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKID

SEQ ID NO: 12 - protein, 204 aa, synthetic construct
LDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVELMEQGGPDCYLAELLVAMLNQHVIDMADSEMRNLYERVRKQLRQNAEEDGKGCFEIYHACDDSCMESIRNNTYDHSQYREEALLNRLNIN

SEQ ID NO: 13 - protein, 165 aa, synthetic construct
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 14 - protein, 163 aa, synthetic construct
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 15 - skipped

SEQ ID NO: 16 - DNA, 24 bp, synthetic construct
  source 1..24: mol_type = other DNA
tcgtcgttttgtcgttttgtcgtt